(19) United States
(12) Patent Application Publication
(10) Pub. No.: US 2010/0129790 A1
(43) Pub. Date: May 27, 2010

(54) Method for Determining Phospholipidosis

(75) Inventors: Hiroshi Sawada, Osaka (JP); Ikuo Mori, Osaka (JP); Kenji Takami, Osaka (JP)

Correspondence Address: EDWARDS ANGELL PALMER & DODGE LLP, P.O. BOX 55874, BOSTON, MA, 02205, US

(73) Assignee: TAKEDA PHARMACEUTICAL LIMITED, CHUO-KU OSAKA-SHI OSAKA (JP)

(21) Appl. No.: 11/884,546

(22) PCT Filed: Feb. 16, 2006
(86) PCT No.: PCT/JP06/03205

§ 371 Date: Aug. 17, 2007

(30) Foreign Application Priority Data
Feb. 17, 2005 (JP) 0406982005
Apr. 26, 2005 (JP) 128412/2005

(51) Int. Cl. C12Q 1/68

(52) U.S. Cl. 435/6

(57) ABSTRACT

It is intended to provide a rapid, simple and noninvasive novel in vivo method for determining phospholipidosis (PLsis) by detecting a PLsis marker gene. More specifically, it is intended to provide a method for determining phospholipidosis in a mammal comprising the step of detecting change in expression of one or more genes whose expression changes correlated with the onset of phospholipidosis in a sample collected from the mammal, in which at least one of the genes comprises a base sequence identical or substantially identical to the base sequence represented by any of SEQ ID NO: n (wherein n is an odd number between 1 and 57, an integer number between 59 and 63, an even number between 64 and 98, 100 or 101.)

### TECHNICAL FIELD

The present invention relates to a method for determining phospholipidosis and tools therefor. More particularly, the present invention relates to a method for determining phospholipidosis (e.g., drug-induced phospholipidosis etc.) with expression variations of blood marker genes as indices, and reagents, kits and the like for the detection of the blood marker genes.

### BACKGROUND ART

Phospholipidosis (hereinafter sometimes to be abbreviated as “PLsis”) is defined to be a phenomenon where phospholipid accumulates in excess in the cell. It is caused by abnormal homeostasis of lipid metabolism including that induced hereditarily, as well as many pharmaceutical agents such as antidepressants, antianginal drugs, antimalarial drugs, anti-anorectic drugs, hypolipidemic drugs and the like or metabolites thereof. In PLsis, phospholipid is mainly accumulated in lysosome and a cyclic or elliptic myelin-like structure (lamellar body) is observed electron microscopically. While the expression mechanism of toxicity has not been completely elucidated, it is considered to be caused by 1) inhibition of lysosomal enzyme (mainly phospholipid degradation enzyme (phospholipase)) activity by compound, 2) inhibition of transport pathway involved in phospholipid metabolism by compound, 3) inhibition of degradation of complex by the formation of a complex of compound and phospholipid, 4) promotion of synthesis of phospholipid by compound and the like.

Many of the PLsis-inducing compounds have a structure comprising, in a molecule, a hydrophobic domain and a positively charged hydrophilic domain in combination (cationic amphiphilic drug; CAD). In recent years, along with the progress of genome analysis, the value of orphan receptors as drug discovery targets has been recognized and the development of agonists or antagonists against the receptors is being undertaken. However, because such compounds act on receptors, they mostly have CAD structures and an increasing number of incidents have been experienced where expression of PLsis prevents development of pharmaceutical products. Moreover, the pharmaceutical products that have already been approved include those reported to induce PLsis as a side effect. Therefore, the development of an efficient evaluation or prediction system for PLsis-inducibility of a drug has been urgently desired.

Incidentally, microarray technology for simultaneously monitoring expression of several thousand to several tens of thousand kinds of mRNAs (comprehensive gene expression analysis, transcriptomics) has been actively used in various kinds of fields of medicine and biology. In the field of toxicology, too, this technology has been utilized for the elucidation of the mechanism of toxicity expression and the study of toxicity prediction, and is expected to be a new study field called toxicogenomics. Toxicity phenomenon is considered to accompany not only independent changes in one or several genes, but also integral variations where many genes are correlated, such as interaction of genes, cascade and the like. Based thereon, it is expected that the behavior of molecules involved in toxicity expression can be comprehensively perceived by the use of a technique of microarray capable of analysis at transcriptome level.

The present inventors previously analyzed comprehensively gene expression in human cultured cells exposed to a known PLsis-inducing compound using microarray, identified and reported marker genes that showed remarkably varying expression on inducing PLsis (Sawada, H. et al., Toxicol. Sci., 83: 282-92 (2005)). Since an in vitro screening system using cultured cells can evaluate many samples in a short time using a small amount of a compound, it is a superior tool for rapidly predicting the presence or absence of PLsis-inducibility and efficiently optimizing a structure in the initial step of drug discovery when synthesis of the compound is restricted.

On the other hand, the results in an in vitro evaluation system do not always reflect the in vivo toxicity expression. For example, a compound that induces PLsis in cells by in vitro screening can be said to have a PLsis-inducing potential. However, the compound may not show toxicity when it is rapidly metabolized in vivo and the like, and vice versa. Alternatively, when the concentration at which the efficacy is exhibited is sufficiently lower than the concentration at which the toxicity is expressed, staging up to the preclinical or clinical stage is possible.

Therefore, construction of an in vivo evaluation system capable of rapidly and conveniently determining the presence or absence of the expression of drug-induced PLsis due to the administration of a compound in the later stage of development or after placing on the market has been demanded. In such an evaluation system, since experiment animals or volunteers for clinical trial or patients are the subjects, collection of a sample for an analysis is desirably as noninvasive as possible. On the other hand, since utilization of transcriptomics requires a cell-containing sample, the sample for an analysis is preferably blood, lymphocyte and the like.

When a biological sample such as blood, lymph fluid and the like is used as an analysis target, a novel and more suitable marker gene (genes), which is different from marker genes used for in vitro evaluation systems, may be found (e.g., see Rockett, J. C. et al., Toxicol. Sci., 69: 49-59 (2002)). However, there is no report relating to a particular marker gene capable of highly accurately determining the PLsis-inducibility in an in vivo evaluation system.

### DISCLOSURE OF THE INVENTION

It is an object of the present invention to provide an in vivo evaluation system of PLsis, which comprises identifying a gene showing varying expression in blood and the like in correlation with PLsis expression, or a PLsis marker gene, and using expression variation of the gene as an index.

In an attempt to solve the aforementioned problems, the present inventors have performed comprehensive analysis of gene expression using a microarray with, as a sample, the blood from rats administrated with various known PLsis-inducing compounds for 3 days or 7 days. As a result, the present inventors have succeeded in identifying genes that showed remarkably varying expression for all of these compounds for each administration period. In addition, for many compounds, most of these genes showed expression variation by administration for 3 days and administration for 7 days. The present inventors further studied based on these findings and completed the present invention.

Accordingly, the present invention provides

[1] a reagent for determining phospholipidosis in a mammal, which comprises a nucleic acid capable of hybridizing to a nucleic acid having a base sequence shown by any of SEQ ID NO n (wherein n is an odd number between 1 and 57, an integer number between 59 and 63, an even number between 64 and 98, 100 or 101) under high stringent conditions and/or a nucleic acid capable of hybridizing to a nucleic acid having a base sequence complementary to the base sequence under high stringent conditions;
[2] a kit for determining phospholipidosis in a mammal, which comprises two or more reagents containing a nucleic acid capable of hybridizing to a transcription product of a gene showing varying expression in correlation with expression of phospholipidosis under high stringent conditions and/or a nucleic acid capable of hybridizing to a nucleic acid having a base sequence complementary to the transcription product under high stringent conditions, wherein,
(a) at least one reagent is the reagent of the above-mentioned [1], and
(b) when two or more reagents of the above-mentioned [1] are contained, each reagent can detect expression of different genes;
[3] a method for determining phospholipidosis in a mammal, which comprises detecting expression variation of one or more genes showing expression variation in correlation with phospholipidosis expression, in a sample from a mammal, wherein at least one gene has the same or substantially the same base sequence as the base sequence shown by any of SEQ ID NO n (wherein n is an odd number between 1 and 57, an integer number between 59 and 63, an even number between 64 and 98, 100 or 101);
[4] the method of the above-mentioned [3], wherein the mammal is administered with a compound or exposed to the compound, and phospholipidosis is caused by the compound;
[5] the method of the above-mentioned [3], wherein the mammal is human;
[6] the method of the above-mentioned [3], wherein the mammal is rat, mouse, dog or monkey; and
[7] the method of the above-mentioned [3], wherein the sample is blood or lymphocyte;
and the like.

The method for determining PLsis of the present invention is characterized by detection of expression variation of a PLsis marker gene in a sample from a mammal, and affords a superior effect in that the expression of PLsis can be determined noninvasively and with good precision, as compared to conventional in vivo toxicity tests and the like.

### BEST MODE FOR EMBODYING THE INVENTION

The present invention provides a reagent for determining PLsis containing a nucleic acid capable of detecting the expression of a gene showing varying expression in correlation with PLsis expression (i.e., PLsis marker gene). As used herein, by “varying expression in correlation with PLsis expression” is meant a statistically significant tendency toward substantial increase or decrease in the expression, when one or more mammalian tissues show histopathological finding of PLsis. By the “substantial increase or decrease” is meant an increase to not less than 1.5-fold of the normal state or a decrease to not more than ⅔ of the normal state, and by the “substantially no variation” is meant an expression level of ⅔ to 1.5-fold of the normal state.

Specifically, as a PLsis marker gene detected by the reagent of the present invention, 54 kinds of genes (including 7 kinds of EST) having the same base sequences or substantially the same base sequences as shown in SEQ ID NO n (wherein n is an odd number between 1 and 57, an integer number between 59 and 63, an even number between 64 and 98, 100 or 101) can be mentioned. As used herein, by the “substantially the same base sequences” is meant base sequences capable of hybridizing to nucleic acids having complementary strand sequences of base sequences shown in SEQ ID NO n (wherein n is an odd number between 1 and 57, an integer number between 59 and 63, an even number between 64 and 98, 100 or 101) under high stringent conditions, wherein the proteins encoded thereby are the same or substantially the same proteins encoded by the base sequences shown in said SEQ ID NOs. The “high stringent conditions” refers to the conditions under which nucleic acids having complementary strand sequences of base sequences shown in SEQ ID NO n (wherein n is an odd number between 1 and 57, an integer number between 59 and 63, an even number between 64 and 98, 100 or 101) can hybridize with nucleic acids having base sequences showing complementarity of not less than about 70%, preferably not less than about 80%, more preferably not less than about 90%, particularly preferably not less than about 95%, in the overlapping regions, and, for example, a sodium concentration of about 19-40 mM, preferably about 19-20 mM, a temperature of about 50-70° C., preferably about 60-65° C., and particularly preferably, a sodium concentration of about 19 mM and a temperature of about 65° C. can be mentioned. Those skilled in this field can easily obtain desired stringency by suitably changing a salt concentration of the hybridization solution, a temperature of hybridization reaction, a probe concentration, a probe length, a mismatch number, a hybridization reaction time, a salt concentration of the washing solution, a washing temperature, and the like.

The “substantially the same protein” refers to a protein having an amino acid sequence showing not less than about 60%, preferably not less than about 70%, more preferably not less than about 80%, particularly preferably not less than about 90%, most preferably not less than about 95%, homology to the amino acid sequence shown in SEQ ID NO m (wherein m is an even number between 2 and 58, or an odd number between 65 and 99) and the same level of activity as the protein having the amino acid sequence shown in the above-mentioned SEQ ID NOs. Alternatively, when a PLsis marker gene has the same or substantially the same base sequence as the base sequence shown in SEQ ID NO n (wherein n is an integer number between 59 and 63, 100 or 101), the “substantially the same protein” refers to a protein having a partial amino acid sequence showing not less than about 60%, preferably not less than about 70%, more preferably not less than about 80%, particularly preferably not less than about 90%, most preferably not less than about 95%, homology to the partial amino acid sequence encoded by the base sequence shown in SEQ ID NO n (wherein n is an integer number between 59 and 63, 100 or 101) and the same level of activity as the protein having the partial amino acid sequence shown in the above-mentioned.

The “same level of activity” means that the activity is qualitatively the same (e.g., physiologically or pharmacologically), and preferably, quantitatively equivalent (e.g., 0.5- to 2-fold), but may be different. As long as the homology conditions of amino acid sequence are satisfied, moreover, other quantitative elements such as molecular weight and the like may be different.

With regard to the amino acid sequence, a “homology” means a proportion (%) of the same amino acid residue and analogous amino acid residue to the whole amino acid residues overlapped in the optimal alignment (preferably, the algorithm is such that a gap can be introduced into one or both of the sequences for an optimal alignment) where two amino acid sequences are aligned using a mathematic algorithm known in the technical field. The “analogous amino acid” means amino acids having similar physiochemical properties, and, for example, the amino acids are classified into groups such as an aromatic amino acid (Phe, Trp, Tyr), an aliphatic amino acid (Ala, Leu, Ile, Val), a polar amino acid (Gln, Asn), a basic amino acid (Lys, Arg, His), an acidic amino acid (Glu, Asp), an amino acid having a hydroxy group (Ser, Thr) and an amino acid having a small side-chain (Gly, Ala, Ser, Thr, Met). Substitution by such analogous amino acids is expected not to change the phenotype of proteins (i.e., conservative amino acid substitution). Specific examples of the conservative amino acid substitution are known in this technical field and described in various literatures (e.g., see Bowie et al., Science, 247: 1306-1310 (1990)).

The homology of the amino acid sequence in the present specification can be calculated using homology calculation algorithm NCBI BLAST (National Center for Biotechnology Information Basic Local Alignment Search Tool) under the following conditions (expectancy=10; allowing gap; matrix=BLOSUM62; filtering=OFF). Other Algorithms to determine a homology of amino acid sequence include, for example, the algorithm as described in Karlin et al., Proc. Natl. Acad. Sci. USA, 90: 5873-5877 (1993) [the algorithm is incorporated into NBLAST and XBLAST programs (version 2.0) (Altschul et al., Nucleic Acids Res., 25: 3389-3402 (1997))], the algorithm as described in Needleman et al., J. Mol. Biol., 48: 444-453 (1970) [the algorithm is incorporated into a GAP program in a GCG software package], the algorithm as described in Myers and Miller, CABIOS, 4: 11-17 (1988) [the algorithm is incorporated into an ALIGN program (version 2.0) which is a part of a CGC sequence alignment software package], the algorithm as described in Pearson et al., Proc. Natl. Acad. Sci. USA, 85: 2444-2448 (1988) [the algorithm is incorporated into a FASTA program in a GCG software package], and the like, which can be preferably used in a similar manner.

More preferably, an amino acid sequence substantially the same as the amino acid sequence shown in SEQ ID NO m (wherein m is an even number between 2 and 58, or an odd number between 65 and 99) is an amino acid sequence having not less than about 60%, preferably not less than about 70%, more preferably not less than about 80%, homology to the amino acid sequence shown in any of said SEQ ID NOs; a partial amino acid sequence substantially the same as the partial amino acid sequence encoded by the base sequence shown in SEQ ID NO n (wherein n is an integer number between 59 and 63, 100 or 101) is a partial amino acid sequence having not less than about 60%, preferably not less than about 70%, more preferably not less than about 80%, homology to the partial amino acid sequence encoded by the base sequence shown in any of said SEQ ID NOs.

The protein having such homology includes, for example, a protein containing 1) an amino acid sequence shown in SEQ ID NO m (wherein m is an even number between 2 and 58, or an odd number between 65 and 99) (or a partial amino acid sequence encoded by the base sequence shown in SEQ ID No n (wherein n is an integer number between 59 and 63, 100 or 101)) wherein one or more (preferably about 1-30, more preferably about 1-10, particularly preferably several (1-5)) amino acids have been deleted, 2) an amino acid sequence shown in SEQ ID NO m (wherein m is an even number between 2 and 58, or an odd number between 65 and 99) (or a partial amino acid sequence encoded by the base sequence shown in SEQ ID No n (wherein n is an integer number between 59 and 63, 100 or 101)) wherein one or more (preferably about 1-30, more preferably about 1-10, particularly preferably several (1-5)) amino acids have been added, 3) an amino acid sequence shown in SEQ ID NO m (wherein m is an even number between 2 and 58, or an odd number between 65 and 99) (or a partial amino acid sequence encoded by the base sequence shown in SEQ ID NO n (wherein n is an integer number between 59 and 63, 100 or 101)) wherein one or more (preferably about 1-30, more preferably about 1-10, particularly preferably several (1-5)) amino acids have been inserted, 4) an amino acid sequence shown in SEQ ID NO m (wherein m is an even number between 2 and 58, or an odd number between 65 and 99) (or a partial amino acid sequence encoded by the base sequence shown in SEQ ID NO n (wherein n is an integer number between 59 and 63, 100 or 101)) wherein one or more (preferably about 1-30, more preferably about 1-10, particularly preferably several (1-5)) amino acids have been substituted by other amino acids, or 5) an amino acid sequence wherein the above-mentioned sequences have been combined, and the like.

When the amino acid sequence is inserted, deleted or substituted as mentioned above, the position of the insertion, deletion or substitution is not particularly limited.

More specifically, as a gene having a substantially the same base sequence as the base sequence shown in SEQ ID NO n (wherein n is an odd number between 1 and 57, an integer number between 59 and 63, an even number between 64 and 98, 100 or 101), allele variants of a rat gene having the base sequence shown in any of these SEQ ID NOs, orthologs of the gene in mammals other than rat (e.g., human, monkey, bovine, horse, swine, sheep, goat, dog, cat, rabbit, hamster, guinea pig, mouse etc.) and the like can be mentioned.

The base sequences of PLsis marker genes from rat of the present invention [i.e., base sequences shown in SEQ ID NO n (wherein n is an odd number between 1 and 57, an integer number between 59 and 63, an even number between 64 and 98, 100 or 101)] are all known, and published in the GenBank database under the accession Nos. of 1) NM_{—}031355, 3) NM_{—}053359, 5) NM_{—}017051, 7) NM_{—}013052, 9) NM_{—}053372, 11) NM_{—}053843, 13) NM_{—}001004202, 15) NM_{—}012512 (or AW916647), 17) NM_{—}019186, 19) NM_{—}024139, 21) XM_{—}215858 (or AA891920), 23) NM_{—}012778 (or L07268), 25) XM_{—}213793 (or AI412863), 27) NM_{—}057114, 29) NM_{—}053800, 31) NM_{—}022391, 33) XM_{—}213921(or BE099979), 35) L38482, 37) NM_{—}001007742 (or BI275880), 39) XM_{—}214451 (or AI600237), 41) XM_{—}226624 (or BI291626), 43) NM_{—}022597, 45) NM_{—}145184, 47) NM_{—}053719, 49) NM_{—}017073, 51) NM_{—}053582, 53) NM_{—}138881, 55) XM_{—}340803 (or BI303656), 57) XM_{—}213333 (or AI171327), 59) AI029175, 60) BM392055, 61) BI288013, 62) AI172141, 63) AI600030, 64) NM_{—}012904, 66) NM_{—}053598, 68) NM_{—}053554, 70) NM_{—}053822, 72) NM_{—}053587, 74) NM_{—}001012345 (or XM_{—}341887), 76) NM_{—}022205, 78) NM_{—}031512, 80) XM_{—}223508, 82) NM_{—}053373, 84) NM_{—}012992, 86) NM_{—}001009717, 88) NM_{—}031010, 90) XM_{—}341053, 92) XM_{—}214152, 94) XM_{—}346274, 96) NM_{—}001013233 (or XM_{—}344660), 98) XM_{—}340780, 100) BM389006 and 101) AA799328, respectively.

Moreover, as human orthologs corresponding to them, in sequence, 1) NM_{—}005662, 3) NM_{—}004045, 5) NM_{—}000636, 7) NM_{—}003405, 9) NM_{—}003064, 11) NM_{—}000569 (NM_{—}000570 as variant), 13) (a human gene corresponding to NM_{—}001004202 is not known), 15) AK026463 (NM_{—}004048 as variant), 17) NM_{—}005738 (NM 212460 as variant), 19) NM_{—}007236, 21) NM_{—}013248, 23) NM_{—}000385 (NM_{—}198098 as variant), 25) NM_{—}016433, 27) NM_{—}002574 (NM_{—}181696, NM_{—}181697 as variant), 29) NM_{—}003229, 31) NM_{—}004219, 33) NM_{—}003851 (similar to both XM_{—}213921 and AI029175), 35) NM_{—}004359, 37) NM_{—}016172, 39) NM_{—}004280, 41) NM_{—}012081, 43) NM_{—}001908 (NM_{—}147780, NM_{—}147781, NM_{—}147782, NM_{—}147783 as variant), 45) NM_{—}006313, 47) NM_{—}198449, 49) NM_{—}002065, 51) NM_{—}022164, 53) NM_{—}080657, 55) NM_{—}024632, 57) NM_{—}152766, 59) NM_{—}003851 (similar to both XM_{—}213921, AI029175), 60) NM_{—}018571, 61) NM_{—}015982, 62) NM_{—}000037 (NM_{—}020475, NM_{—}020476, NM_{—}020477 as variant), 63) (a human gene corresponding to AI600030 is not known), 64) NM_{—}000700, 66) NM_{—}019094 (NM_{—}199040 as variant), 68) NM_{—}001008660 (NM_{—}007166 as variant), 70) NM_{—}002964, 72) NM_{—}002965, 74) NM_{—}032564, 76) NM_{—}001008540 (NM_{—}003467 as variant), 78) NM_{—}000576, 80) NM_{—}012161 (NM_{—}033535 as variant), 82) NM_{—}005091, 84) NM_{—}002520 (NM_{—}199185 as variant), 86) NM_{—}052972, 88) NM_{—}001140, 90) NM_{—}001913 (NM_{—}181500, NM_{—}181552 as variant), 92) NM_{—}005192, 94) NM_{—}007213, 96) NM_{—}002101 (NM_{—}016815 as variant), 98) NM_{—}012075 and 100) NM_{—}003258 (101) a human gene corresponding to AA799328 is not known) can be mentioned.

A gene having the same or substantially the same base sequence as the base sequence shown in SEQ ID NO:1 (hereinafter sometimes to be abbreviated as “vdac3”) encodes a protein at the opening of a voltage-dependent ion channel of mitochondrial outer membrane that possibly functions for the transport of adenine nucleotides (voltage-dependent anion channel 3).

A gene having the same or substantially the same base sequence as the base sequence shown in SEQ ID NO:3 (hereinafter sometimes to be abbreviated as “atox1”) encodes copper binding protein that mediates the intracellular copper transport and homeostasis (ATX1 (antioxidant protein 1) homolog 1 (yeast)).

A gene having the same or substantially the same base sequence as the base sequence shown in SEQ ID NO:5 (hereinafter sometimes to be abbreviated as “sod2”) encodes mitochondrial enzyme that converts superoxide to hydrogen peroxide (superoxide dismutase 2, mitochondrial).

A gene having the same or substantially the same base sequence as the base sequence shown in SEQ ID NO:7 (hereinafter sometimes to be abbreviated as “ywhah”) encodes a protein, which is an apoptosis inhibitor and activator of tyrosine hydroxylase and tryptophan hydroxylase (tyrosine 3-monooxygenase/tryptophan 5-monooxygenase activation protein, eta polypeptide).

A gene having the same or substantially the same base sequence as the base sequence shown in SEQ ID NO:9 (hereinafter sometimes to be abbreviated as “slpi”) encodes an inhibitor of a protease such as trypsin, cathepsin G and neutrophil elastase and the like (secretory leukocyte protease inhibitor).

A gene having the same or substantially the same base sequence as the base sequence shown in SEQ ID NO:11 (hereinafter sometimes to be abbreviated as “fcgr3”) encodes a protein, which is bound with immunoglobulin and initiates various immune responses (Fc receptor, IgG, low affinity III).

A gene having the same or substantially the same base sequence as the base sequence shown in SEQ ID NO:13 (hereinafter sometimes to be abbreviated as “ccl6”) encodes CC chemokine that promotes migration and humectant of inflammatory cell in rat (chemokine (C-C motif) ligand 6). However, the corresponding human gene is not known.

A gene having the same or substantially the same base sequence as the base sequence shown in SEQ ID NO:15 (hereinafter sometimes to be abbreviated as “B2m”) encodes a component of MHC class I antigen that acts on transepithelial transport of IgG (Beta-2 microglobulin).

A gene having the same or substantially the same base sequence as the base sequence shown in SEQ ID NO:17% (hereinafter sometimes to be abbreviated as “arl4”) encodes GTP binding protein that plays a key role in the vesicular transport and protein secretion (ADP-ribosylation-like 4).

A gene having the same or substantially the same base sequence as the base sequence shown in SEQ ID NO:19 (hereinafter sometimes to be abbreviated as “chp”) encodes Ca²⁺ binding protein that plays a role in the membrane transport (calcium binding protein p22).

A gene having the same or substantially the same base sequence as the base sequence shown in SEQ ID NO:21 encodes a protein similar to NXT1, an NTF2-related transport protein (Similar to NTF2-related export protein NXT1 (LOC296219), mRNA).

A gene having the same or substantially the same base sequence as the base sequence shown in SEQ ID NO:23 (hereinafter sometimes to be abbreviated as “aqp1”) encodes a protein constituting a water channel (aquaporin 1 (channel-forming integral protein, 28 kDa)).

A gene having the same or substantially the same base sequence as the base sequence shown in SEQ ID NO:25 encodes a protein similar to a glycolipid transfer protein, which is involved in the intermembrane transport of glycosphingolipid, glycoglycerolipid (Similar to Glycolipid transfer protein (LOC288707), mRNA).

A gene having the same or substantially the same base sequence as the base sequence shown in SEQ ID NO:27 (hereinafter sometimes to be abbreviated as “prdx1”) encodes an antioxidant having peroxydase activity induced by oxidative stress (peroxiredoxin 1).

A gene having the same or substantially the same base sequence as the base sequence shown in SEQ ID NO:29 (hereinafter sometimes to be abbreviated as “txn”) encodes a protein involved in the response to UV and oxidative stress, which decreases a reactive oxygen intermediate (thioredoxin).

A gene having the same or substantially the same base sequence as the base sequence shown in SEQ ID NO:31 (hereinafter sometimes to be abbreviated as “pttg”) is a premalignant gene, which is downregulated in the response to serum starvation, and promotes cell growth and angiogenesis (pituitary tumor-transforming 1).

A gene having the same or substantially the same base sequence as the base sequence shown in SEQ ID NO:33 encodes a protein similar to cellular repressor CREG of E1A-stimulated gene involved in transcriptional regulation relating to cell growth or differentiation (Similar to cellular repressor of E1A-stimulated genes CREG (LOC289185), mRNA).

A gene having the same or substantially the same base sequence as the base sequence shown in SEQ ID NO:35 encodes an enzyme, which is conjugated with ubiquitin, and regulates transfer from G1 phase to S phase and chromosome alignment (similar to cell division cycle 34; ubiquitin-conjugating enzyme E2-32 KDA complementing; ubiquitin carrier protein; ubiquitin-protein ligase (LOC299602), mRNA).

A gene having the same or substantially the same base sequence as the base sequence shown in SEQ ID NO:37 (hereinafter sometimes to be abbreviated as “ubadc1”) encodes a protein assumed to be an intracellular signal molecule capable of regulating the growth of endothelial cell (ubiquitin associated domain containing 1).

A gene having the same or substantially the same base sequence as the base sequence shown in SEQ ID NO:39 encodes a protein similar to eukaryotic translation elongation factor 1ε1 (similar to eukaryotic translation elongation factor 1 epsilon 1 (LOC291057), mRNA).

A gene (EST) having the same or substantially the same base sequence as the base sequence shown in SEQ ID NO:41 encodes a protein similar to RNA polymerase II elongation factor ELL2 (Similar to RNA polymerase II elongation factor ELL2 (LOC309918), mRNA).

A gene having the same or substantially the same base sequence as the base sequence shown in SEQ ID NO:43 (hereinafter sometimes to be abbreviated as “ctsb”) encodes cysteine protease belonging to the papain family derived from lysosome (cathepsin B).

A gene having the same or substantially the same base sequence as the base sequence shown in SEQ ID NO:45 (hereinafter sometimes to be abbreviated as “usp15”) encodes an enzyme belonging to the ubiquitin specific cysteine protease family (ubiquitin specific protease 15).

A gene having the same or substantially the same base sequence as the base sequence shown in SEQ ID NO:47 (hereinafter sometimes to be abbreviated as “emb”) encodes a transmembrane protein, which is a cell adhesion molecule (embigin).

A gene having the same or substantially the same base sequence as the base sequence shown in SEQ ID NO:49 (hereinafter sometimes to be abbreviated as “glu1”) encodes glutamine synthetase (glutamine synthetase 1).

A gene having the same or substantially the same base sequence as the base sequence shown in SEQ ID NO:51 (hereinafter sometimes to be abbreviated as “lcn7”) encodes cathepsin B associated protein inactive as a catalyst, which is assumed to be an extracellular matrix protein (lipocalin 7).

A gene having the same or substantially the same base sequence as the base sequence shown in SEQ ID NO:53 (hereinafter sometimes to be abbreviated as “best5”) encodes an antivirus protein, which is induced by interferons (Best5 protein).

A gene having the same or substantially the same base sequence as the base sequence shown in SEQ ID NO:55 encodes a protein similar to a transcription corepressor Sin3A-associated protein (SAP30) related to histone deacetylase (Similar to Sin3A associated protein p30-like).

A gene having the same or substantially the same base sequence as the base sequence shown in SEQ ID NO:57 encodes a protein similar to hypothetical protein MGC40107 (Similar to hypothetical protein MGC40107 (LOC287442), mRNA).

A gene (EST) having the same or substantially the same base sequence as the base sequence shown in SEQ ID NO:59 has a sequence similar to mRNA of cellular repressor CREG of a gene stimulated by E1A involved in the transcriptional regulation relating to the cell growth or differentiation (Similar to cellular repressor of E1A-stimulated genes CREG (LOC289185), mRNA).

A gene (EST) having the same or substantially the same base sequence as the base sequence shown in SEQ ID NO:60 has a sequence similar to mRNA of an antiapoptotic protein, which potentiates the activation of JNK1 (Similar to amyotrophic lateral sclerosis 2 (juvenile) chromosome region, candidate 2).

A gene (EST) having the same or substantially the same base sequence as the base sequence shown in SEQ ID NO:61 has a sequence similar to mRNA of RNA binding protein involved in the translational repression of mRNA in germ cell (Similar to germ cell specific Y-box binding protein).

A gene (EST) having the same or substantially the same base sequence as the base sequence shown in SEQ ID NO:62 has a sequence similar to mRNA of cytoskeletal anchoring protein Ankyrin1 that adheres cytoskeletal element to a plasma membrane (Similar to Ankyrin 1).

The function of a gene (EST) having the same or substantially the same base sequence as the base sequence shown in SEQ ID NO:63 is unknown.

A gene having the same or substantially the same base sequence as the base sequence shown in SEQ ID NO:64 (hereinafter sometimes to be abbreviated as “anxa1”) encodes calcium-dependent phospholipid binding protein that inhibits phospholipase A2 (annexinA1).

A gene having the same or substantially the same base sequence as the base sequence shown in SEQ ID NO:66 (hereinafter sometimes to be abbreviated as “nudt4”) encodes diphosphoinositol polyphosphate phosphohydrolase possibly involved in the metabolism of inositol phosphate (nudix (nucleoside diphosphate linked moiety X)-type motif 4).

A gene having the same or substantially the same base sequence as the base sequence shown in SEQ ID NO:68 (hereinafter sometimes to be abbreviated as “picalm”) encodes a protein, which is bound with a clathrin heavy chain and acts on endocytosis (Phosphatidylinositol binding clathrin assembly protein).

A gene having the same or substantially the same base sequence as the base sequence shown in SEQ ID NO:70 (hereinafter sometimes to be abbreviated as “S100a8”) encodes a protein, which is a member of the S100 family, forms a complex with S100A9, and mediates arachidonic acid secretion and leukocyte supplementation to a site of inflammation (S100 calcium binding protein A8 (calgranulin A)).

A gene having the same or substantially the same base sequence as the base sequence shown in SEQ ID NO:72 (hereinafter sometimes to be abbreviated as “S100a9”) encodes a protein, which is a member of the S100 family, forms a complex with S100A8, and mediates arachidonic acid secretion and leukocyte supplementation to a site of inflammation (S100 calcium binding protein A9 (calgranulin B)).

A gene having the same or substantially the same base sequence as the base sequence shown in SEQ ID NO:74 (hereinafter sometimes to be abbreviated as “dgat2”) encodes a member of the diacylglycerol acyltransferase family, which catalyzes triacylglycerol biosynthesis (Diacylglycerol O-acyltransferase homolog 2 (mouse)).

A gene having the same or substantially the same base sequence as the base sequence shown in SEQ ID NO:76 (hereinafter sometimes to be abbreviated as “cxcr4”) encodes GPCR, which is bound with CXC chemokine (Chemokine (C-X-C motif) receptor 4).

A gene having the same or substantially the same base sequence as the base sequence shown in SEQ ID NO:78 (hereinafter sometimes to be abbreviated as “il1b”) encodes cytokine that regulates defense reaction and inflammation reaction (interleukin 1 beta).

A gene having the same or substantially the same base sequence as the base sequence shown in SEQ ID NO:80 (hereinafter sometimes to be abbreviated as “fbxl5”) encodes assumed subunit of SCF ubiquitin ligase involved in proteolysis (F-box and leucine-rich repeat protein 5 (predicted)).

A gene having the same or substantially the same base sequence as the base sequence shown in SEQ ID NO:82 (hereinafter sometimes to be abbreviated as “pglyrp1”) encodes a protein, which is bound with peptidoglycan and gram-positive bacterium and is involved in congenital immunity (peptidoglycan recognition protein 1).

A gene having the same or substantially the same base sequence as the base sequence shown in SEQ ID NO:84 (hereinafter sometimes to be abbreviated as “npm1”) encodes a protein, which is a nucleic acid binding ribonuclease possibly involved in ribosome assembly and is a molecular chaperon (nucleophosmin 1).

A gene having the same or substantially the same base sequence as the base sequence shown in SEQ ID NO:86 encodes a serum protein (Similar to leucine-rich alpha-2-glycoprotein) containing plural leucine-rich repeats.

A gene having the same or substantially the same base sequence as the base sequence shown in SEQ ID NO:88 (hereinafter sometimes to be abbreviated as “alox15”) encodes an enzyme that converts arachidonic acid to 15-hydroperoxyeicosatetraenoic acid and lipoxin A4 (arachidonate 12-lipoxygenase).

A gene having the same or substantially the same base sequence as the base sequence shown in SEQ ID NO:90 (hereinafter sometimes to be abbreviated as “cutl1”) encodes a protein (Cut-like 1 (Drosophila)) that acts on the cell cycle of mitosis, and possibly regulates an immune response to apoptosis-inducing stimulation.

A gene having the same or substantially the same base sequence as the base sequence shown in SEQ ID NO:92 encodes tyrosine-serine phosphatase that inhibits the progression of cell cycle (cyclin-dependent kinase inhibitor 3 (predicted)).

A gene having the same or substantially the same base sequence as the base sequence shown in SEQ ID NO:94 encodes a protein, which is a member of the prenylated Rab acceptor (PRA1) family and has moderate similarity to a glutamic acid transporter (Similar to DXImx39e protein).

A gene having the same or substantially the same base sequence as the base sequence shown in SEQ ID NO:96 encodes an erythrocyte transmembrane sialoglycoprotein, which forms a triple complex with Epb4.1 and Mpp1 and possibly plays a role in the regulation of cell shape (Similar to glycophorin C isoform 2).

A gene having the same or substantially the same base sequence as the base sequence shown in SEQ ID NO:98 encodes a member of uncharacterized protein UPF0171 family (similar to CGTHBA protein (−14 gene protein) (predicted)).

A gene (EST) having the same or substantially the same base sequence as the base sequence shown in SEQ ID NO:100 has a sequence similar to mRNA of cytoplasmic enzyme that synthesizes thymidine acid for DNA synthesis (similar to thymidine kinase 1).

The function of a gene (EST) having the same or substantially the same base sequence as the base sequence shown in SEQ ID NO:101 is unknown.

The genes having the same or substantially the same base sequence as the base sequences shown in SEQ ID NOs: 1, 3, 5, 7, 9, 11, 13, 15, 64, 66, 68, 70, 72, 74, 76, 78, 80, 82, 84 and 86 show increased in vivo expression in correlation with PLsis expression in some organ (see Example 2, Table 3 and Example 4, Table 7 to be described below), and the genes having the same or substantially the same base sequence as the base sequences shown in SEQ ID NOs: 17, 19, 21, 23, 25, 27, 29, 31, 33, 35, 37, 39, 41, 43, 45, 47, 49, 51, 53, 55, 57, 59-63, 88, 90, 92, 94, 96, 98, 100 and 101 (see Example 2, Table 4 and Example 4, Table 8 to be described below) show decreased expression in correlation with PLsis expression.

As a nucleic acid capable of detecting the expression of a PLsis marker gene contained in the reagent for determining PLsis of the present invention (hereinafter sometimes to be abbreviated as “the reagent of the present invention”), for example, a nucleic acid (probe) capable of hybridizing to a transcription product of a PLsis marker gene, an oligonucleotide set capable of functioning as a primer amplifying a part or whole of the transcription product and the like can be mentioned. That is, as the nucleic acid, for example, a nucleic acid capable of hybridizing to a nucleic acid having a base sequence shown by SEQ ID NO n (wherein n is any of an odd number between 1 and 57, an integer number between 59 and 63, an even number between 64 and 98, 100 or 101) (sense strand=coding strand) under high stringent conditions, and/or a nucleic acid capable of hybridizing to a nucleic acid having a base sequence complementary to the base sequence (antisense strand=non-coding strand) under high stringent conditions can be preferably mentioned. Being “capable of hybridizing under high stringent conditions” means as defined above. The nucleic acid may be a DNA or RNA, or a DNA/RNA chimera. Preferably, DNA can be mentioned.

The nucleic acid to, be used as a probe may be double strand or single strand. In the case of a double-strand, it may be a double-strand DNA, a double-strand RNA or a DNA:RNA hybrid. In the case of a single strand, a sense strand (e.g., in case of cDNA, cRNA) or an antisense strand (e.g., in case of mRNA, cDNA) can be selected for use according to the sample to be used. The length of the nucleic acid is not particularly limited as long as it can specifically hybridize to a target nucleic acid and, for example, it is not less than about 15 bases, preferably not less than about 30 bases. The nucleic acid is preferably labeled with a labeling agent to enable detection or quantitation of a target nucleic acid.

As examples of the labeling agent, a radioisotope, an enzyme, a fluorescent substance, a luminescent substance and the like can be used. Examples of the radioisotope include [³²P], [³H], [¹⁴C] and the like. As the enzyme, those that are stable and high in specific activity are preferred; for example, β-galactosidase, β-glucosidase, alkaline phosphatase, peroxidase, malate dehydrogenase and the like can be used. As examples of the fluorescent substance, fluorescamine, fluorescein isothiocyanate and the like can be used. As examples of the luminescent substance, luminol, luminol derivative, luciferin, lucigenin and the like can be used. Furthermore, a biotin-(strepto)avidin system can also be used for binding of a probe with a labeling agent. For immobilization of a nucleic acid to be a probe on a solid phase, a nucleic acid in a sample can be labeled using a labeling agent similar to those mentioned above.

An oligonucleotide set to be used as a primer is not particularly limited as long as it can specifically hybridize to each of a base sequence shown in each SEQ ID NO (sense strand) and a base sequence complementary thereto (antisense strand), and amplify a DNA fragment sandwiched therebetween and, for example, a set of oligo DNAs designed to have a length of about 15 to about 100 bases, preferably about 15 to about 50 bases, and amplify about 100 by to several kbp DNA fragments can be mentioned.

For quantitative analysis of PLsis marker gene expression using a trace amount of an RNA sample, competitive RT-PCR or real-time RT-PCR is preferably used. Competitive RT-PCR is a method for calculating the amount of an object DNA by carrying out a competitive amplification reaction in a reaction mixture in the co-presence of a known amount of other template nucleic acid, which can be amplified by a primer set capable of amplifying the object DNA as a competitor, and comparing the amounts of the amplification products. When using competitive RT-PCR, therefore, the reagent of the present invention can further contain, besides the above-mentioned primer set, a nucleic acid which is amplified by the primer set to produce an amplification product distinguishable from the object. DNA (e.g., amplification product different from the object DNA in size, amplification product showing different migration pattern by a restriction enzyme treatment and the like). This competitor nucleic acid may be a DNA or an RNA. In the case of a DNA, cDNA is synthesized from an RNA sample by a reverse transcription reaction and a competitor is added to perform PCR, and in the case of an RNA, it may be added to an RNA sample from the start to perform RT-PCR. In the latter case, an absolute amount of the original mRNA can also be assumed since the efficiency of the reverse transcription reaction is taken into consideration.

On the other hand, real-time RT-PCR does not require electrophoresis, since the amplification amount by PCR can be monitored real-time, and the expression of a PLsis marker gene can be analyzed more rapidly. Generally, monitoring is performed using various fluorescent reagents. These include reagents (intercalator) emitting fluorescence by binding to double stranded DNA such as SYBR Green I, ethidium bromide and the like, nucleic acids usable as the above-mentioned probes (the nucleic acid hybridizes to the target nucleic acid within amplification region), wherein the both ends are respectively modified with a fluorescent substance (e.g., FAM, HEX, TET, FITC etc.) and a quenching substance (e.g., TAMRA, DABCYL etc.) and the like.

The nucleic acid functionable as a probe capable of detecting the expression of a PLsis marker gene can be obtained by amplifying a nucleic acid having a desired length by PCR using the above-mentioned primer set capable of amplifying a part or whole of a transcription product of the gene and using cDNA or genomic DNA from any cell (e.g., hepatocyte, splenocyte, nerve cell, glial cell, pancreatic β cell, myelocyte, mesangial cell, Langerhans' cell, epidermal cell, epithelial cell, goblet cell, endothelial cell, smooth muscle cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, or interstitial cell, or corresponding precursor cell, stem cell or cancer cell thereof, and the like) of a mammal (e.g., human, monkey, bovine, horse, swine, sheep, goat, dog, cat, rabbit, hamster, guinea pig, mouse, rat and the like), or any tissue where such cells are present (e.g., brain, any portion of the brain (e.g., olfactory bulb, amygdaloid nucleus, basal ganglia, hippocampus, thalamus, hypothalamus, cerebral cortex, medulla oblongata, cerebellum), spinal cord, hypophysis, stomach, pancreas, kidney, liver, gonad, thyroid, gall-bladder, bone marrow, adrenal gland, skin, lung, gastrointestinal tract (e.g., large intestine, small intestine), blood vessel, heart, thymus, spleen, submandibular gland, peripheral blood, prostate, testicle, ovary, placenta, uterus, bone, joint, adipose tissue, skeletal muscle, and the like) as a template, or cloning the above-mentioned PLsis marker gene or cDNA from cDNA or genomic DNA library derived from the aforementioned cell or tissue by colony or plaque hybridization and the like and, where necessary, treating them with a restriction enzyme and the like to give a fragment having a suitable length. The hybridization can be carried out according to the method described in, for example, Molecular Cloning, 2nd. ed. (mentioned above) and the like. When a commercially available library is used, hybridization can be carried out according to the method described in the instruction manual attached to the library. Alternatively, the nucleic acid can also be obtained by chemically synthesizing a part or whole of the base sequence and/or its complementary strand sequence based on the information of a base sequences shown in SEQ ID NO n (wherein n is an odd number between 1 and 57, an integer number between 59 and 63, an even number between 64 and 98, 100 or 101) using a commercially available DNA/RNA automatic synthesizer and the like. In addition, a chip with a solid phased nucleic acid can also be prepared by direct in situ (on chip) synthesis of the nucleic acid on a solid phase such as silicone, glass and the like.

The nucleic acid functionable as a primer capable of amplifying a part or whole of a transcription product of a PLsis marker gene can be obtained by chemically synthesizing a part of a base sequence and its complementary strand sequence, based on the information of the base sequences shown in SEQ ID NO n (wherein n is an odd number between 1 and 57, an integer number between 59 and 63, an even number between 64 and 98, 100 or 101), using a commercially available DNA/RNA automatic synthesizer and the like.

The nucleic acid capable of detecting the expression of a PLsis marker gene can be provided as a solid in a dry state or in the form of an alcohol precipitate, or in a dissolution state in water or a suitable buffer (e.g., TE buffer etc.). When used as a labeled probe, the nucleic acid can be provided after labeling with any of the above-mentioned labeling substances, or provided independently of a labeling substance and labeled when in use.

Alternatively, the nucleic acid can be immobilized on a suitable solid phase. The solid phase is exemplified by, but not limited to, glass, silicone, plastic, nitrocellulose, nylon, polyvinylidene difluoride and the like. The immobilizing means is exemplified by, but not limited to, a method comprising previously introducing a functional group such as amino group, aldehyde group, SH group, biotin and the like into a nucleic acid, introducing, onto a solid phase, a functional group (e.g., aldehyde group, amino group, SH group, streptavidin and the like) reactive with the nucleic acid, and crosslinking the solid phase and the nucleic acid with covalent bond between these functional groups, or immobilizing a polyanionic nucleic acid with electrostatic bond using a polycation-coated solid phase and the like.

One preferable embodiment of a nucleic acid probe immobilized on a solid phase is ArrayPlate™ etc. provided by High Throughput Genomics, Inc. ArrayPlate™ is a 96 well plate immobilized with various nucleic acid probes regularly disposed in the bottom of each well (e.g., 4×4 array). By the presence of a nucleic acid having one end hybridizable to a probe and the other end hybridizable to a target nucleic acid as a mediating spacer, a hybridization reaction of the probe with a target nucleic acid can be carried out in a liquid phase rather than on the solid phase surface, which enables a quantitative measurement of the target nucleic acid. Accordingly, expression variation of various PLsis marker genes can be simultaneously detected at once in a single well, and once sufficient quantitation performance is achieved, an advantage of higher efficiency than real-time PCR wherein expression variation in each marker gene is individually detected can be afforded.

The reagent of the present invention can further contain, in addition to a nucleic acid capable of detecting the expression of a PLsis marker gene, other substance necessary for the reaction for detecting the expression of the gene, which does not interfere with the reaction when preserved in coexistence. Alternatively, the reagent of the present invention can also be provided as a kit together with a separate reagent containing other substance necessary for the reaction for detecting the expression of a PLsis marker gene. For example, when the reaction for detecting the expression of a PLsis marker gene is PCR, as said other substance, for example, a reaction buffer, dNTPs, a thermostable DNA polymerase and the like can be mentioned. When competitive PCR or real-time PCR is used, a competitor nucleic acid, a fluorescent reagent (the above-mentioned intercalator, fluorescence probe etc.) and the like can be further contained.

In drug-induced PLsis, for example, it is ideal that each PLsis marker gene should show expression variation for any PLsis-inducing compound and substantially no expression variation for any PLsis non-inducing compound, though, in fact, such results cannot be obtained all the time. Therefore, when expression of each marker gene is used as a single index, emergence of a certain extent of false-positive and false-negative compounds is generally inevitable. However, by examining expression variation of plural PLsis marker genes, the determination accuracy can be further improved.

Accordingly, the present invention also provides a kit for determining PLsis, which contains two or more reagents containing a nucleic acid capable of detecting a PLsis marker gene in combination. The nucleic acid to be contained in each reagent can detect PLsis marker genes different from each other. Moreover, at least one of the reagents contains the above-mentioned reagent of the present invention, namely, a nucleic acid capable of detecting a gene having a base sequence the same or substantially the same as the base sequence shown by any of SEQ ID NO n (wherein n is an odd number between 1 and 57, an integer number between 59 and 63, an even number between 64 and 98, 100 or 101). More preferably, a kit that detects any 2 or more, further preferably 3 or more, still more preferably 4 or more, particularly preferably 5 or more, and most preferably 6 or more, of the above-mentioned 54 PLsis marker genes can be mentioned.

As the nucleic acid capable of detecting a PLsis marker gene possibly contained in a reagent other than the reagent of the present invention, which is contained as the constituent member of the kit of the present invention, is not particularly limited and, for example, a nucleic acid capable of detecting a PLsis marker gene other than the above-mentioned 54 PLsis marker genes, such as a gene encoding a lysosomal enzyme, a gene encoding a lipid metabolism (e.g., cholesterol synthesis, fatty acid elongation, unsaturated fatty acid synthesis etc.)-related protein, a gene encoding a transport (e.g., fatty acid transport, protein transport, amino acid transport etc.)-related protein, a gene encoding a cell growth-related protein, a gene encoding a protease or protease inhibitor, a gene encoding an amino acid metabolism-related protein and the like can be mentioned. More specifically, a nucleic acid capable of detecting human genes having base sequences registered in the GenBank database under the ID Nos. of NM_{—}000859, AL518627, NM_{—}002130, AA639705, BC005807, AF116616, NM_{—}025225, D80010, NM_{—}001731, AW134535, NM_{—}004354, AF135266, AC007182, NM_{—}003832, NM_{—}019058, AB040875, AA488687, NM_{—}018687, NM_{—}021158, BG231932, NM_{—}000235, AA873600, AF096304, AW150953, NM_{—}001360, AC001305, NM_{—}024090, NM_{—}006214, NM_{—}024108, NM_{—}021980, AF003934, NM_{—}000596, U15979, M92934, NM_{—}002087, AK023348, NM_{—}002773, NM_{—}000131, BC003169, NM_{—}002217, NM_{—}003122, NM_{—}001673, NM_{—}000050, U08024, NM_{—}003167, BC005161, AF162690, AW517464, AF116616, NM_{—}017983, NM_{—}016061, BE966922, BE552428, NM_{—}012445, NM_{—}000792, NM_{—}015930, NM_{—}021800, NM_{—}005980, NM_{—}000565, AB033025, AL110298, NM_{—}006931, NM_{—}001955, NM_{—}003897, AA778684, NM_{—}001283, NM_{—}012242, AI934469, NM_{—}003186 and NM_{—}002450 (see above Sawada, H. et al., Toxicol. Sci., 83: 282-92 (2005)), orthologues thereof in other mammals and the like can be mentioned.

The nucleic acid contained in each reagent constituting the kit is particularly preferably so constructed as to detect expression of a PLsis marker gene by the same method (e.g., northern blot, dot blot, DNA array technique, quantitative RT-PCR etc.).

The constitution of the kit of the present invention is exemplified by, but not limited to, one wherein the above-mentioned reagents are separately provided [e.g., when nucleic acid functions as a labeled probe (particularly dot blot analysis), a primer for PCR (particularly real-time quantitative PCR) etc.], one wherein nucleic acids capable of detecting the expression of different PLsis marker genes are contained in a single reagent [e.g., when nucleic acid functions as PCR (particularly, when each marker gene can be distinguished by the size of an amplification product and the like), a labeled probe (particularly, when each marker gene can be distinguished by the size of a transcription product by northern blot analysis) and the like], one wherein nucleic acids capable of detecting the expression of different PLsis marker genes are immobilized in separate regions of a single solid phase [e.g., when nucleic acid functions as a probe for hybridization to label cRNA etc., and the like] and the like.

The present invention also provides a method for determining PLsis in a mammal, comprising detecting expression variation of one or more genes showing varying expression in correlation with PLsis expression in a sample from the mammal. Examples of the mammal include, but not limited to, human, monkey, bovine, horse, swine, sheep, goat, dog, cat, rabbit, hamster, guinea pig, mouse, rat and the like. While preferable animal species can be appropriately selected depending on the object, for example, when the PLsis-inducibility of a drug candidate compound is to be evaluated, human is preferably used in the clinical stage, and monkey, rat, mouse, dog and the like are preferably used in the preclinical stage.

As PLsis in mammals, drug-induced PLsis (encompassing any embodiment such as PLsis caused by administration of a pharmaceutical agent or a veterinary drug or a candidate compound thereof, PLsis caused by accidental ingestion of toxin or exposure to a chemical substance present in the environment etc.), PLsis associated with hereditary phospholipid dysbolism, other disease and the like can be mentioned. Thus, as a mammal to be the test target in the method of the present invention, a test subject (experimental animal) to whom a pharmaceutical agent (or veterinary drug) candidate compound is to be administered, a patient (patient animal) to whom a pharmaceutical agent (or veterinary drug) possibly expressing PLsis has been administered, human or other mammal (possibly) exposed to a chemical substance and the like capable of inducing PLsis and the like, a mammal affected or possibly affected (in the future) with a hereditary or nonhereditary disease possibly inducing PLsis and the like can be mentioned.

A method of exposing a mammal to a test compound for determination of a drug-induced PLsis by a pharmaceutical agent (or veterinary drug) candidate compound is not particularly limited. For example, the test compound can be administered in the form of a solid, a semi-solid, a liquid, an aerosol and the like orally or parenterally (e.g., intravenously, intramuscularly, intraperitoneally, intraarterially, subcutaneously, intradermally, airway etc.). The dose of the test compound varies depending on the kind of the compound, animal species, body weight, administration form and the like and, for example, an amount within the range where the animal can survive and necessary for the target cell to be exposed to the highest concentration of the test compound, at which the cell can survive, for more than a given time and the like can be mentioned. The dose can be administered in one to several portions. While the time from the administration to the sample collection varies depending on the kind of a marker gene used, the in vivo kinetics of the test compound and the like, in general, it is appropriately selected from the range of about 3 hr to about 8 weeks, preferably about 2 to about 14 days from first dose.

As a sample from a mammalian, any cell of a mammal (e.g., human, monkey, bovine, horse, swine, sheep, goat, dog, cat, rabbit, hamster, guinea pig, mouse, rat and the like), (e.g., hepatocyte, splenocyte, nerve cell, glial cell, pancreatic β cell, myelocyte, mesangial cell, Langerhans' cell, epidermal cell, epithelial cell, goblet cell, endothelial cell, smooth muscle cell, fibroblast, fibrocyte, myocyte, adipocyte, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, interstitial cell, or corresponding precursor cell, stem cell or cancer cell thereof, and the like), or any tissue where such cells are present (e.g., brain, any portion of the brain (e.g., olfactory bulb, amygdaloid nucleus, basal ganglia, hippocampus, thalamus, hypothalamus, cerebral cortex, medulla oblongata, cerebellum), spinal cord, eyeball, hypophysis, stomach, pancreas, kidney, liver, gonad, thyroid, gall-bladder, bone marrow, adrenal gland, skin, lung, gastrointestinal tract (e.g., large intestine, small intestine), blood vessel, heart, thymus, spleen, submandibular gland, peripheral blood, prostate, testicle, ovary, placenta, uterus, bone, joint, adipose tissue, skeletal muscle, and the like), and the like can be mentioned. Blood (e.g., peripheral blood), lymphocyte and the like are particularly preferable since rapid and convenient collection is possible, it is less-invasive to animals, and the like.

The PLsis marker gene whose expression variation is examined by the determination method of the present invention is not particularly limited as long as at least one of them has a base sequence the same or substantially the same as the base sequence shown by any of SEQ ID NO: n (wherein n is an odd number between 1 and 57, an integer number between 59 and 63, an even number between 64 and 98, 100 or 101). Preferably, a method for using any two or more, preferably three or more, more preferably four or more, particularly preferably five or more, most preferably six or more, of the above-mentioned 54 PLsis marker genes as detection target can be mentioned.

As the PLsis marker gene other than the above-mentioned 54 genes which can be the detection target in the determination method of the present invention, for example, a gene encoding a lysosomal enzyme, a gene encoding a lipid metabolism (e.g., cholesterol synthesis, fatty acid elongation, unsaturated fatty acid synthesis etc.)-related protein, a gene encoding a transport (e.g., fatty acid transport, protein transport, amino acid transport etc.)-related protein, a gene encoding a cell growth-related protein, a gene encoding a protease or protease inhibitor, a gene encoding an amino acid metabolism-related protein and the like can be mentioned. More specifically, human genes having base sequences registered in the GenBank database under the ID Nos. of NM_{—}014960, NM_{—}000859, AL518627, NM_{—}002130, AA639705, BC005807, AF116616, NM_{—}025225, U47674, D80010, NM_{—}001731, AW134535, NM_{—}004354, AF135266, AC007182, NM_{—}003832, NM_{—}019058, AB040875, AA488687, NM_{—}018687, NM_{—}021158, BG231932, NM_{—}024307, NM_{—}000235, AA873600, D63807, AF096304, AW150953, NM_{—}001360, NM_{—}021969, AC001305, NM_{—}024090, NM_{—}001443, NM_{—}006214, NM_{—}024108, NM_{—}021980, NM_{—}002151, AF003934, NM_{—}000596, U15979, M92934, NM_{—}002087, AK023348, NM_{—}002773, NM_{—}000131, BC003169, NM_{—}002217, NM_{—}003122, NM_{—}001673, NM_{—}000050, NM_{—}001085, U08024, NM_{—}003167, BC005161, AF162690, AW517464, AF116616, NM_{—}017983, AL136653, NM_{—}016061, BE966922, BE552428, NM_{—}022823, NM_{—}012445, NM_{—}000792, NM_{—}015930, NM_{—}021800, NM_{—}005980, NM_{—}000565, AB033025, NM_{—}006931, AL110298, NM_{—}006931, NM_{—}001955, NM_{—}003897, NM_{—}003186, AA778684, NM_{—}001283, NM_{—}012242, AI934469, NM_{—}003186 and NM_{—}002450 (see above Sawada, H. et al., Toxicol. Sci., 83: 282-92 (2005)), orthologues thereof in other mammals and the like can be mentioned.

The expression of a PLsis marker gene in a sample from a mammal can be examined by preparing an RNA (e.g., total RNA, mRNA) fraction from the sample, and detecting a transcription product of the marker gene contained in the fraction. An RNA fraction can be prepared by a known means such as guanidine-CsCl ultracentrifugation method, AGPC method and the like. A high purity total RNA can be prepared rapidly and conveniently from a trace sample using a commercially available RNA extraction kit (e.g., RNeasy Mini Kit; manufactured by QIAGEN etc.). As a means for detecting a transcription product of a PLsis marker gene in an RNA fraction, for example, a method using hybridization (northern blot, dot blot, DNA chip analysis etc.), a method using PCR (RT-PCR, competitive PCR, real-time PCR etc.) and the like can be mentioned. Since expression variation of a PLsis marker gene can be detected rapidly and conveniently with high quantitation performance from a trace sample, quantitative PCR such as competitive PCR, real-time PCR and the like is preferable, and since expression variation of multiple marker genes can be detected at once and quantitation performance can be improved by the selection of a detection method and the like, DNA chip analysis is preferable.

In the case of northern blot or dot blot hybridization, a PLsis marker gene can be detected using the above-mentioned reagent or kit of the present invention containing a nucleic acid to be used as a labeled probe. In the case of Northern hybridization, therefore, an RNA fraction prepared as mentioned above is separated by gel electrophoresis, transferred to a membrane such as nitrocellulose, nylon, polyvinylidene difluoride and the like, hybridized under the above-mentioned “high stringent conditions” in a hybridization buffer containing the reagent of the present invention or each reagent contained in the kit of the present invention, and the label amount bonded to the membrane by a suitable method is measured for each band, whereby the expression amount of each PLsis marker gene can be measured. In the case of dot blot, too, a membrane spotted with an RNA fraction is subjected to a hybridization reaction in the same manner (conducted for each PLsis marker gene), and the label amount of the spot is measured, whereby the expression amount of each marker gene can be measured.

In the case of DNA chip analysis (solid phased probe described for the above-mentioned reagent of the present invention), for example, cDNA harboring a suitable promoter such as T7 promoter and the like is synthesized by reverse transcription reaction from the RNA fraction prepared as mentioned above, and cRNA is synthesized using an RNA polymerase (where labeled cRNA is obtained by using, as a substrate, a mononucleotide labeled with biotin and the like). The labeled cRNA is contacted with the above-mentioned solid phased probe to allow hybridization reaction, and the label amount bonded to each probe on the solid phase is measured, whereby the expression amount of each PLsis marker gene can be measured. This method is advantageous in terms of rapidness and convenience as the number of PLsis marker genes (i.e., probes to be solid phased) to be detected increases.

In a preferable embodiment of the prediction method of the present invention, as a method for detecting the expression of a PLsis marker gene, quantitative PCR is used. As quantitative PCR, for example, competitive PCR, real-time PCR and the like can be mentioned. Since electrophoresis after amplification reaction is not necessary, real-time PCR is more superior in rapid performance.

In the case of competitive PCR, a known amount of a competitor nucleic acid is used, which can be amplified by said primer set, and after amplification, can be distinguished from an amplification product of a target nucleic acid (i.e., a transcription product of a PLsis marker gene), based on different amplification size, different migration pattern of restriction enzyme-treated fragment and the like in addition to the primer set described for the above-mentioned reagent of the present invention. Since amplification occurs competitively where a target nucleic acid and a competitor nucleic acid compete for a primer, the amount ratio of the amplification products reflects the amount ratio of the original templates. The competitor nucleic acid may be DNA or RNA. In the case of DNA, cDNA is synthesized from an RNA fraction prepared as mentioned above by reverse transcription reaction, and then PCR is performed in the coexistence of the reagent of the present invention and a competitor, and in the case of RNA, a competitor is added to an RNA fraction to allow reverse transcription reaction, and the reagent of the present invention is further added to perform PCR.

In real-time PCR, the amplification amount is monitored in real-time using a fluorescent reagent, and an apparatus integrally comprising a thermal cycler and a spectrofluoro-photometer is necessary. Such apparatus is commercially available. There are several methods depending on the fluorescent reagent to be used and, for example, intercalator method, TaqMan™ probe method, Molecular Beacon method and the like can be mentioned. In any case, cDNA is synthesized by reverse transcription reaction from an RNA fraction prepared as mentioned above, and the reagent of the present invention and a fluorescent reagent (probe) called intercalator, TaqMan™ probe or Molecular Beacon probe are added to PCR reaction system. Since intercalator binds to a synthesized double stranded DNA and emits fluorescence upon irradiation of excitation light, the amount of an amplification product can be monitored by measuring the intensity of fluorescence, based on which the amount of original template cDNA can be assumed. The TaqMan™ probe is an oligonucleotide capable of hybridizing to an amplification region of the target nucleic acid, which has both ends modified by a fluorescent substance and a quenching substance, respectively. It hybridizes to a target nucleic acid during annealing but is prohibited from emitting fluorescence by the presence of the quenching substance, and emits fluorescence when decomposed by the exonuclease activity of DNA polymerase during elongation, which releases the fluorescent substance. Accordingly, by measuring fluorescence intensity, the amount of the amplification product can be monitored, based on which the amount of original template cDNA can be assumed. The Molecular Beacon probe is an oligonucleotide capable of hybridizing to an amplification region of a target nucleic acid and having a hairpin type secondary structure, which has both ends modified by a fluorescent substance and a quenching substance, respectively. When it has a hairpin structure, it does not emit fluorescence due to the presence of a quenching substance, and emits fluorescence when the distance between the fluorescent substance and the quenching substance grows upon hybridization to the target nucleic acid during annealing. Therefore, the amount of the amplification product can be monitored by measuring the fluorescence intensity, based on which the amount of original template cDNA can be assumed.

Alternatively, expression of PLsis marker gene in a sample from a mammal can be determined by preparing a protein fraction from the sample and detecting a translation product (i.e., marker protein) of the marker gene in the fraction. Detection of a marker protein can be performed by an immunological measurement method (e.g., ELISA, FIA, RIA, Western blot etc.) using an antibody to each protein, and can also be performed by measuring the physiological activity of a protein showing measurable physiological activity of enzyme and the like, by a known method for each marker protein. Alternatively, a marker protein can be detected by mass spectrometry such as MALDI-TOFMS and the like.

The antibody to each marker protein can be obtained according to a general technique used for the production of a polyclonal antibody or monoclonal antibody, which uses, as a sensitizing antigen, a protein or partial peptide thereof, or a salt thereof having the same or substantially the same amino acid sequence as the amino acid sequence shown in SEQ ID NO: m (wherein m is an even number between 2 and 58, or an odd number between 65 and 99), or a protein or partial peptide thereof, or a salt thereof having the same or substantially the same partial amino acid sequence as the partial amino acid sequence encoded by each base sequence shown in SEQ ID NO: n (wherein n is an integer number between 59 and 63, 100 or 101).

In the determination method of the present invention, the standard according to which the presence or absence of a PLsis expression is judged is not particularly limited as long as the determination results based on the standard have sufficient reliability for use as a diagnostic system (compound evaluation system in clinical or preclinical tests) for the pathology. For example, (1) a method for determining PLsis positive when expression of all PLsis marker genes to be the detection target increases or decreases (where “expression substantially increases or decreases” means as defined above), and determining PLsis negative when expression of any of the PLsis marker genes does not substantially change (where “expression does not substantially change” means as defined above), (2) a method for determining PLsis negative when expression of all PLsis marker genes to be the detection target does not substantially change, and determining PLsis positive when expression of any of PLsis marker genes substantially increases or decreases, (3) a method for determining PLsis positive when expression of not less than a certain number (e.g., 2-(n−1) genes) out of PLsis marker genes in the number of n to be the detection target substantially increases or decreases and the like can be mentioned. However, the above-mentioned method (1) is defective in that the frequency of appearance of false-positive can be reduced, but the frequency of appearance of false-negative increases and a considerable number of PLsis expressions may be overlooked. On the other hand, according to the method of (2), the frequency of appearance of false-negative can be reduced, but the frequency of appearance of false-positive increases, which possibly eliminates potential compounds in, for example, clinical or preclinical tests, and the room of development of pharmaceutical products and the like may be narrowed.

As diagnostic criteria for avoiding the above-mentioned problems and determining PLsis expression with higher precision, for example, use of Phospholipidosis mRNA score (to be referred to as the “average variation rate” in the present specification) described in above Sawada, H. et al., Toxicol. Sci., 83: 282-92 (2005) can be mentioned. The “average variation rate” is defined as follows. That is, an expression amount is measured for each marker gene when mammal is and is not exposed to a test compound and upon exposure, when the expression amount increased, its magnification (e.g., 2 when increased two-fold) is taken as an expression variation rate (X) of each gene, and when the expression amount decreased, an inverse number of its magnification (e.g., 2 when decreased to ½) is taken as an expression variation rate (X) of each gene, and an average value of the expression variation rate of the total marker genes (n genes) is defined to be an average variation rate (following formula).

Average variation rate=m₁X₁+m₂X₂+ . . . +mₙXₙ(m₁+m₂+ . . . +mₙ=1)

wherein mᵢ (i=1−n) shows the weight of each gene. While the weight is not particularly limited, it is preferably mᵢ×n=0.2-5, for example, it is the same weight for all (mᵢ=1/n).

For example, a mammal is exposed to each of 2 or more (preferably 5 or more, more preferably 10 or more, more preferably 15 or more) known PLsis-inducing compounds and 2 or more (preferably 5 or more, more preferably 10 or more, more preferably 15 or more) known PLsis-noninducing compounds, in the sample from the mammal, expression variation of one or more PLsis marker genes selected (at least one of them is among the above-mentioned 54 marker genes of the present invention) is detected, and the average variation rate of expression of the marker gene and the presence or absence of actual PLsis expression are compared. The presence or absence of actual PLsis expression can be determined using vacuolated lymphocyte ratio of the peripheral blood from a mammal, or histopathological finding in one or more from various tissues (e.g., foam cell humectant in the lung and mesenteric lymph node, vacuolation of hepatocyte or bile duct epithelial cell in the liver, vacuolation of lymphocyte or foam cell humectant in the spleen, vacuolation of renal tubule epithelial cell in the kidney, vacuolation of cerebellar Purkinje cell in the brain) as an index.

As a result of comparison, an average variation rate affording correct determination of the presence or absence of the PLsis-inducibility of the above-mentioned known PLsis-inducing and non-inducing compounds with the probability of not less than about 70%, preferably not less than about 80%, more preferably not less than about 90%, particularly preferably not less than about 95%, is determined and used as a standard value. It is more preferable to study the validity of the standard value determined in this way by comparing average variation rate of PLsis marker gene expression and the presence or absence of actual PLsis expression in the same manner, using different known PLsis-inducing and noninducing compounds. When the average variation rate value affording accurate determination of the presence or absence of PLsis expression with higher probability by combining the determination results of the compounds studied anew is obtained, the standard value only needs to be amended.

Examples of other preferable diagnostic criteria applicable to the determination method of the present invention include, but are not limited to, those according to the diagnostic criteria of hepatotoxicity/nephrotoxicity-inducing potential by a compound as described in WO 02/10453 and WO 02/095000.

Abbreviations for bases, amino acids and the like used in the present specification are based on abbreviations specified by the IUPAC-IUB Commission on Biochemical Nomenclature or abbreviations in common use in relevant fields. Some examples are given below. When an enantiomer may be present in amino acid, it is of the L-configuration, unless otherwise stated.

DNA: Deoxyribonucleic acid
cDNA: Complementary deoxyribonucleic acid

### A: Adenine

### T: Thymine

### G: Guanine

### C: Cytosine

RNA: Ribonucleic acid
mRNA: Messenger ribonucleic acid
dATP: Deoxyadenosine triphosphate
dTTP: Deoxythymidine triphosphate
dGTP: Deoxyguanosine triphosphate
dCTP: Deoxycytidine triphosphate
ATP: Adenosine triphosphate
EDTA: Ethylenediaminetetraacetic acid
SDS: Sodium dodecyl sulfate

### Gly: Glycine

### Ala: Alanine

### Val: Valine

### Leu: Leucine

### Ile: Isoleucine

### Ser: Serine

### Thr: Threonine

### Cys: Cysteine

### Met: Methionine

Glu: Glutamic acid
Asp: Aspartic acid

### Lys: Lysine

### Arg: Arginine

### His: Histidine

### Phe: Phenylalanine

### Tyr: Tyrosine

### Trp: Tryptophan

### Pro: Proline

### Asn: Asparagine

### Gln: Glutamine

pGlu: Pyrroglutamic acid

### Sec: Selenocysteine

The present invention is explained in more detail in the following by referring to Examples, which are mere examples and do not limit the scope of the present invention in any way.

### Example 1

6 kinds of known PLsis-inducing compounds (amiodarone:1000 mg/kg/day, tamoxifen:1000 mg/kg/day, chloroquine:250 mg/kg/day, perhexiline: 600 mg/kg/day, fluoxetine:300 mg/kg/day and quinacrine:200 mg/kg/day) were administered by gavage to 5-week-old Crj:CD(SD)IGS rats (Charles River Japan Inc., produced in closed environment) at a dosing liquid volume of 10 mL/kg/day once daily for 3 days (4 days for the negative control (0.5 w/v % methylcellulose solution administration)).

After completion of the administration, in all animals, livers, kidneys, spleens, lungs and mesenteric lymph nodes were sampled, with additional organs and tissues sampled for respective test compounds, i.e., adrenal glands, pituitary glands and eyeballs for tamoxifen, brains and femoral muscles for quinacrine, brains and eyeballs for chloroquine, brains and skins for perhexiline, and brains for fluoxetine; these sampled tissues were examined for histopathological changes by conventional methods of optical microscopy and electron microscopy. In addition, a blood smear sample (Giemsa stain sample) was prepared, and the vacuolated lymphocyte ratio of the peripheral blood was measured with observation under a microscope.

The results for histopathological changes and vacuolated lymphocyte ratio in the PLsis-inducing compound administration group are shown in Table 1. In all histopathological tests in the compound administration group, changes considered to be attributable to PLsis were observed. Simultaneously, the vacuolated ratio of lymphocyte was found to be high in a vacuolated lymphocyte test of peripheral blood.

TABLE 1
Vacuolated lymphocyte ratio and histopathological finding
in PLsis-inducing compound administrated rat

| | | Vacuolated | Histopathological findings (PLsis) | | | | |
|---|---|---|---|---|---|---|---|
| | Dose | lymphocyte | | Lymph | | | |
| Compound | (mg/kg/day) | ratio (%) | Lung | node | Liver | Spleen | Others |
| Amiodarone | 1000 | 40↑ | + | + | − | + | − |
| Tamoxifen | 1000 | 40↑ | + | + | + | − | − |
| Chloroquine | 250 | 66↑ | − | + | − | + | − |
| Perhexiline | 600 | 52↑ | + | + | − | + | − |
| Fluoxetine | 300 | 31↑ | + | + | + | − | brain |
| Quinacrine | 200 | 32↑ | + | + | + | − | − |

↑: high value; +: PLsis lesion; −: no PLsis lesion

### Example 2

At 24 hr after completion of the oral administration for 3 consecutive days in Example 1, the blood was collected from the compound administration group and the control (non-administration) group (3 rats per group), which was pooled and subjected to GeneChip analysis.

After collection using PAXgene Blood RNA Tube (PreAnalytiX), the blood was stood for 2 hr or more, and cryopreserved at −80° C. Total RNA was extracted using a PAXgene Blood RNA Kit (PreAnalytiX). A sample for GeneChip analysis was prepared and analyzed according to the protocol provided by Affymetrix Inc. cDNA synthesis reaction was carried out using a SuperScript Choice system (Invitrogen), then biotin-labeled cRNA synthesis reaction was carried out using Enzo BioArray HighYield RNA Transcript Labeling Kit (Affymetrix), after which cRNA was fragmented and a labeled sample was prepared. The labeled sample was hybridized to Rat Expression Array 230A (RAE230A, Affymetrix), the array was stained, washed and scanned by GeneChip system (Affymetrix), and the following three image data were obtained from one sheet of array.

### Image Data

1. data of staining by a method omitting fluorescence amplification reaction by antibody
2. data of staining by the method described in a manual for once performing fluorescence amplification reaction by antibody
3. data of staining by a method for twice performing fluorescence amplification reaction by antibody

From the image data, measurement value (Signal), numerical data such as Signal Log Ratio and the like and determination data such as Detection, Change and the like were obtained using MicroarraySuite5.0 (parameter: Default value). Fold Change was calculated from Signal Log Ratio of the image data 2 using the conversion equation of Fold Change=2^{Signal Log Ratio}.

Then, using a DNA microarray data analysis soft, GeneSpring (Silicon Genetics), the probe set* that showed variation by compound addition was extracted according to the following criteria, and used as a variation probe set. (*In GeneChip analysis using RAE230A, 11 sites per one measurement target sequence are measured, and the measurement results at 11 sites are combined to give one numerical value and determination data. RAE230A contains 15924 combinations of 11 site measurement sets, and each measurement set is referred to as a probe set.)

### Extraction Criteria of Variation Probe Set

A probe set wherein two or more of the above-mentioned three image data satisfy the following three conditions was taken as a variation probe set.

1. A probe set judged as Increase or Decrease by MicroarraySuite5.0
2. A probe set whose higher measurement value (Signal) was judged as Presence by MicroarraySuite5.0
3. A probe set wherein Signal Log Ratio of treatment group shows 0.6 or above or −0.6 or below, based on the control group

The annotation information of the gene name, function, sequence and the like of the probe set was obtained from HumanPSD (Incyte), NetAffx (database relating to target mounted on GeneChip, Affymetrix).

The probe sets with expression level variation were extracted and the number is summarized in Table 2.

TABLE 2
Number of expression variation probe sets in
rat administrated with PLsis-inducinq compound

| | Dose | Increased | Decreased |
|---|---|---|---|
| Compound | (mg/kg/day) | expression | expression |
| Amiodarone | 1000 | 613 | 282 |
| Tamoxifen | 1000 | 258 | 94 |
| Chloroquine | 250 | 382 | 318 |
| Perhexiline | 600 | 310 | 179 |
| Fluoxetine | 300 | 372 | 391 |
| Quinacrine | 200 | 240 | 392 |

In all 6 compounds, expression variation was observed as follows:

increase: 9 probe sets (8 genes) (Table 3),
decrease: 27 probe sets (26 genes) (Table 4).

TABLE 3
Probe set with increase of expression in all of six PLsis-inducing compound 3-day administration groups

| | Affymetrix | Accession | | Gene | | Corresponding | | |
|---|---|---|---|---|---|---|---|---|
| Category | No. | No. | Gene name | symbol | Function | human gene | a | b |
| transport | 1386902_at | NM_031355 | voltage- | Vdac3 | protein at opening of | NM_005662 | 0 | |
| system | | | dependent anion | | voltage-dependent ion | | | |
| | | | channel 3 | | channel of mitochondrial | | | |
| | | | | | outer membrane that | | | |
| | | | | | possibly functions for | | | |
| | | | | | transport of adenine | | | |
| | | | | | nucleotides | | | |
| transport | 1367945_at | NM_053359 | ATX1 | Atox1 | copper binding protein that | NM_004045 | 0 | |
| system/ | | | (antioxidant | | mediates intracellular | | | |
| antioxidant | | | protein 1) | | copper transport and | | | |
| action | | | homolog 1 | | homeostasis | | | |
| | | | (yeast) | | | | | |
| antioxidant | 1370172_at | NM_017051 | superoxide | Sod2 | mitochondrial enzyme that | NM_000636 | 0 | |
| action | | | dismutase 2, | | converts superoxide to | | | |
| | | | mitochondrial | | hydrogen peroxide | | | |
| cell | 1367693_at | NM_013052 | tyrosine 3- | Ywhah | apoptosis inhibitor and | NM_003405 | 0 | |
| cycle · | | | monooxygenase/ | | activator of tyrosine | | | |
| growth · | | | tryptophan 5- | | hydroxylase and tryptophan | | | |
| death | | | monooxygenase | | hydroxylase | | | |
| | | | activation | | | | | |
| | | | protein, eta | | | | | |
| | | | polypeptide | | | | | |
| protease | 1367998_at | NM_053372 | secretory | Slpi | inhibitor of protease such | NM_003064 | 6 | |
| inhibitor | | | leukocyte | | as trypsin, cathepsin G | | | |
| | | | protease | | and neutrophil elastase | | | |
| | | | inhibitor | | and the like | | | |
| immune | 1367850_at | NM_053843 | Fc receptor, | Fcgr3 | binds with immunoglobulin | NM_000569, | 3 | |
| response | | | IgG, low | | and initiates various | NM_000570 | | |
| | | | affinity III | | immune responses | | | |
| | 1398246_s_at | NM_{——}053843 | Fc receptor, | Fcgr3 | binds with immunoglobulin | NM_000569 | 4 | |
| | | | IgG, low | | and initiates various | NM_000570 | | |
| | | | affinity III | | immune responses | | | |
| | 1389123_at | NM_001004202 | chemokine (C-C | cc16 | CC chemokine that promotes | none | 6 | |
| | | | motif) ligand 6 | | migration and infiltration | | | |
| | | | | | of inflammatory cell | | | |
| | 1371440_at | NM_012512 | Beta-2 | B2m | component of MHC class I | AK026463, | 1 | AW916647 |
| | | | microglobulin | | antigen that acts on | NM_004048 | | |
| | | | | | transepithelial transport | | | |
| | | | | | of IgG | | | |

a) number of compounds showing increase in the expression even in 7-day administration group
b) Accession No. of EST containing partial sequence

TABLE 4
Probe set with decrease of expression in all of six PLsis-inducing compound 3-day
administration groups

| | | | | | | Corresponding | | |
|---|---|---|---|---|---|---|---|---|
| | | | | | | human | | |
| Category | Affymetrix No. | Accession No. | Gene name | Gene symbol | Function | gene | a | b |
| Transport | 1367960_at | NM_019186 | ADP-ribosylation- | Ar14 | vesicular transport | NM_005738, | 0 | |
| system | | | like 4 | | and protein | NM_212460 | | |
| | | | | | secretion | | | |
| | | | | | associated GTP | | | |
| | | | | | binding protein | | | |
| | 1371469_at | NM_024139 | calcium binding | Chp | membrane transport | NM_007236 | 4 | |
| | | | protein p22 | | associated Ca2+ | | | |
| | | | | | binding protein | | | |
| | 1387388_at | NM_024139 | calcium binding | Chp | membrane transport | NM_007236 | 5 | |
| | | | protein p22 | | associated Ca2+ | | | |
| | | | | | binding protein | | | |
| | 1371908_at | XM_215858 | Similar to NTF2- | — | functions in | NM_013248 | 4 | AA891920 |
| | | | related export | | nuclear transport | | | |
| | | | protein NXT1 | | pathway | | | |
| | | | (LOC296219), mRNA | | | | | |
| | 1387651_at | NM_012778 | aquaporin 1 | Aqp1 | water channel | NM_000385, | 4 | L07268 |
| | | | (channel-forming | | | NM_198098 | | |
| | | | integral protein, | | | | | |
| | | | 28 kDa) | | | | | |
| | 1388480_at | XM_213793 | Similar to | — | intermembrane | NM_016433 | 2 | AI412863 |
| | | | Glycolipid | | transport of | | | |
| | | | transfer protein | | glycosphingolipid, | | | |
| | | | (LOC288707), mRNA | | glycoglycerolipid | | | |
| Anti- | 1367613_at | NM_057114 | peroxiredoxin 1 | Prdx1 | antioxidant having | NM_002574, | 2 | |
| oxidant | | | | | peroxydase | NM_181696, | | |
| action | | | | | activity induced by | NM_181697 | | |
| | | | | | oxidative stress | | | |
| | 1398839_at | NM_053800 | thioredoxin | Txn | involved in | NM_003329 | 3 | |
| | | | | | response to UV and | | | |
| | | | | | oxidative stress, | | | |
| | | | | | decreases reactive | | | |
| | | | | | oxygen intermediate | | | |
| cell | 1367780_at | NM_022391 | pituitary tumor- | Pttg1 | premalignant gene, | NM_004219 | 4 | |
| cycle· | | | transforming 1 | | downregulated in | | | |
| growth · | | | | | response to serum | | | |
| death | | | | | starvation, | | | |
| | | | | | promotes cell | | | |
| | | | | | growth and | | | |
| | | | | | angiogenesis | | | |
| | 1375428_at | XM_213921 | Similar to | — | involved in | NM_003851 | 5 | BE099979 |
| | | | cellular repressor | | transcriptional | | | |
| | | | of E1A-stimulated | | regulation in cell | | | |
| | | | genes CREG | | growth or | | | |
| | | | (LOC289185), mRNA | | differentiation | | | |
| | 1381968_at | AI029175 | Similar to | — | involved in | NM_003851 | 5 | |
| | | | cellular repressor | | transcriptional | | | |
| | | | of E1A-stimulated | | regulation in cell | | | |
| | | | genes CREG | | growth or | | | |
| | | | (LOC289185), mRNA | | differentiation | | | |
| | 1375896_at | BM392055 | Similar to | — | antiapoptopic | NM_018571 | 3 | |
| | | | amyotrophic | | protein | | | |
| | | | lateral sclerosis | | potentiating | | | |
| | | | 2 (juvenile) | | activation of JNK1 | | | |
| | | | chromosome region, | | | | | |
| | | | candidate 2 | | | | | |
| | 1388286_a_at | L38482 | Similar to cell | — | ubiquitin | NM_004359 | 1 | |
| | | | division cycle 34; | | conjugating enzyme | | | |
| | | | ubiquitin- | | that regulates | | | |
| | | | conjugating enzyme | | transfer from G1 | | | |
| | | | E2-32 KDA | | phase to S phase | | | |
| | | | complementing; | | and chromosome | | | |
| | | | ubiquitin carrier | | alignment | | | |
| | | | protein; | | | | | |
| | | | ubiquitin-protein | | | | | |
| | | | ligase | | | | | |
| | 1388387_at | NM_001007742 | ubiquitin | Ubadc1 | putative | NM_016172 | 3 | BI275880 |
| | | | associated domain | | intracellular | | | |
| | | | containing 1 | | signal molecule | | | |
| | | | | | capable of | | | |
| | | | | | regulating growth | | | |
| | | | | | of endothelial cell | | | |
| RNA | 1373200_at | AI600237 | Similar to eukaryotic | — | translation | NM_004280 | 3 | |
| transcript | | | translation | | elongation factor | | | |
| | | | elongation factor 1 | | | | | |
| | | | epsilon 1 | | | | | |
| | | | (LOC291057), mRNA | | | | | |
| | 1389680_at | XM_226624 | Similar to RNA | — | member of RNA | NM_012081 | 5 | BI291626 |
| | | | polymerase II | | polymerase II | | | |
| | | | elongation factor | | elongation factor | | | |
| | | | ELL2 (LOC309918), | | ELL2 family | | | |
| | | | mRNA | | | | | |
| | 1388914_at | XM_214451 | Similar to germ cell | — | RNA binding | NM_015982 | 4 | BI288013 |
| | | | specific Y-box | | protein, involved | | | |
| | | | binding protein | | in translational | | | |
| | | | | | repression of mRNA | | | |
| | | | | | in germ cell | | | |
| protease | 1367646_at | NM_022597 | cathepsin B | Ctsb | cysteine protease | NM_001908, | 1 | |
| | | | | | belonging to | NM_147780, | | |
| | | | | | papain family | NM_147781, | | |
| | | | | | derived from | NM_147782, | | |
| | | | | | lysosome | NM_147783 | | |
| | 1370460_at | NM_145184 | ubiquitin specific | Usp15 | enzyme belonging | NM_006313 | 3 | |
| | | | protease 15 | | to ubiquitin | | | |
| | | | | | specific cysteine | | | |
| | | | | | protease family | | | |
| cytoskeleton | 1368541_at | NM_053719 | embigin | Emb | transmembrane | NM_198449 | 3 | |
| | | | | | protein, cell | | | |
| | | | | | adhesion molecule | | | |
| | 1389398_at | AI172141 | Similar to Ankyrin 1 | — | cytoskeletal | NM_000037, | 1 | |
| | | | | | anchoring protein | NM_020475, | | |
| | | | | | that adheres | NM_020476, | | |
| | | | | | cytoskeletal | NM_020477 | | |
| | | | | | element to plasma | | | |
| | | | | | membrane | | | |
| Others | 1367632_at | NM_017073 | glutamine synthetase 1 | Glu1 | glutamine | NM_002065 | 0 | |
| | | | | | synthetase | | | |
| | 1367650_at | NM_053582 | lipocalin 7 | Lcn7 | putative | NM_022164 | 5 | |
| | | | | | extracellular | | | |
| | | | | | matrix protein, | | | |
| | | | | | cathepsin B | | | |
| | | | | | associated protein | | | |
| | | | | | inactive as | | | |
| | | | | | catalyst | | | |
| | 1370913_at | NM_138881 | Best5 protein | Best5 | responses to | NM_080657 | 3 | |
| | | | | | interferon, | | | |
| | | | | | similar to human | | | |
| | | | | | viperin | | | |
| | 1372191_at | XM_340803 | Similar to Sin3A | — | similar to | NM_024632 | 3 | BI303656 |
| | | | associated protein | | transcription | | | |
| | | | p30-like | | corepressor Sin3A | | | |
| | | | | | associated protein | | | |
| | | | | | (SAP30) related to | | | |
| | | | | | histone | | | |
| | | | | | deacetylase | | | |
| Unknown | 1375526_at | XM_213333 | Similar to | — | | NM_152766 | 6 | AI171327 |
| | | | hypothetical protein | | | | | |
| | | | MGC40107 (LOC287442), | | | | | |
| | | | mRNA | | | | | |
| | 1388493_at | AI600030 | — | — | | none | 3 | |

a) number of compounds showing decrease in the expression even in 7-day administration group
b) other Accession No. of the same gene or Accession No. of EST containing partial sequence

### Example 3

6 kinds of known PLsis-inducing compounds (amiodarone:300 mg/kg/day, tamoxifen:100 mg/kg/day, chloroquine:75 mg/kg/day, perhexiline: 200 mg/kg/day, imipramine:100 mg/kg/day and quinacrine:60 mg/kg/day, negative control:0.5 w/v % methylcellulose solution) were administered by gavage to 5-week-old Crj:CD(SD)IGS rats (Charles River Japan Inc., produced in closed environment) at a dosing liquid volume of 10 mL/kg/day once daily for 7 days.

After completion of the administration, in all animals, livers, kidneys, spleens, lungs and mesenteric lymph nodes were sampled, these sampled tissues were examined for histopathological changes by conventional methods of optical microscopy and electron microscopy. In addition, a blood smear sample (Giemsa stain sample) was prepared, and the vacuolated lymphocyte ratio of the peripheral blood was measured with observation under a microscope.

The results for histopathological changes and vacuolated lymphocyte ratio in the PLsis-inducing compound administration group are shown in Table 5. In all the histopathological tests in the compound administration group, changes considered to be attributable to Plsis were observed. Simultaneously, the vacuolated ratio of lymphocyte was found to be high in a vacuolated lymphocyte test of peripheral blood.

TABLE 5
Vacuolated lymphocyte ratio and histopathological finding
in PLsis-inducing compound administrated rat

| | | Vacuolated | Histopathological findings (PLsis) | | | | |
|---|---|---|---|---|---|---|---|
| | Dose | lymphocyte | | | | | Lymph |
| Compound | (mg/kg/day) | ratio (%) | Lung | Liver | Spleen | Kidney | node |
| Negative | 0 | 0.0 | − | − | − | − | − |
| control | | | | | | | |
| Amiodarone | 300 | 31.5↑ | ++ | − | − | − | ++ |
| Imipramine | 100 | 12.3↑ | + | ++ | − | − | − |
| Tamoxifen | 100 | 10.3↑ | + | − | − | − | ++ |
| Quinacrine | 60 | 16.0↑ | + | ++ | + | + | − |
| Chloroquine | 75 | 16.3↑ | − | + | + | − | − |
| Perhexiline | 200 | 12.5↑ | + | − | − | − | + |

↑: high level; ++: severe PLsis lesion; +: mild PLsis lesion; −: no PLsis lesion

### Example 4

At 24 hr after completion of the oral administration for 7 consecutive days in Example 3, the blood was collected from the compound administration group and the control (non-administration) group (4 rats per group), which was pooled and subjected to GeneChip analysis in the same manner as in Example 2.

The probe sets with expression level variation were extracted and the number is summarized in Table 6.

TABLE 6
Number of expression variation probe sets in
rat administrated with PLsis-inducing compound

| | Dose | Increased | Decreased |
|---|---|---|---|
| compound | (mg/kg/day) | expression | expression |
| Amiodarone | 300 | 515 | 457 |
| Imipramine | 100 | 123 | 320 |
| Tamoxifen | 100 | 546 | 368 |
| Quinacrine | 60 | 258 | 103 |
| Chloroquine | 75 | 310 | 179 |
| Perhexiline | 200 | 49 | 83 |

In all 6 compounds, expression variation was observed as follows:

increase: 14 probe sets (14 genes) (Table 7),
decrease: 9 probe sets (9 genes) (Table 8). Of these, expression variation was observed in all the tested 6 compounds (5 compounds were duplicated) even after 3 days of administration in chemokine (C-C motif) ligand 6 (NM_{—}001004202), secretory leukocyte peptidase inhibitor (NM_{—}053372) and similar to hypothetical protein MGC40107 (XM_{—}213333).

TABLE 7
Probe set with increase of expression in all of six PLsis-inducing compound 7-day administration groups

| | Affymetrix | Accession | | Gene | | Corresponding | |
|---|---|---|---|---|---|---|---|
| Category | No. | No. | Gene name | symbol | Function | human gene | a |
| Lipid- | 1367614_at | NM_012904 | annexin A1 | Anxa1 | calcium-dependent | NM_000700 | 3 |
| associated | | | | | phospholipid binding protein | | |
| | | | | | that inhibits phospholipase | | |
| | | | | | A2 | | |
| | 1370180_at | NM_053598 | nudix (nucleoside | Nudt4 | diphosphoinositol | NM_019094, | 5 |
| | | | diphosphate | | polyphosphate | NM_199040 | |
| | | | linked moiety X)- | | phosphohydrolase possibly | | |
| | | | type motif 4 | | involved in metabolism of | | |
| | | | | | inositol phosphate | | |
| | 1373877_at | NM_053554 | Phosphatidyl | Picalm | bound with clathrin heavy | NM_001008660, | 3 |
| | | | inositol binding | | chain, acts on endocytosis | NM_007166 | |
| | | | clathrin assembly | | | | |
| | | | protein | | | | |
| | 1368494_at | NM_053822 | S100 calcium | S100a8 | member of S100 family, forms | NM_002964 | 4 |
| | | | binding protein | | a complex with S100A9 and | | |
| | | | A8 (calgranulin | | mediates arachidonic acid | | |
| | | | A) | | secretion and leukocyte | | |
| | | | | | supplementation to a site of | | |
| | | | | | inflammation | | |
| | 1387125_at | NM_053587 | S100 calcium | S100a9 | member of S100 family, forms | NM_002965 | 3 |
| | | | binding protein | | a complex with S100A8 and | | |
| | | | A9 (calgranulin | | mediates arachidonic acid | | |
| | | | B) | | secretion and leukocyte | | |
| | | | | | supplementation to a site of | | |
| | | | | | inflammation | | |
| | 1371615_at | NM_001012345 | Diacylglycerol 0- | Dgat2 | member of diacylglycerol | NM_032564 | 0 |
| | | (XM_341887) | acyltransferase | | acyltransferase family, | | |
| | | | homolog 2 (mouse) | | catalyzes triacylglycerol | | |
| | | | | | biosynthesis | | |
| Immune | 1373661_a_at | NM_022205 | Chemokine (C-X-C | Cxcr4 | GPCR, which is bound with | NM_001008540, | 3 |
| reaction | | | motif) receptor 4 | | CXC chemokine binding | NM_003467 | |
| | 1389123_at | NM_001004202 | chemokine (C-C | Cc16 | CC chemokine that promotes | | 6 |
| | | | motif) ligand 6 | | migration and humectant of | | |
| | | | | | inflammatory cell | | |
| | 1398256_at | NM_031512 | interleukin 1 | 111b | cytokine that regulates | NM_000576 | 4 |
| | | | beta | | defense reaction and | | |
| | | | | | inflammation reaction | | |
| | 1367998_at | NM_053372 | secretory | Slpi | protease inhibitor such as | NM_003064 | 6 |
| | | | leukocyte | | trypsin, cathepsin G and | | |
| | | | peptidase | | neutrophil elastase and the | | |
| | | | inhibitor | | like | | |
| Others | 1372776_at | XM_223508 | F-box and | Fbx15 | putative subunit of SCF | NM_012161, | 4 |
| | | | leucine-rich | | ubiquitin ligase involved in | NM_033535 | |
| | | | repeat protein 5 | | proteolysis | | |
| | | | (predicted) | | | | |
| | 1387422_at | NM_053373 | peptidoglycan | Pglyrp1 | Bound with peptidoglycan and | NM_005091 | 2 |
| | | | recognition | | gram-positive bacterium, | | |
| | | | protein 1 | | involved in congenital | | |
| | | | | | immunity | | |
| | 1399158_a_at | NM_012992 | nucleophosmin 1 | Npm1 | nucleic acid binding | NM_002520, | 2 |
| | | | | | ribonuclease, molecular | NM_199185 | |
| | | | | | chaperon, possibly involved | | |
| | | | | | in ribosome assembly | | |
| Unknown | 1374626_at | NM_001009717 | Similar to | | serum protein containing | NM_052972 | 5 |
| function | | | leucine-rich | | plural leucine-rich repeats | | |
| | | | alpha-2- | | | | |
| | | | glycoprotein | | | | |

a) number of compounds showing increase in the expression even in 3-day administration group

TABLE 8
Probe set with decrease of expression in all of six PLsis-inducing compound 7-day administration groups

| | Affymetrix | Accession | | Gene | | Corresponding | |
|---|---|---|---|---|---|---|---|
| Category | No. | No. | Gene name | symbol | Function | human gene | a |
| Lipid- | 1387796_at | NM_031010 | arachidonate 12- | Alox15 | enzyme that converts | NM_001140 | 3 |
| associated | | | lipoxygenase | | arachidonic acid to 15- | | |
| | | | | | hydroperoxyeicosatetraenoic | | |
| | | | | | acid and lipoxin A4 | | |
| Immune | 1372138_at | XM_341053 | Cut-like 1 | Cutl1 | acts on cell cycle of | NM_001913, | 1 |
| reaction/cell | | | (Drosophila) | | mitosis, possibly | NM_181500, | |
| growth- | | | | | regulates immune response | NM_181552 | |
| associated | | | | | to apoptosis-inducing | | |
| | | | | | stimulation | | |
| Cell growth- | 1372685_at | XM_214152 | cyclin-dependent | | tyrosine-serine | NM_005192 | 3 |
| associated | | | kinase inhibitor | | phosphatase that inhibits | | |
| | | | 3 (predicted) | | progression of cell cycle | | |
| Intracellular | 1372931_at | XM_346274 | Similar to | | member of prenylated Rab | NM_007213 | 1 |
| transport- | | | DXlmx39e protein | | acceptor (PRA1) family, | | |
| associated | | | | | moderately similar to | | |
| | | | | | glutamic acid transporter | | |
| Others | 1372273_at | NM_001013233 | Similar to | | erythrocyte transmembrane | NM_002101, | 1 |
| | | (XM_344660) | glycophorin C | | sialoglycoprotein, forms a | NM_016815 | |
| | | | isoform 2 | | triple complex with Epb4. | | |
| | | | | | 1 and Mpp1, possibly | | |
| | | | | | involved in regulation of | | |
| | | | | | cell shape | | |
| | 1389858_at | BM389006 | similar to | | cytoplasmic enzyme that | NM_003258 | 5 |
| | | | thymidine kinase1 | | synthesizes thymidine acid | | |
| | | | | | for DNA synthesis | | |
| Unknown | 1373948_at | XM_340780 | similar to | | member of uncharacterized | NM_012075 | 1 |
| function | | | CGTHBA protein | | protein UPF0171 family | | |
| | | | (−14 gene | | | | |
| | | | protein) | | | | |
| | | | (predicted) | | | | |
| | 1375526_at | XM_213333 | similar to | | member of unknown function | NM_152766 | 6 |
| | | | hypothetical | | protein (DUF423) family, | | |
| | | | protein MGC40107 | | possible membrane protein | | |
| | 1371970_at | AA799328 | | | | | 1 |

a) number of compounds showing decrease in the expression even in 3-day administration group

### INDUSTRIAL APPLICABILITY

The method for determining PLsis of the present invention uses, as an index, expression variation of a PLsis marker gene in blood or lymphocyte. Therefore, PLsis expression can be determined noninvasively and with high precision, as compared to conventional in vivo toxicity, tests and the like. Accordingly, the method is useful for the toxicity evaluation of drug candidate compounds in the later stage of drug development, such as clinical or preclinical stages and the like. In addition, the method is also useful for the diagnosis of other drug-induced PLsis, and hereditary or nonhereditary diseases associated with PLsis or PLsis-like changes as pathology.

While some of the preferable embodiments of the present invention have been explained with emphasis as mentioned above, it will, however, be evident for those of ordinary skill in the art that changes may be made to the preferable embodiments and that the present invention can be practiced in a manner other than specifically described in the present specification. Therefore, the present invention includes all the modifications encompassed in the spirit and scope of the invention as set forth in the appended claims.

All the references cited herein, including patents, patent applications and publications, are hereby incorporated in their entireties by reference.

This application is based on patent application Nos. 2005-040698 filed on Feb. 17, 2005 and 2005-128412 filed on Apr. 26, 2005 in Japan, the contents of which are incorporated in full herein by this reference.

1. A reagent for determining phospholipidosis in a mammal, which comprises a nucleic acid capable of hybridizing to a nucleic acid having a base sequence shown by any of SEQ ID NO n (wherein n is an odd number between 1 and 57, an integer number between 59 and 63, an even number between 64 and 98, 100 or 101) under high stringent conditions and/or a nucleic acid capable of hybridizing to a nucleic acid having a base sequence complementary to the base sequence under high stringent conditions.

2. A kit for determining phospholipidosis in a mammal, which comprises two or more reagents containing a nucleic acid capable of hybridizing to a transcription product of a gene showing varying expression in correlation with expression of phospholipidosis under high stringent conditions and/or a nucleic acid capable of hybridizing to a nucleic acid having a base sequence complementary to the transcription product under high stringent conditions, wherein,
(a) at least one reagent is the reagent of claim 1, and
(b) when two or more reagents of claim 1 are contained, each reagent can detect expression of different genes.

3. A method for determining phospholipidosis in a mammal, which comprises detecting expression variation of one or more genes showing expression variation in correlation with phospholipidosis expression, in a sample from a mammal, wherein at least one gene has the same or substantially the same base sequence as the base sequence shown by any of SEQ ID NO n (wherein n is an odd number between 1 and 57, an integer number between 59 and 63, an even number between 64 and 98, 100 or 101).

4. The method of claim 3, wherein the mammal is administered with a compound or exposed to the compound, and phospholipidosis is caused by the compound.

5. The method of claim 3, wherein the mammal is human.

6. The method of claim 3, wherein the mammal is rat, mouse, dog or monkey.

7. The method of claim 3, wherein the sample is blood or lymphocyte.


SEQUENCE LISTING (101 sequences)

SEQ ID NO: 1 - DNA, 1380 bp, Rattus norvegicus
  CDS (90)..(938)
aggtcgcgcaggcgcagacggcgttggttcgagaagaccttcagcgttgccttggtggagcggtacgggcccaccctcgggttgtagatatgtgtagcacaccaacttactgcgacctaggaaaggctgccaaggatgtctttaacaaagggtatgggtttggcatggtcaaaatagatctgaaaaccaagtcttgtagtggagtggaattttctacttctggtcatgcttatactgatacagggaaagcatcaggcaacctagagaccaaatataaggtctgtaactacgggctcatcttcacccaaaagtggaatacagacaatactcttgggacagaaatctcttgggagaataagttggctgaagggttgaaactgacggttgataccatatttgtaccaaacacagggaagaagagtgggaaattaaaggcctcctatagacgggattgttttagtgtgggcagtaaggttgacatagatttttctggaccgaccatctatggctgggccgtgttggcctttgaaggttggcttgctggctaccagatgagttttgacacagccaaatccaaactgtgtcagaataattttgctcttggttacaaggctgaagacttccaactgcatactcatgtaaacgatggcactgaatttggaggctccatctaccagagagttaatgagaagatcgaaacatcaataaacctggcatggacagctggcagcaacaacactcgttttggcatcgctgctaagtataggctggattgtagaacttctctgtctgccaaagtaaacaatgccagtttaattggactgggttatacgcagagtctccgaccgggagtcaaactgaccctgtcagctttagtggatggaaagaacttcaatgcaggaggccacaaggttggcttgggatttgaactggaagcttaatggggttttgagtagagtatcaattgtccctggaaatgaagagaaatgacaccactatgttttggccttaaaattctgtgaaatttcaaaagtgaactttttattcttccaaagaattgtaattcttcccacaccgaagtctagagattacagatccatccagtgggaggtccttgaaggcaatgcctggaaattgtcatgtttgtgccacatttcagttgagttctgcagagttaattttaaatatgttcctcagcaacaacgtagtgtcacgttaaaggaagtgatctgcccggtctgtacatcgtgtcctgcatgtcccaactccactttccatgaccttttgttacatcagtctctgctctagtgagacctttggtttgcatcagagtaaaataaacccatcacatttggaacataaaaaaaaaaaaaaaaaaaa

SEQ ID NO: 2 - protein, 283 aa, Rattus norvegicus
MCSTPTYCDLGKAAKDVFNKGYGFGMVKIDLKTKSCSGVEFSTSGHAYTDTGKASGNLETKYKVCNYGLIFTQKWNTDNTLGTEISWENKLAEGLKLTVDTIFVPNTGKKSGKLKASYRRDCFSVGSKVDIDFSGPTIYGWAVLAFEGWLAGYQMSFDTAKSKLCQNNFALGYKAEDFQLHTHVNDGTEFGGSIYQRVNEKIETSINLAWTAGSNNTRFGIAAKYRLDCRTSLSAKVNNASLIGLGYTQSLRPGVKLTLSALVDGKNFNAGGHKVGLGFELEA

SEQ ID NO: 3 - DNA, 503 bp, Rattus norvegicus
  CDS (114)..(317)
ctcgtgccgaatcggcacgagcggcacgagtggtgttcgggagaggcgttctagtcctcgccgccgcctgtgccgccgcctgtgccgcctgtgccgcctgtgccgcgtcagtcatgccgaagcacgagttctctgtggacatgacctgtggaggctgtgcggaagccgtctcccgagtcctcaacaagctgggaggagtggagttcaacattgacttgcccaacaagaaggtctgcatcgagtctgagcacagctcagacatcctgctggcaactctcaacaaaacaggaaaagcggtctcctaccttggccccaagtagccaggacctgggcgagtccttccggatagaaactgaagaggcagactgttgatctggtctcccccgctgatctggaacaccaactgctcagtccagctccgccatggaattccagcccagacaggccttccctgctggctccctgccagcttccttgcaattaaagtcaagctgcatttgttg

SEQ ID NO: 4 - protein, 68 aa, Rattus norvegicus
MPKHEFSVDMTCGGCAEAVSRVLNKLGGVEFNIDLPNKKVCIESEHSSDILLATLNKTGKAVSYLGPK

SEQ ID NO: 5 - DNA, 2090 bp, Rattus norvegicus
  CDS (84)..(749)
cgggcggacgccgcagagcagacgcgcggctgctagcgaacggccgtgttctgaggagagcagcggtcgtgggcgcctcagcaatgttgtgtcgggcggcgtgcagcgcgggcagaagactgggccccgcggccagtaccgcgggctcccggcacaagcacagcctccctgacctgccttacgactatggcgcgctggagccgcacattaacgcgcagatcatgcagctgcaccacagcaagcaccacgcgacctacgtgaacaatctgaacgtcaccgaggagaagtaccacgaggcgctggccaagggagatgttacaactcaggttgctcttcagcctgcactgaagttcaatggcgggggccatatcaatcacagcattttctggacaaacctgagccctaagggtggtggagaacccaaaggagagttgctggaggctatcaagcgtgactttgggtcttttgagaagtttaaggagaaactgacagctgtgtctgtgggagtccaaggttcaggctggggctggcttggcttcaataaggagcaaggtcgcttacagattgccgcctgctctaatcaggacccactgcaaggaaccacaggccttattccactgctggggattgatgtgtgggagcacgcttactatcttcagtataaaaacgtcagacctgactatctgaaagccatttggaatgtaatcaactgggagaatgttagccaaagatacatagtttgcaagaagtgaagcccttccgccaggctgtgtgtcaggcccgtggtgggtgttttgtagtagtgtagagcattgcagcactgtggctgagctgttgtaatcttcattgatgcctatccacatatgtgtaagcatacagttatgataatttcttaattaaatgtattgttaggcaactgtttgagaacagtacatacttggtgtgagctgctcttgattgaacattttcattagaggcttgaattgcttggacgctgtcactgtcatcataaggccatcaaagatattccatctctgtgttggggcctgtggggaggctgtaatcctgttctactgcagttaggaaaaaaaatgagttacccccccccccagaattgttgaataataaaatagagaactgaatagttctcttttctgttaaaaattgctatttttcataagtaatcctttgtttagcggatatcacctagtggtctttatttatggccacagtttcatagaaacatcattttttcacttgaaacgtgtaactaggctaaggatggatggagtggtagagcctttgcctgtcttatgtgaggccctgggctctacctcactactgaacaaatcaacagacccaagctaggctcctgactgacaactgttaattcggagaggagtgacattgtgcctctgggtttttttataggctgagatgcaaaaactgttaccttgtctattaaaaccgactgtgtattgtatgaaagtgctcaagatggacaaagtatgcagggcctgtcccatgatgtcaacatggggactaggccacagggcattcagggaagccattcagcaccattaattgtgtatctcaggagaactggaccctgggtgaggtggcacctttttcagtagaggcaaacgggtttctgagtgaggtcagtagggtgtcttgagaacatcagttttcagagacatgtattttcattttttctgcagtctcaagaacttatactcacaagagggcctttgtattgttattcaatagaaggacaccctttccctgacaaggtacacagtgtttctgattgtattcctgtgcagcatttgggtgtagagctgtgcactgttgaaatgcctttgtgaatttgccttttcacatttcgtctcaccatcttccttgttttctttactgactatgtaatgttttatcagttggatattactgaatagtttgtttgtgtttaattgccttttattaccaaaaaggctgtggtcagcaaccccaggcatgcacattacttcttaatggagtattttttaaaggtttattttaattttaaaaaaaaaaaaaaaaaaaaaaaaaaa

SEQ ID NO: 6 - protein, 222 aa, Rattus norvegicus
MLCRAACSAGRRLGPAASTAGSRHKHSLPDLPYDYGALEPHINAQIMQLHHSKHHATYVNNLNVTEEKYHEALAKGDVTTQVALQPALKFNGGGHINHSIFWTNLSPKGGGEPKGELLEAIKRDFGSFEKFKEKLTAVSVGVQGSGWGWLGFNKEQGRLQIAACSNQDPLQGTTGLIPLLGIDVWEHAYYLQYKNVRPDYLKAIWNVINWENVSQRYIVCKK

SEQ ID NO: 7 - DNA, 1689 bp, Rattus norvegicus
  CDS (176)..(913)
tgcagccagctagcgagaaggcgcgagcggcggcgcagccagcagcctcccgccagccggcgagccagtgcgcgtgcgcggcggcggcctcggcggcgaccgggaagcggacgggcgggcgaggcgagcgaggcaggcggtgcgggcgtgcgaggcgaggccgatcgcgagcgacatgggggaccgagagcagctgctgcagcgggcgcgactggcggagcaggcggagcgctacgacgacatggcctccgccatgaaggcggtgacagagctgaatgaacctctatctaatgaagatagaaatctcctctctgtggcctacaagaatgtagttggtgccaggcgatcttcttggagggttattagtagcattgagcagaaaaccatggcagatgggaatgagaagaagctggagaaagtcaaagcctatcgggagaagattgagaaggagctggagacagtttgcaatgatgtcttggctctgctcgacaagttccttatcaagaactgcaatgattttcagtacgagagcaaggtgttctacctgaaaatgaagggcgattactaccgctacctggcagaggtggcttctggggagaagaaaaacagtgtggttgaagcttctgaggcagcgtataaggaagccttcgaaatcagcaaagagcacatgcagccaacacaccccatccggcttggcctggccctcaatttttctgtgttctactatgagatccagaatgcaccagagcaggcctgcctcttagccaaacaagccttcgatgatgctatagctgagctggacacattaaacgaggattcctataaggactccactctcatcatgcagttgctgcgagacaacctcaccctctggacgagcgaccagcaggatgaagaagccggagaaggcaactgaagacccatcaggtccctggcccttcctttacccaccacccccattatcactgattcttccttgccacaatcactatatctagtgctaaacctatctgtattggcagcacagctattcagatctgccctcctgtcccttggaagcagtttcagataaaccttcatgggcatttgctggactgatggttgctttgagccacagagcgctccctttttgaattgtgcagagaagtgtgttctgaacgaggcattttattatgtctgttgatctgtagcaaatccatgtgatggtaattgagtgtagaaaggagaattagccaacacaggctatggctgctatttaaaacaagctgatagtgtgttgttaagcagtacatctcgtgcatgcaaaaatgaatttgaccctctcaccccttctttcagctaatggaaactgacacacgacaacttgttccttcaccatcagctttataaactgtttctcgtgagctttcaggcccctgctgtgcctctttaaattatgatgtgcgcacaccttcttttcaatgcaatgcatcagaggtttttgatatgtgtaacttttttttttggttgtgattaagaatcatggatttattttttgtaactctttggctattgttcttgtgtaccctgacagcatcatgtgtgtcaacctgtgtcaatcatgatgggtggttatgaaatgccagattgctaaaataaatgttttggacttaaaaagagtaaataaatgctgctttggggatattaaaa

SEQ ID NO: 8 - protein, 246 aa, Rattus norvegicus
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLALLDKFLIKNCNDFQYESKVFYLKMKGDYYRYLAEVASGEKKNSVVEASEAAYKEAFEISKEHMQPTHPIRLGLALNFSVFYYEIQNAPEQACLLAKQAFDDAIAELDTLNEDSYKDSTLIMQLLRDNLTLWTSDQQDEEAGEGN

SEQ ID NO: 9 - DNA, 667 bp, Rattus norvegicus
  CDS (13)..(402)
cctgccttcaccatgaagtccagcggcctgttccctctcatggtgctccttgctctgggtgtcctggcaccctggagtgtggaaggaggcaaaaatgatgctatcaaaatcggagcctgccctgctagaaagcctgcccagtgccttaaacgtgagaaaccagagtgcagtactgactggggatgcccaggaaagcagagatgctgccaagatacctgtggtttcaaatgcctgaatcctgttcccattcgtggaccagtgaagaagcctgggaggtgcctcaaatttcaaggaaaatgtctgatgcttaaccctcccaataagtgccagaatgacggccagtgtgatggcaaatacaaatgttgtgagggtatgtgtgggaaagtctgccttcccccagtgtaagtctgattcctgacatttggaaatggctctggacccgtgctctatatgttctggaaactacttccctgctttcaggtttcccggttccgggttccggtgctctggggtgcccaagctttggacatttgaataatgtttatttctgccgccaacatatctttttgtcccatgtcagcattcatcaagcccctaaggaaatgctgttggagacaaaataaataaaggcaatcatttctctaagcacaaaaaaaaaaaaaaaaaa

SEQ ID NO: 10 - protein, 130 aa, Rattus norvegicus
MKSSGLFPLMVLLALGVLAPWSVEGGKNDAIKIGACPARKPAQCLKREKPECSTDWGCPGKQRCCQDTCGFKCLNPVPIRGPVKKPGRCLKFQGKCLMLNPPNKCQNDGQCDGKYKCCEGMCGKVCLPPV

SEQ ID NO: 11 - DNA, 1424 bp, Rattus norvegicus
  CDS (237)..(1037)
cgcagcccagacaatgcttatccggaccggactccagcccacttctgccacctgaagggctccggttcgtgtgcaccgggaggcaccagagtctaagggcatccgggaagggactgttcggccggtccccgggcagccgctgggcgagaggaggaggctgcgccaaacagacagcaagcaagccttcgccagcagttcatctcctagacctcatcagactctgacccagttcttgaatgactttggagacccagatgtttcagaatgcacattctggaagccaatggctactcccaccactgacaatgttgctgctgtttgcttttgcagacaggcagacgggagatcttctgaaggctgtggtgaaacgtgatcccccatggatccaggtgctcaaggacgacactgtgacgctgacgtgcgaagggacccacaatcctggaaactcttctacccagtggttccacaaccagagctccacctggggccaggtccaagccagctacacgtttaaggccacagtcaatgacagtggagaataccggtgccgaatggcgcacaccagcctcagcgaccccgtacatctggaagtgatttctgactggctgctgctccagacccctcaactggtgtttctggaaggagaaagaatcacattgaggtgtcacggctggaagagcatacagctggccaggatttcattcctccagaatggagaatttgtgtcatttcatccttataatgttagctactccatctcaaacgccaaccacagtcacagtggggactactactgcaaagcatatctaggaaggacagaacacgtgtccaagcctgtcaccatcactgtccaaggttcagcaacggcgtccaccagctctctagtgtggttccatgccgctttctgcctagtgatgtgcctcctgtttgcagtggacaccggcctgtatttctgtgtacggagaaatcttcaaacctcgggggaggactggaggagatccctgtcagtcggaaagtacaaggctccacaggacaaatgacatcccatcgtatggctaaacagcggcagcttcttttcagccacaccgccacttcatctacagccttcctttgaaagcaacttaccaccaggctggatatttggtcctgcaatcaccaactgctaggaggctgaggcaggaggatcaccaaaggctgcccgggttttagagaagagagagtgcaagtctatcctggataacccaataagaccctgggtttagatggcttatcaggaaagagcacccattgctaagctcaccagacaagaccacagattgatccctaaggacacagggcaaagggagagaacaaattcctgaaagttgtcctctgacctccacaggtccaccatggcagatgcacacaataaacaaattaaaatg

SEQ ID NO: 12 - protein, 267 aa, Rattus norvegicus
MTLETQMFQNAHSGSQWLLPPLTMLLLFAFADRQTGDLLKAVVKRDPPWIQVLKDDTVTLTCEGTHNPGNSSTQWFHNQSSTWGQVQASYTFKATVNDSGEYRCRMAHTSLSDPVHLEVISDWLLLQTPQLVFLEGERITLRCHGWKSIQLARISFLQNGEFVSFHPYNVSYSISNANHSHSGDYYCKAYLGRTEHVSKPVTITVQGSATASTSSLVWFHAAFCLVMCLLFAVDTGLYFCVRRNLQTSGEDWRRSLSVGKYKAPQDK

SEQ ID NO: 13 - DNA, 1396 bp, Rattus norvegicus
  CDS (33)..(377)
ccccagaccagctgggcctgtccttcaggatgatgagacattccaagactgccatttcattctttatccttgtggccgtccttgggtcccaggctgggctcatacaagatacggtaaaagaagatcgtccctttaatcctacaataattcaccaaggctttcaagactcttcagactgctgcttctcctatgcctcacagatcccatgttcaagatttatatattattttccaaccagtggtgggtgcaccaagccaggcatcatctttgtcaccaggaagaggaagcgggtctgtgccaacccgagtgatcagagagttcagacgtgcataagcaccctgaagctaggcccaagatctgggaacagtgccattgcttgagaaggagggcgtactgccacctgcattcttctgtctccccagtgaccacctaggaagcgctaagcatttgtttttatagatatttaaagcacgtattcttctgtacaggtttaaagcagcacatgtagtccacagtattcacatggattcagcctgacatgattagagccatctcgaggtgtaaccatcatgagtgtctttgggtaactgttgggttttcttgcatagttcctcagcagattataaatggataaactactagtctttgggactttggaatgtgtctggttttgatgcaagcttaagccaggtgtatccagctgagatgaaatcaatttttgccctaagccatacatatgtccaactttgtggggacccagctgttcttcctgccaccatagagcagtaagtgaccccaataaagtgcactccatgtagctatgggtctgtctctcttcagattcatagggctgtggggccagtctgaaatcgggctattagaatgaaagtatcccactcactcgctcgctcaccttgaatacagcagggagaaagggatcatataactgaatattctaaatgatataaatttctaatatataatatttctaaatataatttctaaatattctaatgatagaaattttaaaaaaaaattaaaacacagttttttccgatatattcctctgagtttggtgcaaaatggaagaatgcctgttccagcagggcatcttcttctctacctgaccttctctggggaacctgacccccaaaccctctaccaaaggaagttcagatagctgtcgacaaattgttcaacttcctttttccagcaattacctgggattcccggaatggctctccatgcaaatgaggcattcccaatactttaaacttcatccaataatttacattttaccccgaaaactcctacccacactccaaggttcatgtataggcctggttcaccccaaataaagtgtgcatgcgcaaaaaaaaaaaaaaaaaaaaaaaaa

SEQ ID NO: 14 - protein, 115 aa, Rattus norvegicus
MRHSKTAISFFILVAVLGSQAGLIQDTVKEDRPFNPTIIHQGFQDSSDCCFSYASQIPCSRFIYYFPTSGGCTKPGIIFVTRKRKRVCANPSDQRVQTCISTLKLGPRSGNSAIA

SEQ ID NO: 15 - DNA, 604 bp, Rattus norvegicus
  CDS (8)..(364)
cggcacgatggctcgctcggtgaccgtgatctttctggtgcttgtctctctggccgtcgtgcttgccattcagaaaactccccaaattcaagtgtactctcgccatccaccggagaatgggaagcccaacttcctcaactgctacgtgtctcagttccacccacctcagatagaaattgagctactgaagaatggaaagaagataccaaatatcgagatgtcagatctgtccttcagcaaggactggtctttctacatcctggctcacactgaattcacacccaccgagaccgatgtatatgcttgcagagttaaacacgtcactctgaaggagcccaaaaccgtcacctgggaccgagacatgtaatcaagctctatggagctctgaatcatctggaccagtttaactccagatccggtttctaatatgctatacaatttatccacaaagtaaagaatagcaatgagcacaccatcttcttcatatcttaccttaaatattttatgcatgttttaaaaaaaattggagactaatatcctagatttccggaataataaagcttcaatgagtgttttgatcagaataataaatatggttaagaac

SEQ ID NO: 16 - protein, 119 aa, Rattus norvegicus
MARSVTVIFLVLVSLAVVLAIQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 17 - DNA, 1067 bp, Rattus norvegicus
  CDS (111)..(710)
acggaggaccgtcagccagcaatcgcgtagctcgatcgatcacccaggaagagaaattgtaagcagataagaagaacgcgttcggtttgggacaccatttgcagctggaaatggggaatggactgtcagaccagacttccatcctatccagcctgccgtcctttcagtcctttcacattgttattctgggtttggactgtgctggaaagacaacagttttatacaggctgcagttcaacgaatttgtaaatactgtacctaccaaaggatttaacactgagaaaattaaggtaaccttgggcaattccaaaacagtcacttttcacttctgggatgtaggtggtcaagagaaattaagaccactgtggaagtcatatacccgatgcacagatggcattgtgtttgtggtggactctgttgatgttgagagaatggaagaagccaaaactgaacttcataaaataactaggatatcagaaaatcagggagtccctgtgcttattgttgctaacaaacaagacctgaggaactcactctctctctcagagatcgagaagttgttagcaatgggtgaactgagctcatcgactccttggcatttacagcccacctgtgcaatcataggagatgggctaaaggaaggacttgagaaactacatgatatgataattaaaagaagaaaaatgttgcggcaacagaaaaagaagagatgaatggcagtacttttatatcggtgtggaataggttttacttggtctgatttctgacaagctgaagagtgtctacagcctggtttgcctgtctgccctcacggatgctattaaagctttgttttgttgaacagtcagatacccaactctgttgccttgtggaagatgagtaaatgcaatgcttcttaaaggggtctcttctccgtgacccacaaatcttttggtactaccattttgggaagccaaaaaaggctagtaattgaccagaaaacaattttgtggaaatttgacctgaagttagtgaaataaaactttgaagagtgtaaaaaaaaaaaaaaaaaaaaaaaaaaaaaaaaa

SEQ ID NO: 18 - protein, 200 aa, Rattus norvegicus
MGNGLSDQTSILSSLPSFQSFHIVILGLDCAGKTTVLYRLQFNEFVNTVPTKGFNTEKIKVTLGNSKTVTFHFWDVGGQEKLRPLWKSYTRCTDGIVFVVDSVDVERMEEAKTELHKITRISENQGVPVLIVANKQDLRNSLSLSEIEKLLAMGELSSSTPWHLQPTCAIIGDGLKEGLEKLHDMIIKRRKMLRQQKKKR

SEQ ID NO: 19 - DNA, 2524 bp, Rattus norvegicus
  CDS (136)..(720)
ccctagtttccctccccgggccgcaggagaaacagtgccctctcccttcttgacccctggcccttccctcccttattccctcttgccgccgctgcttctggcgctgccgctcccggaggagctcccgtcacggtgatggggtctcgggcctccacgttactgcgggacgaagagctcgaggagatcaagaaggagactggcttttcccacagtcagatcacacgcctgtacagccgcttcaccagcctggacaaaggggagaacggaactctcagccgggaagatttccagaggattccagaacttgccatcaacccactgggggaccggatcatcaatgccttcttctcagagggagaggatcaggtaaacttccgaggattcatgagaactttggctcatttccgacccattgaggataacgaaaagagcaaagatgtgaatggaccggaacccctcaacagccggagcaacaaactgcactttgctttcagactttatgacttggataaagatgacaagatctcccgcgatgagctgttacaggtactgcgaatgatggtcggagtgaatatctcagatgagcagctgggcagtattgcagacaggaccatccaggaggctgaccaagatggagacagtgccatatcttttacagaatttgttaaggttttggagaaggtggatgtagaacagaagatgagcatccgatttcttcactaaagggcagagaccaaattattcttgctttctagtatttaagaactgaaacttccttctgtcctccagccccaccccatcctgtcatccccctttcccagaatactacagctcctgcatgacaacctgaagtctctcctgtccatacaaacttgcctttggtgtataaacagggcgttacagaatggtaccctgtctatttctgttcggtatccacccatcagtctttattgtaagtaccaacttcccctgttctactgctcagtgccacgcatccgtcctgtctgaggaaagaaacagggactcatgtcaagaaccagccagcaaagctcccactagatgggaccatatccatgctgccttcccttcatcagccctccatcatggtcctgtgtgctgtgtcctgtgtcctgtgtctgtttcttcctacctctctgtcatccagccttctctccattcagtctttcagctgtcctcctgtcgttgagattccctgcattctacttgactctgggctctgctggtaagcctgtttctgatgtggcaggatagttagtggtctggtgatctgtatctaccttctgatgtgggatattgcctatccaggagctattgctctgtcgtcacagggcttatgagcagacatgttcttaaaaggtagtcatggtgaggcttgtagagccatgtctcctaggtggcttcccaggtctctctacctctgactttttattttcaaatatatttatgtggcttttctgggtacagtgttgacattcagaaaatagtctaatgcctgccagtttgcttctctttgggacattgtcaccacccaagcggctgttctgacaatatggtagagggccttcttgtgttttctgacaaggatgggcaccctggagagctgcccacatttgctccacagcatgggttatgcctcagtgttctgtacacttgaagattcttcacatttccaaacagaaagactagtgttacagaagtgcctgacttacctcagtcctaaagaattagattgtcctcagtgtcttaaagttttgcacaaaactctgaatgaattttatacttggcaaattttaatgctggaagatgtatagtcagttcataatcctggtccaggcactgtatgcatcgaatgtattgtctcatgagcttcctaatgacacgaccttcctagctccttgtgaatactatactatgcagatccctcagaagcatgggacctgggactggtggcaccaactgaggtgacattctctgccaatgtcagcttgatttagatgtcactgaatcaagctgcttccgccctgtgttcctgtgaatctgctcttggctggctttctctggtgttgcctgcttgcttgcttgctttgttgctttggaaggctatccaagatatgtaagcaattctttaggaaataaattgctgttaacccaaacaaacaaaaacactgcagacgagatgggtaaaggcgagcagtgggggcgtggctaggacagatggctagggtctacatgaactggggaattctttatgaaatatgtcagactttgtgatcaaatgatgtgatataagaattgtataaagggaataattgtctgatacttatctgtttgagaagtacttcagaagcatcttgatttaattatttaaagtttctaagattatgccccccttggctgtatagcaaactgtttttatccacagttgtgccttattgtcccattaaaattgtattcctcaatccaccaataaattaattgtcgttaaaacaaaaaaaaaaaaaaa

SEQ ID NO: 20 - protein, 195 aa, Rattus norvegicus
MGSRASTLLRDEELEEIKKETGFSHSQITRLYSRFTSLDKGENGTLSREDFQRIPELAINPLGDRIINAFFSEGEDQVNFRGFMRTLAHFRPIEDNEKSKDVNGPEPLNSRSNKLHFAFRLYDLDKDDKISRDELLQVLRMMVGVNISDEQLGSIADRTIQEADQDGDSAISFTEFVKVLEKVDVEQKMSIRFLH

SEQ ID NO: 21 - DNA, 913 bp, Rattus norvegicus
  CDS (227)..(646)
aagctgctaccggcgcctaaccgttccccgcctcctggacgtcggctcttcaggcggagcctctaggaggaagaggagaacaagagagaaactcttctggcccgggaccgcggcccgggcgggacccggggccgagtcgcctaggcgacgttgaccgagaccgagcagtcactggatccctaaggaggcggcattctggcagaggcctctggcagcagacccagagatggcatcagtagatttcaagacctatgtggaccaggcctgcagagctgcagaggaatttgtcaacgtctactacactactatggataatcggcggcggctgctgtctcgcctgtacatgggcacggccactttagtatggaatggaaatgccgtttcgggacaagaatccttgagtgagttttttgagatgttgccttccagtgagttccaaatcaacgtggtagactgccagcctgttcatgatgaagctacgccaagccagaccacggtccttgtggtcatctgtggaacagtgaaatttgagggcaacaaacagcgggacttcaaccagaacttcatcctgactgctcaggcgtcacccagtaacacagtgtggaagatagcgagtgactgcttccgcttccaggactgggccagctagtgggggtgacaaaggcagcttggctttagccaagctctgcagagaaatgccagtctcaaatctcaggacgtggagaacactagttcatttttgttgtcacagcagcactgcaaactggatgttcgactcctagaaacccctttcctggttatatactggtttgtcatagttgccttttaaaagtagtaaactttctatttttctacttactcagtagagaacctgtttctggaaattgacaaataaatatgctgcttctctgcc

SEQ ID NO: 22 - protein, 140 aa, Rattus norvegicus
MASVDFKTYVDQACRAAEEFVNVYYTTMDNRRRLLSRLYMGTATLVWNGNAVSGQESLSEFFEMLPSSEFQINVVDCQPVHDEATPSQTTVLVVICGTVKFEGNKQRDFNQNFILTAQASPSNTVWKIASDCFRFQDWAS

SEQ ID NO: 23 - DNA, 2623 bp, Rattus norvegicus
  CDS (61)..(867)
gagctcttggagggagttgagcaccaggcatccagcggttatgtcaaggcccctgccaacatggccagcgaaatcaagaagaagctcttctggagggctgtggtggctgagttcctggccatgaccctcttcgtcttcatcagcatcggttctgccctaggcttcaattacccactggagagaaaccagacgctggtccaggacaatgtgaaggtgtcactggcctttggtctgagcatcgctactctggcccaaagtgtgggtcacatcagtggtgctcacctcaacccagcggtcacactggggcttctgctcagctgtcagatcagcatcctccgggctgtcatgtatatcatcgcccagtgtgtgggagccatcgttgcctccgccatcctctccggcatcacctcctccctgctcgagaactcacttggccgaaatgacctggctcgaggtgtgaactccggccagggcctgggcattgagatcattggcaccctgcagctggtgctgtgcgttctggctaccactgaccggaggcgccgagacttaggtggctcagccccacttgccattggcttgtctgtggctcttggacacctgctggccattgactacactggctgtgggatcaaccctgcccggtcatttggctctgctgtgctcacccgcaacttctcaaaccactggattttctgggtgggaccattcattgggagtgccctggcagtgctgatctatgacttcatcctggccccacgcagcagcgactttacagaccgcatgaaggtgtggaccagtggccaagtggaggagtatgacctggatgctgatgatatcaactccagggtggagatgaagcccaaatagaggaggcttggcctgggcatccgtatgggggtggggcagggatgggcggagggaggggagggctgaactcatattatagacactctgacaagctgaccaaagtcactccccaagacctggctgtcccacatggtctagccttgtctggggcagtaatatcactgtctttctttctgtttcctggtctcagagcttcctggggcccaagacttatcaactcagcacctcactcctttgacatcatggaggagtgaaagggagggacccacctgctagttgtcccctcagagtgtgctagaaagtccccctcactccaaagttgcccagcaagacacccaccttaggtgcctgggattctgccattgttactttgtgcctttgatcatggccttccttaggtaggcaccttagccccaaaggcactttgagggagaggaagtcccttagctttcccctttggtctgacttacctccaggacccttccccttaaactcactctaagaccttggaattttccctatgctgaggcctcagtgatcacgtctgtaaagtggcaaggaagggacagctttggggactctgtaagtgtggacaagccacaggttctagaagggagagtctagacagcactgcatagtgcacctggctgagctctttcccaacatccacagcccatgagcacgtgtggatgagctacagagcggagaagcagtccaggatgctgggagtgcctgttcacacagagagccttcctttaggcaggtctgttgcagcagagtaaaggtcatttcatggtggaggaagagctcacgaaatctctcctagctctagtgggtctgctcatgtaccatggcaccaactggctgtttccctctagtctcccttgcagtgtagatgtgacgtgtgtgtttattaaagagcactgggctattgcagcgtcatgtctgaggaaagaagcagctagacatgcaacagaccccagacagatgcccatgccgggcacacaggggtttggatgcccctagttcatcatgatcaaggaggccactcttgtcctggcttatgagctgctggctggatgaatttgaccagagcctggacaatctgaaggggctcactatgtgactccaggcacagtctccttattgcaaggacctgatgctgtggcttctgctatagcccaagaacatctcagagtgcattcagctcagggcttgcatttagctctctgggttattctattcaatcccaccaagccagagcagctctaccactgtgccgttaaccatgtcgtgaaccgagagccacattcttcaggtgcttagaagcagcagaatagtcaggaggccattgaccactggcatagtacagctgcatactcttctccattccctagcaggcactatactcacttcacaggtcaggacactgaggacccatatgctcaactatatcatattgggtcaagctcagcagtcaaagccatgtttggccacttcatagtcaatgaagtggactcctctcatttccagcttctccgtttctgcctggacaatggctaagggccagggatgggggtaggggtgggagtggaggtggtgagagctgtgctggaggggggcaggcaggcccatcccacctggctctggccactgtgaaccaggaccctgcatctgtctgcctgtgtatgtttctttgcaattggaattccatcttatggtaagaaaataaaggacaatgacttgtaaggtc

SEQ ID NO: 24 - protein, 269 aa, Rattus norvegicus
MASEIKKKLFWRAVVAEFLAMTLFVFISIGSALGFNYPLERNQTLVQDNVKVSLAFGLSIATLAQSVGHISGAHLNPAVTLGLLLSCQISILRAVMYIIAQCVGAIVASAILSGITSSLLENSLGRNDLARGVNSGQGLGIEIIGTLQLVLCVLATTDRRRRDLGGSAPLAIGLSVALGHLLAIDYTGCGINPARSFGSAVLTRNFSNHWIFWVGPFIGSALAVLIYDFILAPRSSDFTDRMKVWTSGQVEEYDLDADDINSRVEMKPK

SEQ ID NO: 25 - DNA, 2070 bp, Rattus norvegicus
  CDS (1)..(1113)
atgacgtaccagttaggagctcggatggatcagaaactgagccccagggcccgcactccgggcagtatctccatcaatttaaggctttgctcaggcaccatggtccccatctcctcggtggccattacactgaggtggtcagtgcctgacaagccttgcaccgtcccgaacctgggcgcggctcaccaggtgccggacgcccgcgcttccgctaagccccgccccccagaccaatggcgcggcgactgcgggggcgacgagctcccattggtcgagagggcgcggcggtgggcgcccattggcagagctgcggaggggcggggccaagtgctccatggccccggcctgcgggcggcgctcagcgctcgctgcgaggcgcgggcggaagtggcgacgctggcgtcccgggaggcggcgggcggcgacagcccgggcgcggacgccctcggcgcgggctggggcatcaccaagggcctcgaccccgaaatggcgctgctggccgagcacctgctcaagccgctacccgcagaccggcagatcgagaccgggcccttcttggaggcggtggcccacctgccgcccttcttcgattgccttgggtccccggtgttcacgcccatcaaggcagacataagcggtaacatcaccaaaatcaaagccgtctatgacactgacccggccaagttcaagactctgcagaacatcctggaggtggagaaggggatgtatggggcggagtggcccaaagtgggggccacgctggcactgctgtggctgaaaaggggcctgcgcttcatccaggtgttcctgcagagcatctgcgatggcgaacgggacgagaaccaccccaacctcatccgggtaaacgccaacaaggcctatgagatggcgctaaagaagtatcacggctggctggtgcagaagatcttcaaggccgcactgtatgcagcaccatacaagtctgacttcctaaaggcgctctccaaggggcagaacgtgacggaggaggagtgcctggagaagatccgcctcttcctggtcaactacacggccaccatcgacgccatctatgaaatgtataccaagatgaacgcggagcttgactacaccgtgtaggcgcccagacacacagccccacgtggacacacagcccacatggacacacagcccacgtggacacacagccccacgtgggcacaagcagccccatcactgtgaaacaagctgggatccccagctcagcctggcccgcctggccagcgtgggtctgcagggggcggccttagcttgttagaggtcttttgttttcttgtttttgttatcttggccttttctaatggagcaataaacaccctccactcactccagcttccccagcagctcaggctgaaccaacctccttccagtgtggttaaaaccaattaactttgctaacgatgaattcctagggttttgtctttccctcccctacctagctcaggaggagcccaactcaggggagacatacacctcacccagcaaagaagccaggtccatccggcccaagggtgtcttggatgtttgaaggacaatggaggcggatgggaaattcagcagggcccaactgtggtgtctgtttaaaaggcgagctgacagggtcctcacggggctgcacatctggcattaccagccctgttctctctctccctctgtcttgtcagacaggtttttttgtttctcctgaattgttaccagcctttcaaatgaccgcctaggcacacacaggcgaccgtccgtgactgaactcgcattcttctgcataatgctttcttcctcagggttggctcttccaacccaagaaactgcctgtcacggggccctccagctgcggagacccaagaaactgccttgctgccttaaaccgtccttcaggagctaaaagagctatattttttttaaagtatcggggttaaacacatcctgaaagaattgatgtatgttaaaaaaaaaaaaaacccaatgaggaacaattggtgatttttatgcagaaactaaataatccttaataaataaatctatattttgaaatca

SEQ ID NO: 26 - protein, 371 aa, Rattus norvegicus
MTYQLGARMDQKLSPRARTPGSISINLRLCSGTMVPISSVAITLRWSVPDKPCTVPNLGAAHQVPDARASAKPRPPDQWRGDCGGDELPLVERARRWAPIGRAAEGRGQVLHGPGLRAALSARCEARAEVATLASREAAGGDSPGADALGAGWGITKGLDPEMALLAEHLLKPLPADRQIETGPFLEAVAHLPPFFDCLGSPVFTPIKADISGNITKIKAVYDTDPAKFKTLQNILEVEKGMYGAEWPKVGATLALLWLKRGLRFIQVFLQSICDGERDENHPNLIRVNANKAYEMALKKYHGWLVQKIFKAALYAAPYKSDFLKALSKGQNVTEEECLEKIRLFLVNYTATIDAIYEMYTKMNAELDYTV

SEQ ID NO: 27 - DNA, 882 bp, Rattus norvegicus
  CDS (56)..(652)
ggctcacggttggttctgtttgtgagacctgtagctcgactctgctgatagcaagatgtcttcaggaaatgcaaaaattgggcatcctgctcccagcttcaaagccacggctgttatgccggatggacaattcaaagatatcagcctaagtgattacaaaggaaaatatgttgtattctttttttaccctcttgactttacttttgtgtgtcccacggagatcattgctttcagtgatagagcagaagaatttaagaaactcaactgccaagtgattggagcttctgtggattctcacttctgtcatctggcatggattaacacacccaagaaacaaggaggattgggacccatgaacattcccttggtatcagatcccaagcgcaccattgctcaggattatggagtcttaaaagctgatgaaggtatctctttcaggggcctctttattattgatgataaaggtatccttcgccagataacaataaatgatcttcctgttggccgctctgtggatgagattctgagactagtccaggccttccagttcactgacaaacatggtgaagtgtgcccagctggctggaaacctggcagtgataccatcaagcctgatgtcaataagagtaaagagtatttctctaagcagaagtgagcactggaccagttttctggcagacagctttgagcagccagaagaaatttgtactctacacatgacgtggtgtgattccagataagcctttcctacaagggctaggggtggttagcctttcttccactattggtaaggggcagaccatcttatatcagtcacagaaaccaacctgttaattctctctctctcttttttttttttaagtatctattaaacgtgaattc

SEQ ID NO: 28 - protein, 199 aa, Rattus norvegicus
MSSGNAKIGHPAPSFKATAVMPDGQFKDISLSDYKGKYVVFFFYPLDFTFVCPTEIIAFSDRAEEFKKLNCQVIGASVDSHFCHLAWINTPKKQGGLGPMNIPLVSDPKRTIAQDYGVLKADEGISFRGLFIIDDKGILRQITINDLPVGRSVDEILRLVQAFQFTDKHGEVCPAGWKPGSDTIKPDVNKSKEYFSKQK

SEQ ID NO: 29 - DNA, 494 bp, Rattus norvegicus
  CDS (86)..(400)
gggggcaagctccgttgggtgcctaggatccatttccatcgggttctgccgaaactcgtgtggctccctccccgcaacagccaaaatggtgaagctgatcgagagcaaggaagcttttcaggaggccctggccgctgcgggagacaagcttgtggtagtggacttctctgccacgtggtgtggaccttgcaaaatgatcaagcccttctttcattccctctgtgacaagtattccaatgtggtgttccttgaagtagacgtggatgactgccaggatgttgctgcagactgtgaagtcaaatgcatgccgaccttccagttctataaaaagggtcaaaaggttggggagttctctggtgctaacaaggaaaagctcgaagccactattacggagtttgcctaatcatgctctgaaaagtgtgaccagctccagcaggttaaaacctgtaccttttttaatttgcaaaaaaaaaaaaaaaaaaaaaaaaaaaaaa

SEQ ID NO: 30 - protein, 105 aa, Rattus norvegicus
MVKLIESKEAFQEALAAAGDKLVVVDFSATWCGPCKMIKPFFHSLCDKYSNVVFLEVDVDDCQDVAADCEVKCMPTFQFYKKGQKVGEFSGANKEKLEATITEFA

SEQ ID NO: 31 - DNA, 974 bp, Rattus norvegicus
  CDS (293)..(889)
aattcggcacgagccaaccttgagcatctgatcctcttggcttctccttcctatcgctgagctggtaggctggagacagttgtttgggtgccaacatcaacaaacgatttctgtagtttagcgtttatgaccctggcgtgaagatttaaggtctggattaagcctgttgacttctccagctacttctaaatttttgtgcataggtgctctggtctctgttgctgcttagttcttccagccttcctcaatgccagttttataatatgcaggtctctcccctcagtaatccaggatggctactctgatctttgttgataaggataacgaagagccaggcagccgtttggcatctaaggatggattgaagctgggctctggtgtcaaagccttagatgggaaattgcaggtttcaacgccacgagtcggcaaagtgttcggtgccccaggcttgcctaaagccagcaggaaggctctgggaactgtcaacagagttactgaaaagccagtgaagagtagtaaacccctgcaatcgaaacagccgactctgagtgtgaaaaagatcaccgagaagtctactaagacacaaggctctgctcctgctcctgatgatgcctacccagaaatagaaaagttcttccccttcgatcctctagattttgagagttttgacctgcctgaagagcaccagatctcacttctccccttgaatggagtgcctctcatgatcctgaatgaagagagggggcttgagaagctgctgcacctggaccccccttcccctctgcagaagcccttcctaccgtgggaatctgatccgttgccgtctcctcccagcgccctctccgctctggatgttgaattgccgcctgtttgttacgatgcagatatttaaacgtcttactcctttatagtttatgtaagttgtattaataaagcatttgtgtgtaaaaaaaaaaaaaaaaactcgagagtac

SEQ ID NO: 32 - protein, 199 aa, Rattus norvegicus
MATLIFVDKDNEEPGSRLASKDGLKLGSGVKALDGKLQVSTPRVGKVFGAPGLPKASRKALGTVNRVTEKPVKSSKPLQSKQPTLSVKKITEKSTKTQGSAPAPDDAYPEIEKFFPFDPLDFESFDLPEEHQISLLPLNGVPLMILNEERGLEKLLHLDPPSPLQKPFLPWESDPLPSPPSALSALDVELPPVCYDADI

SEQ ID NO: 33 - DNA, 3069 bp, Rattus norvegicus
  CDS (1)..(1755)
atgggaaagggcttcctgcagtgcttctctgcttggcttcctggaaaaactggagctcgagtcaaacaaggggcagttgtgaccgatgtctatgtcatacccgcagccatgagactccagcacaagcagatggtgcttccaacagttgtctacgggggggagtgggtttccccggagccatgggcatccatcctggggttcctgacaaggtccagcttcacactgttttttttcttcactgttcagcgagcaccagggctttttggactctctagaaatggctcctcggttccaccacaaccatcagctttgtgtggtggagcattcatcgcagtatccacccttcaacacaagagaaggtcacaagttcgaagcctctggaacattccatttgggggatccctcgttaccacacagtctaatggcaacaaagcgtttggtgtgctcattgtagtgaaaggaccatcaagtgccaagcaagtaggagccccgcagatgacagttgtgaggagcagcttgctaaggaaccgtcagcgttatgtgaaggaaggaagcattgagtttcaagcccagcgtgatgtcaggccaatgactaaagaatacaaagaaggtgcaggcttcttgtgtcctacagactctcaactccagagtagtgctttccgccttgggatcgtgcgctgtgatcacctcctctccctgagcgatgcacgttgtcctgagatcataaagagttacaaggagattccaatagaaccgggaatgccttgtggccacaagaagtggaggtgggttttggcctacacgcctgctacccacacttgctcacatgactggcacacagcggaagaaagctaccccagttacgtaacgaagcccaagaaggccacgtgtcaccagattaacaccagcgaacccttacaagagcagatcacaagactcagagaacatatgcatctggaaactggccagcactggagccacgccccctccgggctgctcctctctgattggccttctcgggggggcggaaccctagcgtgcaatagttacggcggcagagtttggtcgcaaagcaggtgctctgcagtcagcgccatggctgcccgcgctcctgagctcgcgcgctccctgcttgctgcccttctggctcccgcgctagtggcactactggtgtcgccggcctggggacgcggaggccgggaccacggggactgggacgtggacaggcgactgcctccactgccaccccgcgatgacgggccgcgcgtggcccgcttcgtgactcacgtctcggactggggctcgctggccactatctccacgagagaggaggtgcgcggcaggcccttcgcggacatcatctcaattagtgacggtcctccgggagaaggctcaggcgagccctacatgtacctgagtccgctgcaacaagcagtgagcgacctgcagggaaatccagaggctacgctgactatgtctttagcacagactgcctactgtaggaaccatggatttgatccccagagtcccctctgtgttcatataatgatgtccgggactgtgatcaaagtaaatgagacagaagagggctacgcacgggactcgctattcatcagacaccctgagatgaaacactggcctcctagccataagtggttctttgctaagttaaaaataagcaatatctgggttttggactactttggtggaccgaaagtagtgacacctgaagaatattttaacgtcacactgcagtgaagcaaatacggtgaactggatcacaccagcgacaggtcttccaaggctcatgcacacagagggctcgaccctgcctggctgctagcccactctgccacctcttgtttcgatttcctttgcttgtctttgcagagtgttgtccattagaagacgtgcttgctggatttaaaaaaaaaaaaaaaagtcttgaagccattttcagagaaaaatgcccttctttatgatcaaggaagccatgctcggtgctttttcctcagtggtgccctcagtagggtgacatataccagctgtagaccgcccattggcactggaggtccagaggtgcagatgctctgtgcctgatggccactgtgcctgaaggtgctgtgctcacccagctgcctcaggagccagtgcagaggggccgtatgaatcctccagtccaggacagcagtgaccacggatgcagcgtcccattcacgtcaacctgtatagctgatcagcacaaggcaaagctgggtcttctgtaaaactccataaacgtcttacattctaaagtcatcgttacccagaacaaaataaatcagaaagattttcctgcatccattctccatctaagtagactgtacactcactcctcatccttcggaatgtgcacgcctgagctggggcgcagctttcttggcacagagcttgccaagcatgaatgggaccataacgtcgaaacaaccagccagccacagacgacatctttatgtgcatgaaaaagcaacctcagaagaaaaaggatttcgcatatgttgggctctttttcgtaagacttaaccctgtgccagccgcaatccaagccctcctccggcattctgtatcttgaagctcctggtgctgccgcctgcgtcctctgtgggatgtaccacggtactgtggcataaaaacaaactacaacaacaacaaaacatgtctctgcaccagaggatggctcagcttgtaagatgactgctctagcagaggacccaagttcagtacccgcaaggggtcagacaggcacatatgcccagaactccagctccaggagatcaaatacctctagcctccaaggtcacctgcactggaatacacacacccacacatagacacacagcgacacaatttcaaacagtacatcttttattccaaagaggtactacttagtttaattacctgtttcatgctcttggatgacataagatttcttttaacagagtgtgggggaatcaagacactgacttaaacagccaaaaaaaaaaaaaaaaaagttaactatgaaccatcatgttggagaaagagatggta

SEQ ID NO: 34 - protein, 585 aa, Rattus norvegicus
MGKGFLQCFSAWLPGKTGARVKQGAVVTDVYVIPAAMRLQHKQMVLPTVVYGGEWVSPEPWASILGFLTRSSFTLFFFFTVQRAPGLFGLSRNGSSVPPQPSALCGGAFIAVSTLQHKRRSQVRSLWNIPFGGSLVTTQSNGNKAFGVLIVVKGPSSAKQVGAPQMTVVRSSLLRNRQRYVKEGSIEFQAQRDVRPMTKEYKEGAGFLCPTDSQLQSSAFRLGIVRCDHLLSLSDARCPEIIKSYKEIPIEPGMPCGHKKWRWVLAYTPATHTCSHDWHTAEESYPSYVTKPKKATCHQINTSEPLQEQITRLREHMHLETGQHWSHAPSGLLLSDWPSRGGGTLACNSYGGRVWSQSRCSAVSAMAARAPELARSLLAALLAPALVALLVSPAWGRGGRDHGDWDVDRRLPPLPPRDDGPRVARFVTHVSDWGSLATISTREEVRGRPFADIISISDGPPGEGSGEPYMYLSPLQQAVSDLQGNPEATLTMSLAQTAYCRNHGFDPQSPLCVHIMMSGTVIKVNETEEGYARDSLFIRHPEMKHWPPSHKWFFAKLKISNIWVLDYFGGPKVVTPEEYFNVTLQ

SEQ ID NO: 35 - DNA, 3974 bp, Rattus norvegicus
  CDS (131)..(529)
tcgactacccatattccccaccagccaaccggttcctcaccaagatgtggcacccaaacatctatgaggtaagacactagaggcctgcttcaggcacactccaccacacacttctctgcctcccttgcacatgctccacccctcctctaaccctggtcactcttgcagacaggggacgtatgcatctccatccaccatcccccagttgatgacccacagagtgggagctaccctcagagcggtggaaccccacgcagaatgtcaggtgagaattgccatggaactgcaccgtgggcacccgcaaacaccctgtaccctaagatccacccttccctgctccagtcaggggttcccagagtctggggagctgagcttgcacctacggccctacaggaccatcctcctgagtgtgatctccctgctgaatgagcccaacaccttctcgcctgccaacgtggacgcctcggtgatgtacagaaaatggaaggagagcaaggggaaggaccgcgagtacacggacatcatccggtgagtgcttcctactgtgaagagtgatctggggtgtgggtggggtggggacttgttgtatgtttccattttttgcatcccgggctggatgtgtggaggactctggggcccccagagcacggagttgaccacccagggtggccaagatcccctctgccttgttgttgacttaactccatgagcaactgggctctctgggccacaccctcactgtctgacagagagcatcaatcacttgcatttcgtgcttctgaaacttagagcttttttttttttttttgcaaagtgggaagggggctgagcccactgtgagtctgtcctcagttagggaccagggctgcgacctgttgctgtctatgagtggcagtgtccacacctatcatcgcttcttagcaggagctcctggcctgtctggggccaggcagtctcccctacacccctcacgttgggtatgaagttgcacttgaggggttcagtctttgtcgcttagcttctgtgttttctgagacaggctttcatataactccgtatctccttgtggtccctctgatgtcaggatgacaacacacattcctcggaagtgaccccagtgcttgtctacacagtgacccaaacacagggcctgtcctgtccccaagcctgccctctcagagttggtacaggccgcagctcggtaggcagtgctggtctagcatgtgcacacacacagggttagtctctgtcttaggcaaggtggatgtagccagcatggtccttcatggaggtataagttactcaggaaggtctgagcgctgcactacggaggtgtccccttcaccatggtcaggtcagcaacggtcccggtcactggaagtccctgcagactgtcccctttgttttctgttcagactggctgaacagaactttgttcctcctgctcttcctcagctgagaggaaggctggctgccctagctgttctatatagttctggctgtcctggaagaggagaccctgggtctggccctgtcactgcactctccaccctcctggcagcctttaatccacttctggctgcttcctccctggatccctggcccagagcaggagcccaggctatttttaccattctgactccaggagccacttgctctgaccaggaggcctgtaccactcctggaactcaacctgagccagtatctagtcatgtccctgcctgagcacaaggacaggatagctaggagcctgtgagaaggaccttgagatggatctaggctattgggtgtgctgctgtcccacgtgcccctccaccatagccttgtccccattcccctattccttctgggcagctgaaaacagtgtctgctctgcttgactgcactgggacctgtgccccaagactctggcctgacggtggcctggcctctcaggcacctagttaattgctgcctgccctgtgcaaggaagcccttacatgttgcttcacccatggcctgtgggacctggaacctcagttgttccggaactgcccttcatcatggtgccctcaggtcaggctgcaatttgttgttggtgtgaggaggtgcatgttacctagcccacaactagggtagtgccccccacagtctgggattacagctgtttctgtggtgctgggagctggacatgggtccttatgcttacagaaggagcactctggctgcccctgttgtcggttatagccgaggtcagcctgaatctcatagtagttctcctgcctccgcctccaaggactgggtgacagaccgtctcccacagctggctgtggttacatttctgctggagttggtcactgagccactccttgtcgcctggccctctcctcccacctgctccctgcccccattcatctcatcgtcctgggccgtggcctcacccgccctgtctccccaggaagcaggtcctggggaccaaggtggacgcagagcgcgatggcgtgaagtgcccaccacgctggccgagtactgcgtgaagaccaagcgccggcgcccgacgagggctcagacctcttctacgacgactactatgaggacggtgaagtggaggaggccgacagctgctttggggatgaagaggatgactctggcaccgaggagtcctgacccacgtccctgaaataaacttacaaattttacctcagcaggacccgtgtgggcctcaggcgacagactacctcaccgaggttcaatgtgggctcccatcccatgatccttaggtcctcttaccctgtattccccatgggttttcctcggctttggagaagagctgtggtgcagggcagacaccccactcagccttcagaggatgacaagcctgggaggctgggaaccgactgcccaggggagccagagactccagcccaaagagggccagccccacagtctcctctctgcttttggtgtgtgtgaaatccaaataaaatagctttctgagaagctgagagaagcagccaggacgccggtggcactgggtactgagggctacctgggtacccaggcagggctgtgggatgggcaggaagaggtagcgatggtggcctgtttgttggggctcagtagcatacaggagccccaggcccagaagaaacttgctgcctgcaaatcccacccgcctgcatcgggcactgtggttgcgtccaccccaagcctgtgttcaggaggcctgggtcctgaggcctacaggggattggtattcccaacagtccttgtgttctgggcacttcctcagccctggaaactagtgctgcctgaggagtccctctgatacctgagctgcagcctggtgactccacagcaggggcaggctgagtgaattctctgaccacaccatgctgtgcagggcacctgcagtgaccccgtgctgagatagcagatctgctctgccacctgcatgtttacgggtgctgtggaaagaaagtgtcacttcctcttatgcgggcctgttaatgtccaaagttccagtcaacccatgccagtcagaactgatcagagaaggacacatcggaaaaaggtgttcaggcgatcctctgcccatacaatttgctgccagcctgatctacagttgcaaaaccagcgggaaggctcagtgggcaaaggcctgcaccaagcctagcagaggacccacttggctccaggtggtccctgtgggggttgaggtcagttgagacaaatcccccccccccccccggagttcaccccagtgctaaaaggagcagtgtgccaggagagggagcatcgcttggctgtagttgctgcccccagacacagggcaactttgacagcagagttgctttctaaaaccacacagcagggtgtgaaggaggaggagctgacacgtgggtaaggtgggtggtgcttggaaggccacttgtctccaccggaagagaaagtgtggggccccttcccccagggaccaccagtcctgtgccagg

SEQ ID NO: 36 - protein, 133 aa, Rattus norvegicus
MLHPSSNPGHSCRQGTYASPSTIPQLMTHRVGATLRAVEPHAECQVRIAMELHRGHPQTPCTLRSTLPCSSQGFPESGELSLHLRPYRTILLSVISLLNEPNTFSPANVDASVMYRKWKESKGKDREYTDIIR

SEQ ID NO: 37 - DNA, 1838 bp, Rattus norvegicus
  CDS (169)..(1395)
cagctcctgccccttcagtgacgggcggccaagctgacaggagccggggcggggcggcggcggccagcgaaggagcgcgcggcggcccggccccgcccctggtcctcccgccagccccagtgaccttcacgggttcgggcggcgggtgcaggcccgggcggcggcgcgatgttcgtgcaggaggagaagatctttgcgggcaaggtgcttcggctgcacatctgcgcagccgacggctccgagtggctggaggaggcgaccgaggacacatcggtggagaagctcaaggagagttgcctcaaacatggtgctcatgggagcctagaagaccccaagaatgtgactcaccataagttaatacatgctgcttcagagagggtgctgagtgacagcaaaacgatcttggaagaaaacatccaggaccaagacgtcttgttattgataaagaagcgtgctccttccccaatccctaagatggctgatgtctcagcagaagaaaagaagaaacaagagcaaaaggctccagacaaagacgctattcttcgagcaacggccaacctgcctgcctgtagcacggaccggactgctgtccagaccaccatgagagatttccagacagaactccggaagatccttgtgtctctcattgaggtggcacagaagctgttagcactgaatcctgatgcagttgaattgtttaagaaggcaaatgctatgttggatgaagacgaggatgagcgagtggatgagaccgccttgcggcagctcacagagatgggctttcctgagagcagagcctcaaaggcccttcggctgaaccacatgtcggtgcctcaggccatggagtggctgattgaacattcggaagatccagctattgacacacctctcccaggccatgccgcccaagcggaggcgagtgctgcagctgccacctcctcctcctcctctgaagctgccgtgggaactagtgttgaagatgaggagtcccgggatgagctcacagaaatcttcaagaagatacgcaggaaaaaggaattccgagccgatgctcgggctgtcatttcgctgatggagatgggctttgatgagaaggaagtgattgatgccctccgagtgaacaacaaccagcagaacgcagcttgtgaatggctgctgggggaccggaagccatcccctgaggagttggaccagggcatcgatcccaatagccctctctttcaggccatcctggacaacccagtggtgcagctgggtttgacaaaccctaagacattgctagcatttgaagacatgctggagaacccacttaacagcacccagtggatgaacgacccagagacgggcccggtcatgctgcagatctcgagaatcttccagacactgaatcgcacgtaggtggtgcctactgtgctccttggctgtgcagtctcagcagtctgtgaggcggtgggcgcagggcccagccaggatcttgcctggatcacagcttcagtgactctcctttcctgacctaacggcttgtcatcttctgccacatggtttgaaagttgcagtcccagcttctgacaggtgtttacagagagtggctattgcactgggaaggtggtgtttgctgccccgtcccctccagacactctgctgggctcctgaagtgctacttgaattgacccttaggtgtttgcttgtgtagcccaatgtattttaaagaaggcatgttctgatccaaagtgttatgtcaagtgtgctaatttaactagactgactcacagatggcttctttaataaataaacccttttcctaaaaaaaaaaaaaaaaaaaaaaaaaaaaaaaaaaa

SEQ ID NO: 38 - protein, 409 aa, Rattus norvegicus
MFVQEEKIFAGKVLRLHICAADGSEWLEEATEDTSVEKLKESCLKHGAHGSLEDPKNVTHHKLIHAASERVLSDSKTILEENIQDQDVLLLIKKRAPSPIPKMADVSAEEKKKQEQKAPDKDAILRATANLPACSTDRTAVQTTMRDFQTELRKILVSLIEVAQKLLALNPDAVELFKKANAMLDEDEDERVDETALRQLTEMGFPESRASKALRLNHMSVPQAMEWLIEHSEDPAIDTPLPGHAAQAEASAAAATSSSSSEAAVGTSVEDEESRDELTEIFKKIRRKKEFRADARAVISLMEMGFDEKEVIDALRVNNNQQNAACEWLLGDRKPSPEELDQGIDPNSPLFQAILDNPVVQLGLTNPKTLLAFEDMLENPLNSTQWMNDPETGPVMLQISRIFQTLNRT

SEQ ID NO: 39 - DNA, 1099 bp, Rattus norvegicus
  CDS (16)..(537)
gagctgggccagaagatggcggcagccgccgagctgaggcttctggagaagagtctgggactgaagccggggaataagtacagcgctcagggcgaacgacaggttccagtcctacaaacaaacaatggccccagtctaatgggattgagcaccattgcgacccatctggtcaaggaggccaacaaggagcacttgctggggagcactgcagaagaaaaggcactagttcagcagtggttagagttcaggatcactcgagtagatggacactccagtaaagaagacacccagactctgctgaaggatctcaactcctatctggaagacaaagtctaccttgcaggacataacactaccctggcggacatcctcctgtactacgggctccaccgctttatagttgacctgacagttcaagaaaaggagaaatatctcaatgtgtctcgctggttttgccacattcaacattacccagacatcaggcagcatctgtctagtgttgtcttcatcaagaacagactctatgccaactcccactaggagcccccagcagcaggatagaagtgaaggggaaagtgtgttctggaccatggtactaatgtgaactgatatgacgtcattgtcccttgtgaaagtgtaagctcaagtctgagtgttttacttgtcccttagttgaggctttgtaatttttaatgtaaaagttcagctgttatccaagcaaatcacgatcaaattacaaatctcgctttttatgagcgaagttgattttaataacagttgcaaatcaatggctcaactttaaaaaaacattgctgttggtgtcacatctggctgttcactttttcatgtttttaaaattcatttttctcttaggtatttattgtagtttatttactagaacctacgacaaattatgttgagcaatcagaaagggaagatagatatatttgttctcaaaatatttatgtgaactagtaaatgtggttttaaaaatcaccttttgttatctttcctgtagtcagtgtttactgagccccccactgctagggaattaaatgtgtttgttcctcttaaaaacaaaacaacaacaacaaaaact

SEQ ID NO: 40 - protein, 174 aa, Rattus norvegicus
MAAAAELRLLEKSLGLKPGNKYSAQGERQVPVLQTNNGPSLMGLSTIATHLVKEANKEHLLGSTAEEKALVQQWLEFRITRVDGHSSKEDTQTLLKDLNSYLEDKVYLAGHNTTLADILLYYGLHRFIVDLTVQEKEKYLNVSRWFCHIQHYPDIRQHLSSVVFIKNRLYANSH

SEQ ID NO: 41 - DNA, 4568 bp, Rattus norvegicus
  CDS (1)..(3009)
atggcggcggggggtgctgcggggctgcgggaggagcagcgctacgggctggcgtgcgggcggctggggcaggacaacatcaccgtgctacatgtgaagctcaccgagacggcgatccgggcgctggagacttaccagagccaaaagtggggcgctggctcgggctgctgcggcggcacccgcctaggggagggaggccagccggagtctccgggccgtggtccggggccactggatcgaggatgcgggcgggcggcccctggtggcggggtccgcctggcaggggctgcgcggccgctgcgggactttctgacggcgcccgcggggttcctagcttgtttggtcccgagaagggaccgggtggaggagagagaagtgcggagggggcgcgcgcagctaccaggactcggagacactggcactccgagtgtaggcgtgagtgtacacggcgtacctgggaaggccgggacactggcactgagaggagggggagggggacacaccttgcactctcagggactctgtgccttgggttcccggagactagcagcgtaccatgtggccacaaaccgacaccgcatctcggggagagacccagaagactcaaaaacaactagagcctggaacaagacggcaggttctggggaaaggaattctcagaagctcggcgtagccgaaaggcgcgccgtgtccggtgctgactcgctgtctgttggcgaggagcgagcccgaagaatggaacacgagtccatggaacaatcaggtgtagatgtcagcatgaatacacacctcaaagctgtgacaatgaccctgaagaacagagagcctgtacagttggaaacattgggtattcgaggcagtaacattcggtattttattctacctgacagtttacctctagatacactacttgtggatgttgaacctaaagtgaaatctaagaaaagagaagctgttgcaggaagaggccgaggccgggagtttaattccttttcaaccttcaattcagttccaaggactccaagggctgttaatgagagtgacagtcaggaagacattagagaagtccttttggagctgaatttgaagggttgccaggacttggttggtgagaaaagaggtaaatacgttttgggcctgcgctttcacagcatgaggaaagggcaaacagtctgtcttcaaagaggagaatgggaactcacatttccattggacagtgaacaggtgctggcttgtttaacccaggtgtctgaaagttctgtatttggctataaagatgttcagcttatgaaaattcccaaaaatgatcccttcaatgaagtccacaactttaacttctatttgtcaaacgtggggaaagataaccctcagggcagctttgactgcatccagcaaacactctctagctctggagcctctcagctcaattgcctgggatttatacaggataaaattacagtgtgtgcaacaaatgattcctatcaaatgactcgagaaagaatgacccaggccgaggaggaatcccgtaatcgaagcacaaaagttatcaaacccggtggaccatatgtagggaaaagagtgcaaattcgtaaagcacctcaagccatctcggatgcagtgcctgagaggaaaaggtcaacgcccatgaaccctgcaaatacaattcgaaagatgcatggtggcagcagtgtctctcagaggccgtacagggacagagtcattcatttactggcactaaaagcctataagaaacctgagctgcttgcccgactgcagaaagatggtgtcaatcaaaaagacaagaactccttgggagcaattttgcaacaggtggccaatctgaatcctaaggacctctcctatactttaaaggattatgtatttaaggagctccagcgggactggccaggttacagtgagacagacaaacagacactggatttggtgctctctagaaaattaaatccatctcagaatgctggcactagccggccagagtctcccatgtgttccagcaaagatgctgcctcatctcctcagaaacgacctttggattcagatttcattgatcctttaacgaacaaaaaagctcgaatatctcacctgacaaacagagtcccaccgacattaaatggccatttgaatcccaccaatgaaaaatcttgtgcaagccttctaccgccccctgcagctgctgccatccccaccccctcaccactgccttcaacccacctgcctgtctccaatcctacccagactgtaaattccaactccaattcccctagcactccagaaggccgggggactcaagacctacctgttgacagttttagtcaaaacagtagcatctttgaggaccagcaagacaaatatacctcaaggacttgtctggaggaaacattaccctctagctcagctttactaaagtgtccaaagcccatggaagaagaacatccagtgtctcacaaaaagtccaaaaagaagtctaaaaaacacaaggaaaaggaccaaataaagaaacatgacattgagagcagggaggaaaaggagaaagaccttcagagagaagaaactgccaagctgagtaatgacagtccaaatcccaatgaaggagttaaagaagagtgcacagcttccatggagccttcttcaacaattgaactcccagattatttgataaaatatattgctattgtctcttatgaacaacgccagaattacaaggatgacttcaatgctgagtatgatgaatatagagctttgcatgccaggatggagactgtagccaggagatttattaaactggatgcacaacgaaaacgcctttctccaggttcaaaagagtatcagaatgttcatgaagaagtcttacaagaatatcagaagatcaagcagtccagtcccaattaccatgaagaaaaatacagatgcgagtatcttcataacaagctggctcacatcaaaagactaataggtgaatttgaccaacagcaagcagagtcgtggcactagggctctgcttgaagcagaagatgtgaataaacttaagcttatttatttaaaattccagatgagttgctctggactctaaacgatgacactttgttggacgtttcaggattagtaaacttgagttacttctgttcctctccattttactttgcttccctgaatttgaagctgctttttttctggtcctctccctgacttcccaccccaatctttgttaatagaagcaattttttagagattggagatccagtgtctatgcttcaaatcaattttccttgaaatcttgtgtttgtcattagatatgatttttgctagatatcagggatgcaaagtccacacttaaaagagttgtgtgtgtttaaaacaagacaaaatagtaatgtgcaaggtgataagcttttgaataaacataagcctattttctgacctttcttaatgcaaactctttgccttaaatggtagaatatttaaacatttgcacaaagtacaaaaaaatcaaaacattgtttttaaagggttgaaaaaaggttgtgtgaatgtgtgtagttttctatgcacatatgcatacatacatatatacatatatacacacatacatacatacatacatacatacatacatacatacatacgggctgtctggctccagagtgttaaatctgccctacaagttagacctccattgcaatggctgccttaaggtgtttgctacatcatgtggttaatcagtagctacactgcttcccagtgataacaaccctgggaagcatactgtggcctaccagcatcatcagaaagtgtgtgttctgcttgtatgtctgcagagctgctgtggatgtgagctgcgtgaaggctaagaagctgctgctctcaggccagacactcaggttaaagaactgatgagtattgcagtagcttatttggtgtgtgtgtgtgtgtgtgtgtgtgtgtgtgtgtgtgtgtgtgtgtgtgtggtgtgtttctgtcaggttgctacttctattgtggaagacaaaagcatttccatttcaacagtttgtcagctttattaatgttgggcaaaaaatgatatgttataaaaatgaaacagatcttatagttttggggcaaaatgacaaaaataaatgtgtaggtaatctatttatatactgctataaagtattttttgaagagagatatgcaagaaactattacctacatacatcagatgtatatttaaagatttctttcatcctgcataccaggaatataaaaaagtagatatatataaccataacatactgtgattcatcaaatagcacaaactttcatttcatggattttatctgttaacgttgatttaaactatcacttgtcttatcatgtgggagcataagttatgtggtcaaaatagaaggattttgaactaaagttgattcaggttgtcttgcaccgtttcaaagtgctgccagttgaaaagggaagcattatgtttacaaatctgttttaaaaagtttgccaaaattttggtagtatctttaataaagatgtttgtcttcagcatccaaaaaagtgaa

SEQ ID NO: 42 - protein, 1003 aa, Rattus norvegicus
MAAGGAAGLREEQRYGLACGRLGQDNITVLHVKLTETAIRALETYQSQKWGAGSGCCGGTRLGEGGQPESPGRGPGPLDRGCGRAAPGGGVRLAGAARPLRDFLTAPAGFLACLVPRRDRVEEREVRRGRAQLPGLGDTGTPSVGVSVHGVPGKAGTLALRGGGGGHTLHSQGLCALGSRRLAAYHVATNRHRISGRDPEDSKTTRAWNKTAGSGERNSQKLGVAERRAVSGADSLSVGEERARRMEHESMEQSGVDVSMNTHLKAVTMTLKNREPVQLETLGIRGSNIRYFILPDSLPLDTLLVDVEPKVKSKKREAVAGRGRGREFNSFSTFNSVPRTPRAVNESDSQEDIREVLLELNLKGCQDLVGEKRGKYVLGLRFHSMRKGQTVCLQRGEWELTFPLDSEQVLACLTQVSESSVFGYKDVQLMKIPKNDPFNEVHNFNFYLSNVGKDNPQGSFDCIQQTLSSSGASQLNCLGFIQDKITVCATNDSYQMTRERMTQAEEESRNRSTKVIKPGGPYVGKRVQIRKAPQAISDAVPERKRSTPMNPANTIRKMHGGSSVSQRPYRDRVIHLLALKAYKKPELLARLQKDGVNQKDKNSLGAILQQVANLNPKDLSYTLKDYVFKELQRDWPGYSETDKQTLDLVLSRKLNPSQNAGTSRPESPMCSSKDAASSPQKRPLDSDFIDPLTNKKARISHLTNRVPPTLNGHLNPTNEKSCASLLPPPAAAAIPTPSPLPSTHLPVSNPTQTVNSNSNSPSTPEGRGTQDLPVDSFSQNSSIFEDQQDKYTSRTCLEETLPSSSALLKCPKPMEEEHPVSHKKSKKKSKKHKEKDQIKKHDIESREEKEKDLQREETAKLSNDSPNPNEGVKEECTASMEPSSTIELPDYLIKYIAIVSYEQRQNYKDDFNAEYDEYRALHARMETVARRFIKLDAQRKRLSPGSKEYQNVHEEVLQEYQKIKQSSPNYHEEKYRCEYLHNKLAHIKRLIGEFDQQQAESWH

SEQ ID NO: 43 - DNA, 1904 bp, Rattus norvegicus
  CDS (62)..(1078)
gagcgactgcgggcccaggttgtcgcgctgcgctcggtgagtacaggatccagcttccaggatgtggtggtccttgatccctctctcttgtctgctggcactgaccagtgcccatgacaagccttcctcacacccactgtcggacgacatgattaactatatcaacaaacagaatacaacatggcaggctggacgcaacttctacaatgttgacataagctatctgaagaagctgtgtggaactgtcctgggtggacccaacctgccggaaagggttgggttcagcgaggacataaatctacctgaatcctttgatgcacgggaacagtggtccaattgcccgaccatcgcacagatcagagaccaggggtcctgtggctcttgttgggcatttggggcagtggaagccatgtctgaccgaatctgcattcacaccaatggccgagtcaatgtggaggtgtctgctgaggacctgcttacctgctgtggtatccagtgtggggatggctgtaatggtggctatccctctggagcatggaacttctggactagaaaaggcctggtttctggtggagtatacaattctcatataggctgcttaccctacaccatccctccctgtgaacaccatgtcaatggctcccgtcccccatgcactggagagggagatactcccaagtgcaacaagatgtgtgaggctggctactccacatcctacaaggaagataagcactatgggtacacttcctacagtgtgtctgacagcgagaaggagatcatggcggaaatctacaaaaatggcccagtggagggtgcttttactgtgttttctgacttcttgacttacaaatcaggcgtatacaagcatgaagccggtgatgtgatgggaggccatgccatccgcattctgggctggggaatagagaatggagtaccctactggctggtagcaaactcctggaacgttgactggggtgataatggtttctttaaaatcctcagaggagagaaccactgtggaattgaatcagaaatcgtggctggaatcccacgcactcagcagtactggggaagattctaatctgctgggacttgattgtatagtccttaggagactttttccaaaatttagcaatgcaggcagggagtgaggcagataggagtctttgattctttgagttcacctaacatgcatgaagctttcagcaaggtttgggtgactggaccagagctgccccccatcagagccaccctacctggctcccttctagcctatcccatgacagtgatcacagcctacatcctagcctctccctagactagtgctgtgtgtagcgcctgctgctgtggctctgtctgccatccctcccacccccacctcagcatccccagaccaagggctttggctgcaggatttccaaaggaatgaacaccaactacatgatgagaggacagatgccacccgccagctgcaccttgaagctagtcacttctaggactgtggcaggctttgatggcccaatgcagttctctggagaaaactacctttccccaaggcatctgcacccattgacaatggtaatgtgcccatctctccttggtcctgccctcaaccgatgcttttccagtcagggttttgttttttgttttgttttgtgtacctcaactgagttatgaagatttgtactggttttacagatcatctcatcgtatggaaaagaacaagcttcgtggtcagtttactgggtgaccggcagacaccacaatcaaactagtctggggaaaacctgcttttttgttgtaggtgccacgtaaccctgtcagtttgacaaggaatgaccgtgccaataaaccaattctccctctgcttggaaaaaaaaaaaaaaaaaaaaaaaaaaaaaaaa

SEQ ID NO: 44 - protein, 339 aa, Rattus norvegicus
MWWSLIPLSCLLALTSAHDKPSSHPLSDDMINYINKQNTTWQAGRNFYNVDISYLKKLCGTVLGGPNLPERVGFSEDINLPESFDAREQWSNCPTIAQIRDQGSCGSCWAFGAVEAMSDRICIHTNGRVNVEVSAEDLLTCCGIQCGDGCNGGYPSGAWNFWTRKGLVSGGVYNSHIGCLPYTIPPCEHHVNGSRPPCTGEGDTPKCNKMCEAGYSTSYKEDKHYGYTSYSVSDSEKEIMAEIYKNGPVEGAFTVFSDFLTYKSGVYKHEAGDVMGGHAIRILGWGIENGVPYWLVANSWNVDWGDNGFFKILRGENHCGIESEIVAGIPRTQQYWGRF

SEQ ID NO: 45 - DNA, 3247 bp, Rattus norvegicus
  CDS (36)..(2891)
gtcgctgccgcctccgctaccgctagtggaagaagatggcggaaggcggagcggcggacctggacacccagcgttctgacatcgcgacgctgctcaaaacctcgctccggaaaggggacacctggtatctagtagatagtcggtggttcaaacagtggaaaaaatatgttggctttgacagttgggacaaataccagatgggagatcaaaatgtctatcctggacccatcgataactctggacttctcaaagatggcgatgctcagtcacttaaggaacaccttatcgacgagttggattacatactgttgccgactgagggctggaataaacttgtcagctggtacacactgatggaaggccaagagccaatagcaagaaaggtggtagaacagggcatgtttgtaaagcactgcaaagtggaagtctacctcacagaactgaagctctgcgagaatgggaacatgaacaatgttgtaactcggagatttagcaaagctgacacaatagacacaattgaaaaggaaataagaaagatcttcaatattccagatgaaaaggaggccagactgtggaacaagtatatgagtaacacatttgagccactgaataagccagacagcaccatccaggatgccggcctataccaaggacaggtgttagtgatagaacaaaaaaatgaagacggaacgtggcccaggggtccctctgctcctaatgtgaaaaactccaattattgtctcccgtcatatactgcttacaagaactatgattattcggaacctggaagaaacaatgaacagccaggcctgtgtggcctaagtaacttgggaaacacatgtttcatgaactcagccattcagtgtttgagcaacacacctccacttactgaatatttcctcaatgataagtaccaagaagagctgaattttgacaatcccttaggaatgagaggtgaaatagctaaatcatatgctgaactcatcaagcagatgtggtctggaaagtttagctatgtcactccaagagcatttaagacacaagtaggacgttttgcacctcagttctctggatatcaacagcaagactgccaagaactactagctttcctattagatggattacatgaggatttgaatagaattaggaaaaaaccatatatccagttaaaagatgctgatgggaggccagacaaggtggttgctgaagaagcctgggaaaaccatttaaaaagaaatgattctatcatagtagatatatttcatggccttttcaagtctacgttagtgtgtcctgagtgtgctaagatctcagtgaccttcgaccctttctgttacttgacacttccattgcctatgaaaaaggaacgtagcttagaagtttatttagttagaatggatccacttgctaaacctatgcaatacaaagtcattgtgcccaaaattggaaatatactagacctttgtacagcactgtctgctttgtcgggagtacctgcagataagatgatagtcactgacatatataatcatagatttcacaggatatttgcaatggatgagaacctgagtagtattatggagcgggatgatatttatgtgtttgaaattaacatcaacaggacagaagacacagaacatgtggttattcctgtttgcctaagagaaaagttcagacactcaagttacactcaccacactggctcttcactgttcggccagccttttcttatggccgtgccacggaacaatactgaagataaactctacaatctcctgctcttgagaatgtgccggtatgtcaagatgtctactgaaactgaggaaactgatggacccctgcgttgctgtgaggaccagaatattaatgggaatggtccaaatggcatacatgaagaaggctcaccaagtgagatggagacagatgaaccagatgatgagtccagccaggatcaagagcttccctcagaaaatgagaacagtcagtctgaagattcagtcggaggagataacgattctgaaaatggattgtgtactgaagaaacatgcaaaggtcgactgacgggacacaaaaagcgattgtttacattccagttcaacaacttaggcaacactgatatcaactatatcaaagacgacaccaggcatataagatttgatgataggcagcttcggctagatgaaagatcttttctcgccttggactgggaccctgacttgaagaagagatattttgatgagaatgctgcagaggattttgaaaaacatgaaagcgtggaatataaacctccaaaaagaccctttgtgaagttaaaagactgcattgaacttttcacaacaaaagagaagctaggtgctgaagacccctggtattgtccaaattgtaaagagcatcagcaagccaccaagaaattagatctgtggtccctgcctcccgttcttgtggtgcatctcaagagattctcctatagtcgctacatgagagacaagctagatacattagtcgattttcccatcagtgacttggatatgtcggaattcctaattaacccaaatgcaggcccttgccgctataatttgattgctgtttccaatcactatggagggatgggaggaggacattatactgcttttgcaaagaacaaagatgatggaaagtggtactattttgatgacagtagcgtctccagtgcatctgaagaccaaattgtgtccaaagcagcatacgtgctcttctaccagagacaggacactttcagtggaactggcttcttccctctcgaccgagaaactaaaggtgcttcagctgccacaggtgtccccctggaaagtgacgaagacagcaatgataatgacaatgatctagaaaatgaaaactgtatgcacactaattaatgaaagtccaagaagccatacgagagagagagaaagagagagagagactttcctgccggtggtatctgtggagacggagttacccaccgcattaaacagaggtttgagaggaggcgtttcagatacccgaatgtaaatcctttatcagattttaaattctgcagtacttgccgtgaaacacaatgaaaactttaacagaaattgttttttaaaacatttacagttttgtatttacaagctaaatatatataggaaatcacaaataaatcccttttaagttaaaaaaaaaaaaaaaaaaaaaaaaaaaaaaaaaaaaaaaaaaaaaaaaaaaaaaaaaaaaaaaaaaaaaaaaa

SEQ ID NO: 46 - protein, 952 aa, Rattus norvegicus
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFNIPDEKEARLWNKYMSNTFEPLNKPDSTIQDAGLYQGQVLVIEQKNEDGTWPRGPSAPNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERSLEVYLVRMDPLAKPMQYKVIVPKIGNILDLCTALSALSGVPADKMIVTDIYNHRFHRIFAMDENLSSIMERDDIYVFEININRTEDTEHVVIPVCLREKFRHSSYTHHTGSSLFGQPFLMAVPRNNTEDKLYNLLLLRMCRYVKMSTETEETDGPLRCCEDQNINGNGPNGIHEEGSPSEMETDEPDDESSQDQELPSENENSQSEDSVGGDNDSENGLCTEETCKGRLTGHKKRLFTFQFNNLGNTDINYIKDDTRHIRFDDRQLRLDERSFLALDWDPDLKKRYFDENAAEDFEKHESVEYKPPKRPFVKLKDCIELFTTKEKLGAEDPWYCPNCKEHQQATKKLDLWSLPPVLVVHLKRFSYSRYMRDKLDTLVDFPISDLDMSEFLINPNAGPCRYNLIAVSNHYGGMGGGHYTAFAKNKDDGKWYYFDDSSVSSASEDQIVSKAAYVLFYQRQDTFSGTGFFPLDRETKGASAATGVPLESDEDSNDNDNDLENENCMHTN

SEQ ID NO: 47 - DNA, 1458 bp, Rattus norvegicus
  CDS (91)..(1074)
ttaccagcaagtgtgcatagggacaaacggagaccgatcgcagcaggctgagggtgcaataacgacctcctggaccgacccgcccgcgccatgcgctcgcacactggcttgagggcgcttgtggcgcctggttgctcgctcctccttctatatcttctggcggcgacgcgcccggatcgggctgtgggcgatcccgcagactcagcgtttacaagtctacctgttcgagaggaaatgatggctaagtacgcaaatctgtccttggagacctataatatctcactgacagaacagaccagagtatcagaacaaaacataactttggaaagaccttctcacctggaactcgaatgcacgttcacggcaactgaggatgtgatgtcaatgaatgtgacttggaagaaagatgacgcgctccttgagactactgatggtttcaatacaactaaaatgggggacaccttatacagtcaatacaggttcaccgtttttaatagcaaacaaatgggaaaatactcttgtttccttggagaagagctcagaggaacctttaatatcagagtacccaaagttcatgggaaaaacaagcctttgatcacttacgtgggggattctactgtgctgaagtgtgaatgtcaaaattgtcttcctttaaattggacttggtacatgagtaatggaactgcacaggttcccatcgatgttcacgtgaatgataagtttgatataaatggttcctatgctaacgaaacaaagctcaaggtaaagcaccttttggaggaagatggaggatcctactggtgccgggcagccttcccgttaggggagagtgaggaacacattaagctggttgtgctgagtttcatggtgcctctcaagccattccttgccataattgctgaagtcatcctcttagtggccatcatcctgctttgtgaagtgtacacgcagaagaaaaagaatgacccagatgatgggaaagaatttgaacaaatcgaacagttgaaatcagatgatagcaatggaatagaaaacaatgtcccccggtacagaaaaacggactctggggatcagtgaatccaaaaagaactttttgccttgggaagacgtacagcactggagtttgaatttcagcattcattggagaacatccgagttttagatttttccgaagacaaagatgccctcactgtgcccaggagtagtttcgtaaacaatatagaccatctcctacctaaagatgtattttcattgtgtaattgcttttcattaaagcaaggtacattgtttcagctgtgttctgtgagctgtaacctagataataaagtttaccctctctgggagatcagggggacagggcagacaccagcaaaatcagttaataacccacaaagtaaatattgctaaagatctgtttattaccaaagaatttattaaagagaaagtgtttgccaaaaa

SEQ ID NO: 48 - protein, 328 aa, Rattus norvegicus
MRSHTGLRALVAPGCSLLLLYLLAATRPDRAVGDPADSAFTSLPVREEMMAKYANLSLETYNISLTEQTRVSEQNITLERPSHLELECTFTATEDVMSMNVTWKKDDALLETTDGFNTTKMGDTLYSQYRFTVFNSKQMGKYSCFLGEELRGTFNIRVPKVHGKNKPLITYVGDSTVLKCECQNCLPLNWTWYMSNGTAQVPIDVHVNDKFDINGSYANETKLKVKHLLEEDGGSYWCRAAFPLGESEEHIKLVVLSFMVPLKPFLAIIAEVILLVAIILLCEVYTQKKKNDPDDGKEFEQIEQLKSDDSNGIENNVPRYRKTDSGDQ

SEQ ID NO: 49 - DNA, 4157 bp, Rattus norvegicus
  CDS (2884)..(4002)
acgagccgcattgcaggtgcggggcagcgccccggggtccggccccacgccggcgggatgctgcgggcctcagctccccgcccgcgtcacacctgccgcctcccggacccgcagccctggcgaccgggtggctccgcgtcacctggatgtcagcatggcagccacaaacctggagaaccagctgcacagtgcgcagaagaacctgctcttcctccagcgggaacatgccagcacactcaaggggctgcacgctgagatccggcggctgcagcagcactgcacagatctaacatatgagctgacactcaaaagtttcgaactgacagagctggcactccagagacgctgaccccagctgacgctcaagctgttttaacttcagcaccttcagattctggagacgttcttttacgcagagacggctcttccagaacgacggagctgaagaggcgctgtgaagagctggaagcccagctgaaggccaaagaggaggaaaaccgcaagctgcttcaagaactggagcagaagaatgccgccattgctgtgctggagaacacggtccgcgagagagagaagaagtacctggaggagctgaagatgaagagccacaagctgagcatgctgtcgggcgagctggagcagcgcgccagcaccgtggcctacctcacctcacagctgcatgcggccaagaagaagttgctgagctccagtggcacctcggatgccagccccgccggcagccctgcactggccagctacaagcccacaccccccaaggacaagctgcctgagacaccccgccgccgcatgaagaagagcctgtcagccccactgcaccctgagtttgaggaggtctacaggtttggagctgagagccgaaaactcctccttcgagagcctgtggatgccatgcctgaccccaccccattcctgctggcccgggagtctgctgaggtccagctcaaggagcggcctcttgtcatccctcccatcgcctcagatcgtggcaccagtgggcagcatagcccagcccgagacaagccacacaagacccacattggggtggctcaccgcattcaccatgccactccatcacaggcccagcccgagggagagacgagggccgtggaccaggtgaatgcagggaaggtggtgcgaaagcactcagggacagaccggactgtgtgaagcctgcggcctgtcaccgcagccatccacgcactgtgagcgccacagggaacctcgcccacatcttcctcgcctacccatgccaaatgtttcctcctgttgaagcctggctcctggctccatagtgacagtgctacaatggtgtccccgctgaagatatttactggcactgtggccaatgcctgcatgccaatagcttgcttccagccccacgccatgagatctcggcacactcttatatggccatggctgaatcactacgccacacagaatcagaatcgcctgaagtcgaagcaaaaataaggagctctctctctctttccctctctctctctttctctccctccccagttccagggcacctacgagagcagagcagcagcttctgcttccccctggagactcctgccttgcacagtccctttacaagcagctatgtctcatgatcttagcaagatgacctcaaccctgatgtcgccccaacctgaatgtgtgttcataatttttttttgttagtttcatccagatgttcaggatccaacaaatgttattttctaaacccgccctccttttcctctgcgtctttttgaagcaccaagggactggaggaggtggctggggaggaccaagcaggtggggtgggagtacttgctgcccagagctgttgctgagatggatgtctgcgtttctggcagaagctgcattccgtcctgcccaggcttcccactgcctgcctgtctccagctggccagcccagtctcacagtgtaagaggcacagccagtggctgtttgtcctgaagccacacacagccctggctgagaagaggtctggagctcgggcaggaactgaatgcttgagccttcagtgctcttgccaagaaagggggtgtgtgctggagcagacgagtggaaccccaccagtggcgtagcaggaagggagagagaacgcacttgccttaaaacctgttctggcctgtgctctgagaagctgcagccaattccagcacccgcgtggcagctcacaattagctgtaactccaaggcccaccttacatgtggtacacggatattcaggcaggctctcacacatacacagaaataaaatcttaaaaaaataaaaaaaagcggggccgggcggaggcagcagcagcacgctgtgcctcccgccgtcgctccactctcgccgtccggcctctgtcctcgccggggtatggccccttgggagaggccttgagatctgcgggggtccgagagtcggggcgccgactttccggttattttagtgggaagggctgcttctcaaagtggattaccccaactcctccgggggcgggaagcggggatcctcccccaaccgcaaatactcaaagaaagcaccggctgaagacatagacgatggagagtctcggttctcagagtccccttctaataccttcggcttcgttgcctactctgtgaacaacggggagaagtcgagggctaaggttaaatcccaccagcctacttattccagcacctcccactccgaacgatctgggtcccccatcccatcgccctcacccagaaagcagcgttgcttagaccagcgagaaatggagctggaggagaggaacgaacactactctaaaaagaggaggattagagagtacaacctcaatgtatttgattcacattggctgggaacaccttccaccatggccacctcagcaagttcccacttgaacaaaggcatcaagcagatgtacatgaacctgccccagggcgagaagatccaactcatgtatatctgggttgatggtaccggggaagggctacgctgcaagacccgtactctggactgtgaccccaagtgtgtagaagagttacccgagtggaactttgatggttctagtacgtttcagtctgaaggctccaacagcgacatgtacctccatcctgtggccatgtttcgagaccccttccgcagagaccccaacaagctggtgttctgcgaagtattcaagtataaccggaagcccgcagagaccaacctgaggcacagctgtaagcgtataatggacatggtgagcagccagcacccctggtttggaatggaacaggagtatactctcatgggaacagacggccaccctttcggctggccttctaatggcttccctggaccccaaggaccctattactgcggtgtgggagctgacaaggcttatggccgagatatcgtggaggctcactaccgggcctgcttgtatgctggaatcaagatcacagggacaaatgccgaggttatgcctgcccagtgggaattccagataggaccctgcgaagggatccgcatgggagatcatctctgggtagcccgttttatcttgcatcgggtatgcgaagactttggggtgatagcaacctttgaccccaagcccattccagggaactggaatggggcaggctgccacaccaactttagcaccaaggccatgcgggaggagaatggtctgaggtgcattgaggaggccattgataaactgagcaagaggcaccagtaccacatccgtgcctacgaccccaaggggggcctggacaacgcccgccgtctgactggattccacgaaacctccaacatcaacgacttttccgctggcgttgccaaccgcagcgccagtatccgcattccccggattgtcggccaggagaagaagggttactttgaagaccgtcggccttctgccaattgcgacccctatgcggtgacggaagccatcgtccgcacgtgtctcctcaacgaaactggcgacgagcccttccaatacaagaactaagcggactcgacttccagtgatcttgagcccttcctagttcaccccactcccaactgttccctctcccactggtccccactgtaactcaaaaggatggaataccaaggtctttttattccttgaaaaaaaaaaaaaaaaaaaaaaaaaaaaaa

SEQ ID NO: 50 - protein, 373 aa, Rattus norvegicus
MATSASSHLNKGIKQMYMNLPQGEKIQLMYIWVDGTGEGLRCKTRTLDCDPKCVEELPEWNFDGSSTFQSEGSNSDMYLHPVAMFRDPFRRDPNKLVFCEVFKYNRKPAETNLRHSCKRIMDMVSSQHPWFGMEQEYTLMGTDGHPFGWPSNGFPGPQGPYYCGVGADKAYGRDIVEAHYRACLYAGIKITGTNAEVMPAQWEFQIGPCEGIRMGDHLWVARFILHRVCEDFGVIATFDPKPIPGNWNGAGCHTNFSTKAMREENGLRCIEEAIDKLSKRHQYHIRAYDPKGGLDNARRLTGFHETSNINDFSAGVANRSASIRIPRIVGQEKKGYFEDRRPSANCDPYAVTEAIVRTCLLNETGDEPFQYKN

SEQ ID NO: 51 - DNA, 1869 bp, Rattus norvegicus
  CDS (6)..(1406)
ccaccatgtggggatgtccgctagggctgctattgctgctgctggctggccaggctgccctggaggcccggcggagtcgttggcgcagggagctggcgccagggctgcacctgcggggcatccgggacgccggtggcagatactgccaagagcaggacatgtgctgccgcggccgtgctgacgagtgtgctttgccctacctgggagccacctgttactgtgacctcttctgcaaccgcaccgtctctgactgctgccctgacttttgggacttctgcctcgggattccaccccccttccctcctgtccaaggttgcatgcatgcgggccggatctacccaatcttcggaacctattgggaaaactgcaatcggtgcacctgccatgagaaagggcagtgggaatgtgaccaggagccatgtctagtggacccagccatgattaaagccatcaaccggggcaactacgggtggcaggctgggaaccacagtgccttctggggcatgaccctggatgagggcattcgataccggctgggcacaatccgcccatcttcctctgtcatgaatatgaatgaaatttatacagtgctgggacaaggagaagtgctacccactgcctttgaggcttccgagaagtggcccaacctgatccatgagccgctggaccagggcaactgtgcgggttcctgggctttctccacagcagctgtcgcatctgaccgagtgtccatccattctttgggacacatgacacccatcctctcaccgcagaacctgctatcctgtgatacccaccaccagaagggctgccgaggtgggcgtctcgatggtgcttggtggttcctgcggcgccgaggggtggtgtctgataactgttacccattctccggccgtgagcagaacgacgaggccagccccactcctcgatgcatgatgcacagccgcgccatggggcggggcaagcgccaggccacttctcgctgccccaatagtcaggttgattccaatgacatctaccaggtcacgcctgtctaccgcctggcctccgatgagaaggagatcatgaaggagctaatggaaaatggccccgttcaagcgcttatggaagtacacgaagacttcttcttgtaccagcgaggcatctacagccacacacctgtaagccaggggaggccagagcagtaccgccgacacgggactcactctgtcaagatcacagggtggggagaagagacactgccagacggaaggacgatcaagtactggactgctgccaactcgtggggcccatggtggggtgagaggggccacttccgaatcgtgcgtggcatcaacgagtgtgacatcgagaccttcgtgctgggcgtctggggccgcgtaggaatggaggacatggggcaccactgagtctcggccactaagctaggtgggatccacagccacggaagaggccttgggggccacgccgatgaggccttgggaccaggtgctaatcccctcagactcagatccacacaaacgcagaaccccacctgggagctaatgtccctcagggagcaacagtgaggctggagggaaccctcagacatcacagccggaactggaaagggcccggcttggaaactgcagggagtaaaattcccaggcccctggtcagccagcccaagaccatgggagctaagacaccccaacctctttatcctcctacccaacctcattcttattttttcattctctttggtggattctgcccatccccctggcctccgttctggctggacctttccatcctcaacactgctttcttactctttaaaaatatttatttttcttttcattaaaataaaaccaaagtattgataattgc

SEQ ID NO: 52 - protein, 467 aa, Rattus norvegicus
MWGCPLGLLLLLLAGQAALEARRSRWRRELAPGLHLRGIRDAGGRYCQEQDMCCRGRADECALPYLGATCYCDLFCNRTVSDCCPDFWDFCLGIPPPFPPVQGCMHAGRIYPIFGTYWENCNRCTCHEKGQWECDQEPCLVDPAMIKAINRGNYGWQAGNHSAFWGMTLDEGIRYRLGTIRPSSSVMNMNEIYTVLGQGEVLPTAFEASEKWPNLIHEPLDQGNCAGSWAFSTAAVASDRVSIHSLGHMTPILSPQNLLSCDTHHQKGCRGGRLDGAWWFLRRRGVVSDNCYPFSGREQNDEASPTPRCMMHSRAMGRGKRQATSRCPNSQVDSNDIYQVTPVYRLASDEKEIMKELMENGPVQALMEVHEDFFLYQRGIYSHTPVSQGRPEQYRRHGTHSVKITGWGEETLPDGRTIKYWTAANSWGPWWGERGHFRIVRGINECDIETFVLGVWGRVGMEDMGHH

SEQ ID NO: 53 - DNA, 3628 bp, Rattus norvegicus
  CDS (6)..(1085)
tggggatgctagtgcctactgcgctggctgctcggctgctgagcctgttccagcaacagctgggttccctctggagtggcctggccatgctgttctgctggctgagaatagcattggggtggccagatcctggaaagggacagccacgggtccggggtgagcccaaggagactcaggagacccacgaagatccaggcagtgctcagcccacaacccccgttagtgtcaactaccacttcacgcgtcagtgcaactacaaatgtggcttctgcttccacacggccaagacatccttcgtgctgcccctggaggaggccaagcgaggactgcttctgctcaaacaggctggtatggagaagatcaacttttcaggaggagaacccttcctacaggacaggggtgaatacttgggcaagctcgtgagattctgcaaggaggagctagccctgccctctgtgagcatagtgagcaatggcagccttatccgagagagatggttcaaggactatggagactatttggacattcttgctatctcctgtgacagctttgatgagcaggttaatgttctcattggtcgtggccaagggaaaaagaaccatgtggaaaaccttcaaaagctgcggaagtggtgcagggattacaaggtggctttcaagatcaattccgtcattaatcgcttcaacgtggacgaagatatgaatgagcacatcaaggcgctgagccctgtccgctggaaggttttccaatgcctcctgattgagggtgagaactcaggagaagatgccctgagggaagcagaaagatttcttataagcaatgaagaatttgaagcattcttacagcgtcacaaagatgtgtcctgcttggtgcctgaatctaaccagaagatgaaagactcctaccttatcctggatgaatatatgcgctttttgaactgtaccggtggccggaaggacccttccaggtccatcctggatgttggcgtggaggaagcgatcaaattcagtgggtttgatgagaagatgtttctgaagcgcggggggaagtatgtatggagtaaggctgacctgaagctagactggtgaggtgagattggacggagactctaaccagctacgaggagtccacgagcagccgctcgccaccacgcacatctggctgtctacggactgttgctactgagaatgcactctatgttctttctttcagtttgactgaactaagaaaggagtaaaaccattctgtgaacccttatacagccgataagggtcctccacaggggaaggagttcagttcacagaggaacaaggtccagcgatctcaaagaactcacattttatttgtattcgaatttttgtatggatcgttactgtgaatcccatgtttccttttccctttctgttcccccatgagaaactgggttattttctgctaactatgccagttttgtcaacaggaaggatcacaaaacacaccctgcgattactgctgactgttctaatgcttttctgctttggaatctcggactttttgctcagaaatatagaatcaagaagtgtgttatagagccgtacgtgagaaccttactcttttgcaaggagcatatggaaggaggtacttctgaggacggtgtgttcatggcctcagcctctgctgctgctgctgcttcaaggatggctgttctatatcgtaccaattccttcaaggttctggacaaagccaaggtacactttacctttcatggcatggccgtgaagatggcactgacagccgcaaccaccccagcactgaggctgcctgagtctgtcaggctaaaaggttgtcccacttttctgaaaagctacaagggctcttctgcacatccagcagaatcatgacaggatgggcatggaggactcttcaggagtatgaaatgtggctgctgctccttaccatatttctcatgaaactctgaaataagacctagtagtctaggtcaccatgagaaaataatttcttctctacatcaaataagcaaaagagagaacatgaactagcccaaatcttcttcctgtggaagcattgcatgagtttattcaacctagacagcctcactgcttggaaaataattccctggaaattaaataaataccatggaaaagaaattaaactcctctgcaatcccactgtggccactagagggcaccaggaagtcagacttcattgtctcagagacttttcttcagtctttttttagaatttggtgaattttaattctgttccttatctgttttgtctttgttttttgttttggtgcctattactaaaattaatactaaacattttccaaggaactggaattttggccatttacaaaaggtcttcattctttagtgaggtcactataattcaactaagacatactctgtctcattaaaaataggtcaggttttcctgtgttttctgtcttgtttggttgtgtgtgtgtgtttacctatttttgcttgaatatagatataactatgtatcccaggctagcctggaactcatgatacctctgccttaacctccccctcttaagtgcccaggaattacccaactcctgtacagagatttttaagagaagtttgagtcacattcccccctgcagattgtgatttttttttttaagttaagtcctgtgtacatagttcacatgggaaaccatgacatcccagaggacatgggcagacacccacttggtcacacgacagagggaagctgcatgtttctttccctaattaaagtagagcatgaagaaagtatgattggaaagtgagccatcatttacatgtacccccaaccccttgtgcatgcactggcggtgggagatctctgtgcttaaaatattgaaaagtcccctttccttaaagggaaaaattacgatattttactatcgtagtacaaatcaaacttggattagatcatacattggtttgctttttcagtgttaaatktgggtgttcattttgtaaagcatacgctccctgctggggaaacaaggagtgatctagttgacatttgttaagtgtgaccagcagataccttgtgtcccatgattgatttatgcagagcagtaatactgggaagggggatttacttaagaacatcgacgggtaggacatccaacactgatttcaaagttttggcttctttattataatgtgttcggcggtaaaaaaaagtcatgggttgttttctttgaagctgaaagtctcatggtgttatttacctaaattatctgaaaaatttaaaaggggcccaggtttcattctgcgttggagtttgggggtctaattgttgcataaataaacaggcaaccaatcatcagaggttgactcctcccatcaataattcagctcgtgttttacccttttgatggactgatgttgtttcataagctattagtagtccaagaacaatcatgcaggcctcatgtgaatgtgttcagatgaggtttgatgttcgggatacatgtgcatgtatctccctgtagaatgacactgtattttgaggcattgctctagtgtatggaaacgtgatctgtgtattaaaatgttttatgcatgacctcaaaaaaaaaaaaaaaaaaaaaaaaaaaaaaaga

SEQ ID NO: 54 - protein, 360 aa, Rattus norvegicus
MLVPTALAARLLSLFQQQLGSLWSGLAMLFCWLRIALGWPDPGKGQPRVRGEPKETQETHEDPGSAQPTTPVSVNYHFTRQCNYKCGFCFHTAKTSFVLPLEEAKRGLLLLKQAGMEKINFSGGEPFLQDRGEYLGKLVRFCKEELALPSVSIVSNGSLIRERWFKDYGDYLDILAISCDSFDEQVNVLIGRGQGKKNHVENLQKLRKWCRDYKVAFKINSVINRFNVDEDMNEHIKALSPVRWKVFQCLLIEGENSGEDALREAERFLISNEEFEAFLQRHKDVSCLVPESNQKMKDSYLILDEYMRFLNCTGGRKDPSRSILDVGVEEAIKFSGFDEKMFLKRGGKYVWSKADLKLDW

SEQ ID NO: 55 - DNA, 1146 bp, Rattus norvegicus
  CDS (456)..(1001)
ggcgggcggcggccggggcggcccaggggctgccgcggggactcggggcgccgccgagcggccaggcgctggggcgggccgagcgcaccgcggggcccggacacggcgggttcgaggccgggcccccgcgcggggagctgcggggtggagactttgccgcgcggcgcggagcaggcgggagccggcgggggcctcggggagccccagggcggccgggcccggatcccgggggagcctcgcgagtccgggccggaacctcggtgtgcgcatcccctccggggcccgggctgcaggagcacagagcaaccgggcctcgcggagctcagagcgcgcatgacccggggcgcaccgtgggacccgggcgcggggctggcggagagctgccccgcggggctagcagccgcggagtggcctacaggggccctcccccggaggggccgggtcggggcggggagatgaacggcttcagcacggaggaggacagccgcgaagggccccctgccgcccccgccgccgccccgggctacggccagagctgctgcctcatcgcggacggcgagcgctgcgtccggcccgcgggcaacgcctccttcagcaagagggtgcagaaaagcatctcgcagaagaaactcaagctggacatagacaagagcgtaaggcacctgtacatctgcgacttccacaaaaatttcatccagagcgtccgaaataaaaggaagaggaagacaagtgatgatggcggagattcccctgagcatgatgccgacattcctgaggttgatctgttccagctacaggtgaacactctccgacgctacaaacggcactacaagctgcagaccagacccggcttcaacaaggcccagctagcagagactgtgagccgacacttcaggaacataccagtgaatgagaaagagacgcttgcctatttcatctacatggtgaagagtaacaggagcagactggaccagaagccagagggcagcaagcagcttgagtgaggtgcagccgcccagtgaggtggaagccctgatgagaggacagccatgcactgcttgatgacaggtggcccctgcaacgtctcagccaataaggactgttcgtatctactgtaaataaagtttgaatgatggacactgtaaa

SEQ ID NO: 56 - protein, 182 aa, Rattus norvegicus
MNGFSTEEDSREGPPAAPAAAPGYGQSCCLIADGERCVRPAGNASFSKRVQKSISQKKLKLDIDKSVRHLYICDFHKNFIQSVRNKRKRKTSDDGGDSPEHDADIPEVDLFQLQVNTLRRYKRHYKLQTRPGFNKAQLAETVSRHFRNIPVNEKETLAYFIYMVKSNRSRLDQKPEGSKQLE

SEQ ID NO: 57 - DNA, 854 bp, Rattus norvegicus
  CDS (402)..(740)
ctggtttgaacctgaacaggaggagcctgtgggaaaaggggcggaactttatgggggaccgatatctttgcaaattagctggaggtgtggggttggggtgtcgaagaggcgatcaacttggaaagtgggccaatccgtgacgcaaatctttcagtcccaccctcaaccccgcctcccactgctccgctcctccgctcctaattggttctagtttctccgcccgccagacttcttctccattcagcagaagttttacaaaggcgtctgtaagggaactaggagctaaaatgaaaatagctctgattgggcacctttggaccttatttgcatgggcaattgggattggttggcttcttgggtggccagggcggtgttggttccgcgcgtgaccacccacagctatggccggggtaggcgctgctttccgccgcctaggtgccttgtccggagctggggccttgagtttggccacctacggggcacacggcgcacagtttccggatgcctacgggaaggagctctttgacaaggccaacaaacaccactttttgcacagcctggccctgttaggggtaccctattgcagaaaacctgtctgggcagggttgctgctagcttctggaactaccttattctgcaccagcttttactaccaggctctgagtggagacactagcatccagactctggggcctgttggagggagcctgctgatcttgggctggcttgccttggctttttaaggtctcttgtttaagtgcccagtcctggcttttctgggtaattgggtgcagagggaaaggggaatgttgaagcagaaaagaaattaaaagaaaaaggcttacataaaaaaa

SEQ ID NO: 58 - protein, 113 aa, Rattus norvegicus
MAGVGAAFRRLGALSGAGALSLATYGAHGAQFPDAYGKELFDKANKHHFLHSLALLGVPYCRKPVWAGLLLASGTTLFCTSFYYQALSGDTSIQTLGPVGGSLLILGWLALAF

SEQ ID NO: 59 - DNA, 338 bp, Rattus norvegicus
tttttttttttttttataataatgcttttaattactgatttcaataacaagatccttactgtttagtgggttttgactgagacgtcgtttgctggatttctgttatgattaccatctctttctccaacatgatggttcatagttaacttttttttttttttttttggctgtttaagtcagtgtcttgattcccccacactctgttaaaagaaatcttatgtcatccaagagcatgaaacaggtaattaaactaagttgtacctctttggaataaaagatgtactggtttaaaatgggttgctgggtgtctatgtgtgggtgtgtgtatttccaatgca

SEQ ID NO: 60 - DNA, 675 bp, Rattus norvegicus
ttttttttttttttttttaaaatctgtgtactagctttattaagccacacaaaataaataaaaggaacgttcaatttaatacatttttattgagctcccacagggtgcatgaattaatgatttataatttacacaggtagaaaagtgatatgttagccccttctcttttgaaatgcaaactgcatcttctctttatagttcgagtgaattattcaatgtgagcaaggctcagtctgatgttgccataggtaaaagggacagtacaaacattttcccttctccaaaaaagggctttgtaccagagtaaaccagcctgttcatctaacaacagctggtcaaaaaggctagtgaatctggaagaaaagatgctctggaagcactgtcctgagaaggattcattccccagagcacagtaaaatgtagaagaaaattaaccaggtcaaaagctcactgagatccagaaagtaccagtcacagtttagaaggtgggaaagaagagagaaggaaaggaagggaaaggagagccaacactggctgagctgggcataactgacgttggtcgtgcgggtgttctgatctgggcagagagggtcaattcgtgccccatgaacagagtggtttaactgatcattctccagtgcaccaactgtattttcaagtataatttgtcatttg

SEQ ID NO: 61 - DNA, 548 bp, Rattus norvegicus
ttttttttttttttttttgcctgcttttgtttttactggaggctcaggtggcacatgacagttcataaagtggcttcagaggtgattgggggaggtcgaagggaaacaaaaaataaatgggtggggaagaaaaacaatcaggaggaggtatgagctggctggttccttctcagcctgacttaggggagggaggtgatgcctctgaacaataaggatggatcccttccttcaacccatgctaggggagaggggaaaaaaagagaaaaaagccaaaggctgttgctttggccctcctgagtctcaagggaaaagctgatgttttgatgtcatgaattatgggaaaggggggagggggatgctgggtaaagtcagttggaaactggcatataccagatggtagctctgggtgtcctctgagttggaatcactccagtatggtggtgggggggtccccactgttgataggtgctgaggtctccgcagctataggctgccgggggccatggggctgctgccgacgcctctggaagtatggacggtttcgtgga

SEQ ID NO: 62 - DNA, 472 bp, Rattus norvegicus
aaaaaaaaataataaatccagtttatccatacggcttcagaggtcacagtaaaatcagtgtagactgagtgaagggaagacctgcgtgtgtgcacctgtgtcatgtcattctgggaagagcagtccctccacgggggtgggggtggggcggggacgagtggcatgtggtgtgtgggggggtataggagactggtacacgaggacaaagggctaaactgatttcacgccgtttcccgttccctcggtccagaaagatcgtgtgaatttcttacaaatgagtttgaagtcaaaaatcaaaacaaaacgcaaacaaacggatcaaccatcccagccacggtaagcccttattcgtgtctgcacccgtgtgctctttggggctccttatccgctcccttctctcgccgcctttgtcctgattcccacagagatccatagtcgttagaggcaggactgaagactgtaggtcatgcgt

SEQ ID NO: 63 - DNA, 686 bp, Rattus norvegicus
gactaaattcacttttatttttttttaaatacataggaaacaggactctatcaaaagagaacaaagaagaaacgtcagccaagtgttgccaaaagagagcatgcacaagtctgcttcaggtctgtctccaagcaccggctgtacaggacctcagcacacaagtggagctgctggaagaggtgagccttactcaccaaacagaagaaaacaagaatgctaggaaacatcccccagaggtgcagtttccataggcatcgtcagtatttacgtctgaaggaattccaatgcgggctcgatgggaacaaaatgtatcttaaacagacattggagtataaaattgctgagccctatcagtcgttcagtttctcaaaaggatttgctatcaaaaagattagatttagtcacggacaccgagtgccttgatcagccgttccctggtgtaatcccaggggcagctctgactgtgcttcttggttttaaaaaggaacagaagaaattgaagttaaagccacaagtgatagaggagagaggaaatgtttggaccgtgtagtcgtcttgattgtaaacagcagacattgtgctttgtcgtcttatgtaaaacaaggacccaagcatggaagggtaatggatccagtgacaggaaaccagacacaagggtgctcaatgattggcagcagcatctgcat

SEQ ID NO: 64 - DNA, 1402 bp, Rattus norvegicus
  CDS (62)..(1099)
cgagcaaagcttctcttcagttccctggaagacaaggcaatacaaagatactttattaaaaatggcaatggtatcagaattcctcaagcaggcctgctatattgaaaagcaagagcaggaatatgttcaagctgtaaaatcctacaaaggtggtcctggatcagcagtgagcccctacccttccttcaatccgtcctcggatgttgctgccttgcacaaagctatcatggttaaaggtgtggatgaggcaaccatcattgacatccttaccaagagaaccaatgctcagcgccagcagatcaaggcagcatacttacaggagactgggaagcccctggatgaaaccttgaaaaaagcccttacgggccacctggaggaggttgttttggctatgctcaagaccccagctcagtttgatgcagatgaactccgtgctgccatgaagggacttggaacagatgaagacactctcattgagattttgacaacaagatctaaccagcaaatcagagagattactagagtctacagagaagagctgaaaagagatctggccaaagacatcacttcggacacatctggagactttcgtaatgccttgcttgctctcgccaagggtgatcgctgtgaggatatgagtgtgaatcaagatttggctgatacagatgccagggctttgtatgaagctggagaaaggagaaaggggacagacgtgaatgtgttcaatacaattttgaccacaagaagctatcctcatcttcggaaagtgtttcagaattatagaaagtacagtcaacatgacatgaacaaagccctggatctggaactgaagggtgacattgagaagtgcctcacaaccattgtgaagtgtgccaccagcactccagctttctttgctgagaaactgtatgaagccatgaagggtgctggaactcgccataagacattgatcaggattatggtctcccgttcggaaattgacatgaatgaaatcaaagtattttaccagaagaagtacggaatccctctctgccaagccatcctggatgaaaccaaaggagactatgaaaaaatcctggtggctctgtgtggaggaaactaaacatcccaactgctctgtaagattccgaggagaacatctcttagccgttgttttcttcctattgcaaggcttaagtaggaaagttgctttgtcagtaagtctaattaccttctttgaataatgtagcctataaatatgttttagatcattcatctgtacaatagagaaatacttgttttgttaattatgtttatcccaaattataaatccctgtaagcaagtcactttggtaccattcctgagaaagaagtttacatagaataaaataaaacaattttataagacaaaaaaaaaaaaaaa

SEQ ID NO: 65 - protein, 346 aa, Rattus norvegicus
MAMVSEFLKQACYIEKQEQEYVQAVKSYKGGPGSAVSPYPSFNPSSDVAALHKAIMVKGVDEATIIDILTKRTNAQRQQIKAAYLQETGKPLDETLKKALTGHLEEVVLAMLKTPAQFDADELRAAMKGLGTDEDTLIEILTTRSNQQIREITRVYREELKRDLAKDITSDTSGDFRNALLALAKGDRCEDMSVNQDLADTDARALYEAGERRKGTDVNVFNTILTTRSYPHLRKVFQNYRKYSQHDMNKALDLELKGDIEKCLTTIVKCATSTPAFFAEKLYEAMKGAGTRHKTLIRIMVSRSEIDMNEIKVFYQKKYGIPLCQAILDETKGDYEKILVALCGGN

SEQ ID NO: 66 - DNA, 2954 bp, Rattus norvegicus
  CDS (111)..(647)
ccggctccggagcaccgcgacttcgttcggtccgactccccggcggggtggcggcggccgggtccccacggtggcggccggagcagcggctgcaggagcccggctctaggatgaagttcaagcccaaccagacgcggacatacgaccgcgagggcttcaagaagcgggcggcctgcctgtgcttccgcagcgagcaggaggacgaggtgctgttggtgagcagcagtcggtacccagaccaatggatcgtgccgggaggaggggtggagccggaggaggagcctggcggtgctgctgcaagggaagtgtatgaagaggctggagtcaaaggaaaattaggcagactcctgggaatatttgagaatcaagaccggaagcacagaacatatgtttatgttctgacagtcactgaaatattggaagactgggaagactctgttaacatagggaggaagagagagtggttcaaagtggaagacgcaatcaaggttcttcagtgtcacaagcctgtccacgcagagtacctggaaaggctgaagctggggtgttctccgaccaacgggaattccacggtcccttccctcccagataacaatgccttgtttgtgactgccgcaccaccctctggggtgccatccagtataagatagagagctggggcccctcccccaccgtgtcatggcacatgtcagagggagagaggcttttttacttctaacacatctgactgctgctggcagactctagatttgccatgcaggggtttcaaataatttgcatgtatttcagatgctttcaactcttttaaaataaataacaaaatagcaaaaatactttaagatgccaaagccatgtggattttttttaaagccttaattgtaagtctaccccattactttggttctcatttttataacctttcttaaaatttcacttttgaacactcaacattctatggtttattttgacagatcaactggttgttgggtatgtttttgccctaatatttttttcttacagtatcaaatagtcaccaaatctttgggaattaatgaaaaatattcttggtatttaaaaaaatatatatgtgccccaaaacttgtttaagagagtcgcattctctccacttgtaagctgcaggtttgcgctgatttgtatggacgggccagttgcctttgaccaagcactcaagtttactctatccgagaatttgtacacagcaagaattatgtcagtgcacaggcactgcctcccctctgacacggtgattccatatgaatcactccatttttagggaaaactgtcaggaacttgcctgtggcaccgcacagctcattctgaaactgttcagagaagtctgttcagcacgctctgcggctgcttcctccctgctccttccgacacagactgtgttaggggccacattgctcccatggccagtgctcttcatattttcagtctagcaacaacactgttgaccagtggattagactctaaccttagatacagcagtcactgactggcatccaaggttagatcacactaaacaaaatggctgtcctaggcctaagacactgtcttgagtgtcagtaagcagcttgtcagatgagtgtgagggaactagaactatgttaccttgtctttgtgctctctcaaaacgggccaccccatggaaaagtacctacatttccttcaggaagtcatggaggaagtgaagggggaggggggagaggaaaagatctttcgcagtacagttaggacatctgtaacctgttaacctttaaattgtaatgttgctgctgccaatgtccttacctgtaaggaaggaaaatgagatctcgtgcccaaatgcagcagaactaaaaacattaggttacaaagcagaaaaacccaaccagtagtgttcattctaaattatgtagcgtaaacaaatgtgatcgtccagaccttttctatttttctttgtacagaggtcatgtactctgggtgtgttttaaaagaaacattttaaagaaacccacagattgacattttctatcttttcgttttctcacgattctctgcaatttttccatgtactaaacacaacagaaactggactgttttgtatagaatactgcttccagctaaaaccatcacaagtgcccgggagtttatgtggacatgatagatactctgggcggtaataatgcatgaacctgttttataaactcttgaactgtgtattgccatgtaaaatatgtacagtattaatgtcaaccatctctttaaaagttagcctatgaacattgttgtataatttctttggtcagaaacatttggactaagtcatgtcacctgggtcaggattttcttcaatgccgtgtagcaaaactgtctttagtctatgcaaatagcgagtcactgagtgtgacagaatgcaacttcactgttaaacttcacctgagggttcctcattctggaatccagactgcaagattataaaggaaaagacctaaggcaattcagttctttttgcaaatcaattgaatccacgagagatgtctaccagcgagatgtttaccagcccagccgcctgcagcctgcagcctgctgtgtgtgcttatttgtgcgctgaataaaatggggcagctaaattctccagttccatatgcctccgaagttcaaagaaaagaaagcaaagtaacatgttagacttgacttgtgtggcggggtaaagaaatggcatcttcccactaagaacgaaccatccagttcttttgtcagtcacactatgaaacagggaaggtgaagggaagaaatggttatgtgtgcacgaatcgctttgcatggtctcatgagatggctgcattcgaactgttttaagaattgtaaggatcttgacttttttacatttggaaacatcaaataaaaacaaacataatctgtgaaaaaaaaaaaaaaaaaaaaaaaaa

SEQ ID NO: 67 - protein, 179 aa, Rattus norvegicus
MKFKPNQTRTYDREGFKKRAACLCFRSEQEDEVLLVSSSRYPDQWIVPGGGVEPEEEPGGAAAREVYEEAGVKGKLGRLLGIFENQDRKHRTYVYVLTVTEILEDWEDSVNIGRKREWFKVEDAIKVLQCHKPVHAEYLERLKLGCSPTNGNSTVPSLPDNNALFVTAAPPSGVPSSIR

SEQ ID NO: 68 - DNA, 2050 bp, Rattus norvegicus
  CDS (26)..(1945)
gaggggtggcggacgagctgcagagatgtctggccagagcctgacggaccgaatcaccgcggcccagcacagtgtcactggctccgcggtatctaagacagtatgcaaggccacgacccacgagatcatgggccccaagaaaaagcacctggactacttaattcagtgtacaaatgagatgaatgtgaatatcccacagttggcagacagtttgtttgaaagaactactaatagtagttgggtggtggtcttcaaatcactcattacaactcatcatttgatggtgtatggaaacgagcgtttcattcagtatttggcttcaagaaacacattgtttaacttaagcaactttttggataaaagtggattgcaaggatatgatatgtctacatttattagacgatatagtaggtacctaaatgaaaaggcagtttcatacagacaagttgcattcgatttcacaaaagtgaagagaggagctgatggagttatgagaacaatgaacacagaaaaactgttaaaaactgtaccaattatccaaaatcaaatggatgcacttcttgattttaatgttaatagtaatgaacttacaaatggggtaataaatgctgccttcatgctcctgttcaaagatgccattagactatttgcagcatacaatgaaggaattattaatttattggaaaaatattttgatatgaaaaagaaccagtgcaaagaaggtcttgacatctataagaagtttttgactaggatgacaagaatctcagagtttctgaaagttgcagagcaagttggaattgacagaggagatattccagatctttcacaggcccccagcagtcttcttgatgctttagaacaacatttagcttccttggaagggaagaaaatcaaagattccacagctgcaagcagggctacaacactttccaatgcagtctcttctttggcaagcactggcctatctctgaccaaagtggatgaaagggaaaagcaggcagcattagaggaagaacaggctcgattaaaagcactaaaggaacagcgtctaaaagaacttgcaaagaaaccgcatacctctttaacaactgcagcctctcctgtgtccacctcagcagggggaataatgactgcaccagccatcgacatattttctacccctagttcttctaacagcacatccaagctgccaaatgacctgcttgatttgcagcagccaacctttcatccatctgtccatgctatgtcagctgctcctcaggtagcaagtacatggggagatgctgttgatgatgccattccaagcttaaatcctttcctcacaaaaagtagtggtgatgttcaccttcctatttcttcagatgtatccacttttactactaggacacctactcatgaaatgtttgttggattcagtccttctccggttacacagccacatccttcagctggccttaatgttgactttgaatctgtgtttggaaataagtctacgaatgttgctgtagattctggtggtggacttctcaaaccaacagtggcctctcagaaccagagtcttcctgttgccaaacttccgcctaacaaattagtgtctgatgacttggattcatctttagccaaccttgtgggcaatcttggcattggaaatggaaccactaagaatgatgtaagttgcagtcaaccaggtgaaaagaagttaactggaggatctaactggcaaccaaaggtcgcaccaacaactgcctggagtgctgcaacaatggcaccccctgtaatggcctatcctgctactacaccaacgggcatgataggatatggaattcctcctcagatgggaagtgtacctgtaatgacacagccaaccttaatatacagccagcctgtcatgagaccgccaaacccctttggccctgtaccaggagcacagatacagtttatgtaactagatggaagagaatggaattactccaagaatagaagtgcacaggtggcgactccttacttccagcaaaatccaaactgctgtctctaagactcttcctcc

SEQ ID NO: 69 - protein, 640 aa, Rattus norvegicus
MSGQSLTDRITAAQHSVTGSAVSKTVCKATTHEIMGPKKKHLDYLIQCTNEMNVNIPQLADSLFERTTNSSWVVVFKSLITTHHLMVYGNERFIQYLASRNTLFNLSNFLDKSGLQGYDMSTFIRRYSRYLNEKAVSYRQVAFDFTKVKRGADGVMRTMNTEKLLKTVPIIQNQMDALLDFNVNSNELTNGVINAAFMLLFKDAIRLFAAYNEGIINLLEKYFDMKKNQCKEGLDIYKKFLTRMTRISEFLKVAEQVGIDRGDIPDLSQAPSSLLDALEQHLASLEGKKIKDSTAASRATTLSNAVSSLASTGLSLTKVDEREKQAALEEEQARLKALKEQRLKELAKKPHTSLTTAASPVSTSAGGIMTAPAIDIFSTPSSSNSTSKLPNDLLDLQQPTFHPSVHAMSAAPQVASTWGDAVDDAIPSLNPFLTKSSGDVHLPISSDVSTFTTRTPTHEMFVGFSPSPVTQPHPSAGLNVDFESVFGNKSTNVAVDSGGGLLKPTVASQNQSLPVAKLPPNKLVSDDLDSSLANLVGNLGIGNGTTKNDVSCSQPGEKKLTGGSNWQPKVAPTTAWSAATMAPPVMAYPATTPTGMIGYGIPPQMGSVPVMTQPTLIYSQPVMRPPNPFGPVPGAQIQFM

SEQ ID NO: 70 - DNA, 361 bp, Rattus norvegicus
  CDS (30)..(296)
gcacgaggtgaaaggaaatttttatcgacatggcaactgaactggagaaggccttgagcaacgtcattgaagtctaccacaattattctggtataaaagggaatcaccatgccctctacagggatgacttcaggaaaatggtcactactgagtgccctcagtttgtgcagaataaaaataccgaaagcttgttcaaagaattggacgtcaatagtgacaacgcaattaacttcgaagagttccttgcgttggtgataagggtgggcgtggcagctcataaagacagccacaaggagtaacagagcttctggcctgggctgggtcccttggatatgtctacagaataaagtcgtcatatctt

SEQ ID NO: 71 - protein, 89 aa, Rattus norvegicus
MATELEKALSNVIEVYHNYSGIKGNHHALYRDDFRKMVTTECPQFVQNKNTESLFKELDVNSDNAINFEEFLALVIRVGVAAHKDSHKE

SEQ ID NO: 72 - DNA, 494 bp, Rattus norvegicus
  CDS (32)..(370)
cggcacgagctccttagctttgagcaagaagatggctgccaaaacaggatctcagctggagcgcagcataagcaccatcatcaatgttttccatcagtactctaggaagtatggacatcctgacaccctgaacaaggcggaattcaaagaaatggtgaataaggacttgccaaattttctgaagagggagaaaagaaatgaaaatctcctaagagacatcatggaggacctggacacaaaccaggacaatcaactgtcctttgaggagtgtatgatgctgatgggaaagttgatctttgcctgtcatgagaagctgcatgagaacaacccacgtgggcatgaccacaggcacggcaaaggctgtgggaagtaattaagaggtcgccatgtaacatctgcccaaccaagtctaaagggaatagcttactaaatgaccttggttctggggctgggaaataatttaaaaatgaataaataaagtctttatccattcc

SEQ ID NO: 73 - protein, 113 aa, Rattus norvegicus
MAAKTGSQLERSISTIINVFHQYSRKYGHPDTLNKAEFKEMVNKDLPNFLKREKRNENLLRDIMEDLDTNQDNQLSFEECMMLMGKLIFACHEKLHENNPRGHDHRHGKGCGK

SEQ ID NO: 74 - DNA, 2320 bp, Rattus norvegicus
  CDS (178)..(1341)
gcctagggtggcagcggctacctacctcggatctcgacctgctgccaccacggcctgagcgctgtccctcggctcccggagctcagcgcgaagccctggccccggcggctggggcatggatcaggggcgctgcgtgaggcggcttcctgcacggccgtgacgtgcaccggcttcagcatgaagaccctcatcgctgcctactccggggtcctgcggggtgagcgtcgggccgaagctgcccgcagcgagaacaagaataaaggatctgccctgtcacgcgaggggtctgggcgatggggcactggctccagcatcctctcggccctccaagacatcttctctgtcacctggctcaacagatccaaggtggaaaaacacctacaggtcatctcagtcctacagtgggtcctatccttcctggtgctaggagtggcctgcagtgtcatcctcatgtacaccttctgcactgactgctggctgatagctgctctctacttcacctggctggcatttgactggaacacgcccaagaaaggtggcaggagatcacagtgggtgcgaaactgggccgtgtggcgctattttcgagactactttcccatccagctggtgaagacacacaacctgctgaccaccaggaactatatctttggataccatccccatggcatcatgggcctgggtgccttctgtaacttcagcacggaggccaccgaagttagcaagaagttccctggcataaggccttatttggccacattggctggcaacttccggatgcctgtgcttcgggagtacctgatgtctggaggcatctgccctgtcaacagagacaccatagactacttgctttccaagaatgggagtggtaatgccattgtcatcgtggtgggaggtgcagctgaatccctgagctccatgcctggcaagaacgcagtcaccctgcggaaccgcaaaggctttgtaaagctggccctgcgccatggagctgatctggttcccacctattcctttggagagaatgaggtatacaagcaggtgatctttgaggagggctcctggggccgatgggtccagaagaagttccagaagtatattggtttcgccccctgcatcttccatggccgaggtctcttctcctctgacacctgggggctggtgccctactccaagcccatcaccaccgttgtgggggagcccatcaccgtccctaagctggagcacccgacccagaaagacatcgacctgtaccacaccatgtacatggaggccctggtgaagctctttgacaatcacaagaccaaattcggccttccagagactgaggtgctggaggtgaactgacccagcccatgggtgccagctcctggaaggaatgactgcaaatccttttctaccaagttctcaagtgcattttgttctgtaaatttggaagcgtcatgggtgtctgtgggttatttaaaagaaattataatttgttaaaccattgcaatgttagatcttttttaagaagggaagagtcagtattttaagctcacttctagtgtgtcctgcctaaggtggtggctgacatttataggccttgatggtttctatccaccccttctagtgttccccaaacaacagacacttggccctagttaattggggaagggcagcccttagtgactcagccatttaatcctcttcgctctagggattctcaagagacggaggccacgtttaaaaacacctccattcccacccacaacacgtgactgctggtcaggtttttcttacttaggggaggatgagggggcatagctggttccgcttggggagagtggtagataacatctggaatgcccggctcgagtgtcctctgctccccacctactcctcttctccaatctgagcctaccctggcctcctgtacactgtgctagggacagggctgtcccacaggtgccatgttgggttatctcgctgctgttggctggttttactctggagattggcatcgtgaacacagctcagcgtcattctggagatgttctcccagccacctgagctctcctgagccacaccccaagtctggtgtgaggagaggcctctgttcttcacagaggtgcctggcttcctgtgcagcacactgggtccaggacaggaggccacccccaaccaagcctcacctgtgtgcctttatgaggcgtcgggagaaagccaccctcctgtgtattctgctttctccatgagattttgccatgtcacacttttgtatattcctagactaataaatggaaacaagaacagccaaaaaaaaaaaaaaaaaaaaaaaaaa

SEQ ID NO: 75 - protein, 388 aa, Rattus norvegicus
MKTLIAAYSGVLRGERRAEAARSENKNKGSALSREGSGRWGTGSSILSALQDIFSVTWLNRSKVEKHLQVISVLQWVLSFLVLGVACSVILMYTFCTDCWLIAALYFTWLAFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVNRDTIDYLLSKNGSGNAIVIVVGGAAESLSSMPGKNAVTLRNRKGFVKLALRHGADLVPTYSFGENEVYKQVIFEEGSWGRWVQKKFQKYIGFAPCIFHGRGLFSSDTWGLVPYSKPITTVVGEPITVPKLEHPTQKDIDLYHTMYMEALVKLFDNHKTKFGLPETEVLEVN

SEQ ID NO: 76 - DNA, 1714 bp, Rattus norvegicus
  CDS (56)..(1102)
cctctgaggcgtttggtgctccggtagctaccagggctggagagcgagcattgccatggaaatatacacttcggataactactccgaagaagtagggtctggagactatgactccaacaaggaaccctgcttccgggatgaaaacgaaaacttcaacaggatcttcctgcccaccatctattttatcatcttcttgactggcatagtgggcaatgggttggtaatcctggtcatgggttaccagaagaagctgaggagcatgacagacaagtaccggctgcacctgtccgtggctgacctcctctttgtcatcacactccccttctgggcagtggacgccatggctgactggtactttgggaaatttttatgtaaggctgtgcatatcatctacaccgtcaacctttacagcagtgttctcatcctggccttcatcagcctggaccgctaccttgccattgtccacgccaccaacagccagaggccgaggaagctgctggctgaaaaggccgtctatgtgggtgtctggatccccgccctcctcctgactatccctgacatcatcttcgccgatgtcagccagggggacggcaggtacatctgtgaccgcctttaccccgacagcctgtggatggtggtgttccagttccagcacatcatggtgggtctcatcctgccgggcatcgtcatcctgtcctgttactgcatcatcatctccaagctgtcacactccaagggccaccagaagcgcaaggccctcaagactacggtcatccttatcctggctttctttgcctgctggctaccgtattacgtggggatcagcatcgattccttcatccttttggaggtcatcaagcaaggatgtgagttcgagagcgtcgtgcacaagtggatctccatcacggaggccctcgccttcttccactgttgcctgaaccccatcctctacgccttcctcggggccaaattcaagagctccgcgcagcatgcactcaattccatgagcagaggctccagcctcaagatcctttccaaagggaaacggggtggacactcttccgtctccacagagtcagaatcctcaagttttcactccagctaacacttacgcaaagacatatataatatatatatatatatatatatatgacaaagaactttttttatgttacacatttcccagatataaaagactgaccaatcttgtacagttttttttattgactgttggagtttatgtttctctagtttttgtgtgtgtgtgtgtgtgtgaggtttgacttaatttatataaatattggtttttttctgttgtttgtctgtttcacgtgaatgagtgtctaggcaggacctgtggccaagttcttagtcgctgtgtgtctgtgtgtaggactgtagagctgtagaggaaggaactgaacgctccagaatgtgtggtaaatcggataaagctagaagtgaccctcagctgtcgctgcataatctcttcgccccgcccaccccgttcctgaattgtttggttgtgctgtgtggtttctctgtagaagatggcacttaaaaccaaagcctgaaatggtatagaaatgctgggttttttctttcagttttcaagggtggattgatttcagtacctacaaatgtacagtcttgtattatgttgttaataaaagtcaatggtaaacttgaaaaaaaaaaaaaaaaaaaaaaaaaa

SEQ ID NO: 77 - protein, 349 aa, Rattus norvegicus
MEIYTSDNYSEEVGSGDYDSNKEPCFRDENENFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVYVGVWIPALLLTIPDIIFADVSQGDGRYICDRLYPDSLWMVVFQFQHIMVGLILPGIVILSCYCIIISKLSHSKGHQKRKALKTTVILILAFFACWLPYYVGISIDSFILLEVIKQGCEFESVVHKWISITEALAFFHCCLNPILYAFLGAKFKSSAQHALNSMSRGSSLKILSKGKRGGHSSVSTESESSSFHSS

SEQ ID NO: 78 - DNA, 1329 bp, Rattus norvegicus
  CDS (77)..(880)
gggcggttcaaggcataacaggctcatctgggatcctctccagtcaggcttccttgtgcaagtgtctgaagcagctatggcaactgtccctgaactcaactgtgaaatagcagctttcgacagtgaggagaatgacctgttctttgaggctgacagaccccaaaagattaaggattgcttccaagcccttgacttgggctgtccagatgagagcatccagcttcaaatctcacagcagcatctcgacaagagcttcaggaaggcagtgtcactcattgtggctgtggagaagctgtggcagctacctatgtcttgcccgtggagcttccaggatgaggacccaagcaccttcttttccttcatctttgaagaagagcccgtcctctgtgactcgtgggatgatgacgacctgctagtgtgtgatgttcccattagacagctgcactgcaggcttcgagatgaacaacaaaaatgcctcgtgctgtctgacccatgtgagctgaaagctctccacctcaatggacagaacataagccaacaagtggtattctccatgagctttgtacaaggagagacaagcaacgacaaaatccctgtggccttgggcctcaaggggttgaatctatacctgtcctgtgtgatgaaagacggcacacccaccctgcagctggagagtgtggatcccaaacaatacccaaagaagaagatggaaaagcggtttgtcttcaacaagatagaagtcaagaccaaagtggagtttgagtctgcacagttccccaactggtacatcagcacctctcaagcagagcacagacctgtcttcctaggaaacagcaatggtcgggacatagttgacttcaccatggaacccgtgtcttcctaaagatggctgcactattcctaatgccttccccaggacatgctagggagcccccttgtcgagaatgggcagtctccaggggaagcctttgtcctctgccaagtcaggtctctcagagccataagaaaaccgtggcacattctggtcaaagaaaacgtgtgtttccctccctgcctctgacaggcaaccacttacctatttatttatgtatttattgattggttgatctatttaagttgattcaggggggtcacgaggcagcattgtcgacagaagaatctagttgtccgtgtgtatgggatgaattgaatttggaccagtgcacagccagcactgagttctttcattgatgctgaaaatgaagagtttcatattgtgtggatgagagtgtttatgaatgaagcacaagcacatcattttgatgagtatgaaataaatgtcactaaaac

SEQ ID NO: 79 - protein, 268 aa, Rattus norvegicus
MATVPELNCEIAAFDSEENDLFFEADRPQKIKDCFQALDLGCPDESIQLQISQQHLDKSFRKAVSLIVAVEKLWQLPMSCPWSFQDEDPSTFFSFIFEEEPVLCDSWDDDDLLVCDVPIRQLHCRLRDEQQKCLVLSDPCELKALHLNGQNISQQVVFSMSFVQGETSNDKIPVALGLKGLNLYLSCVMKDGTPTLQLESVDPKQYPKKKMEKRFVFNKIEVKTKVEFESAQFPNWYISTSQAEHRPVFLGNSNGRDIVDFTMEPVSS

SEQ ID NO: 80 - DNA, 2924 bp, Rattus norvegicus
  CDS (9)..(2174)
gcgggcgcatgcgcagggggtgcgcgccgcctctgccccgcggcgagggtgtctatggagaggcggcggccgcggctgctgaggcggacgctgaggcagtggcgatggcgcccttccccgatgaagtggacgtcttcaccgccccacactggcggatgaagcagctggtggggcgctactgcgacaagctttctaaaaccaacttttccaacaacaatgacttccgtgctcttctccagtctttatatgctactttcaaggagtttaaaatgcatgaacaaattgaaaatgaatacattattggtttacttcagcaacgcagtcagaccatttacaatgtgcattctgacaataagctctccgagatgcttagtctctttgaaaaggggctgaagaatgttaagaatgagtgtgaacagttgaattatgcaaagcagctgaaggagagattggaggccttcacgagagatttccttccccacatgaaagaggaggaggaggtttttcagcccatgttaatggaatattttacctatgaagagcttaaagatatcaaaaagaaagtgatcgcacaacactgctcacagaaggacacagcagaactcctcagagggctcagtctgtggaatcaagctgaagagcgacagaaagttttcaaatattctgtggatgagaagtcagatgcagaagcggaagtgtcagaacactccacaggtataacccatcttcctcctgaggtaatgctgtccattttcagttaccttaatcctcaagaattgtgtcggtgcagtcaagtcagcactaagtggtctcagctggcaaagacaggatctttgtggaaacatctttaccctgttcattgggcaagaggtgactggtatagtggccctgccactgaacttgatactgaacctgatgaggaatgggtgagaagtaggaaagatgaaagtcgtgctttccaggaatgggacgaagatgccgatatagatgaatctgaagagtctgcagaggaatcggttgctatcagcattgcacaaatggaaaaacgtttactccatggcttaattcataatgttctgccatatgttggtacctctgtaaaaactttagtgttagcatacagctctgcagtctccagcaaaatggttaggcagattttagagctttgtcctaatctggaacatctggatcttacccagactgacatttccgattctgcatttgacagttggtcttggcttggctgctgccagagtcttcggcatcttgacttgtctggatgtgaaaaaatcacagacatggctctggagaagatttctagagctcttggagttctgacatctcatcagagtggcgttctgaaaagtgcaggcaagactacttcaactccatggacaaataaagacattaccatgccatccaccacgcagtatgcctgtttgcacaatttaactaacaaaggcgttggtgaagaaatcgacagcgaacacacttggactgaacctgtgtcttctgaaagtttcactcctccctacgtgtggatgctagatgctgaagatttggccgatatcgaagatgctgtagaatggaggcacagaaacgttgaaagtctctgtgtgatggaaacagcctccaactttggttgttcctcctctggttgttacagcaaggacattgtgggattaaggactagtgtctgttggcagcagcattgtgcttctccagcctttgcgtattgtggtcattcattctgttgcacagggacagctctaagaactatgtctgcactcccagcggcttctgcagtgtgtagaaaagcattaaggactacgttgcccagggggaaagacttaatttattttgggagtgaaaagtctgaccaagagactggacgagtacttttgttcctcagtctgtctggatgttaccagatcacagaccatggtctcagggttctgactctgggaggaggactgccttacttggagcacctgaatctctctggctgtctgactgtaactggtgcaggactgcaggatttggtctcagcatgtccttccctaaatgacgaatacttctactactgtgacaacattaacggtcctcatgctgacaccgccagtggatgccagaatttgcagtgtggttttcgagcctgctgccgctctggcgaatgacccttgacttctgacctttgtctacttcatttagctgagcaggcttcctttcatgcactttacttatagcacatttcttgtgttaacccattttgggtgtgtcttgttttggccccatttcttacagtctcagaaatttaatttagcagtgaactgtacaatgttgtttttctttgcaaaatatatttctgtttagaaaagattaggattttttgtagggtgagaccagtcttctggtttcttttaaatgaaatgcttaaaaagaatagcactaataccatgctatttaaaatattttagatttatttacatttagtcaatggggggttattggttctcttatgtgtaaacactgtaccaagttttgcagctcctttcaaatatacatttctgaagatttattttcaaagtctgaacattttagaaatgaatctcttttcccctaatgttttctttatttcaacaattctcaggggccaattcaaacacacttagaagcgagattctttaagattatagaataagttggcctcttggtgccggcccagtgagccagcagaacagcagtctgtttctattgctgtagacacctgcttccacggctgtccctaaactgttgaattcgggaggtatttttaaaaattccacaattgtttgaagaaatgtataaataaaatctactttgagaactttaccaagcaatatcttttgtgttgtgattttattttaaatctcaatctaagttta

SEQ ID NO: 81 - protein, 722 aa, Rattus norvegicus
MRRGCAPPLPRGEGVYGEAAAAAAEADAEAVAMAPFPDEVDVFTAPHWRMKQLVGRYCDKLSKTNFSNNNDFRALLQSLYATFKEFKMHEQIENEYIIGLLQQRSQTIYNVHSDNKLSEMLSLFEKGLKNVKNECEQLNYAKQLKERLEAFTRDFLPHMKEEEEVFQPMLMEYFTYEELKDIKKKVIAQHCSQKDTAELLRGLSLWNQAEERQKVFKYSVDEKSDAEAEVSEHSTGITHLPPEVMLSIFSYLNPQELCRCSQVSTKWSQLAKTGSLWKHLYPVHWARGDWYSGPATELDTEPDEEWVRSRKDESRAFQEWDEDADIDESEESAEESVAISIAQMEKRLLHGLIHNVLPYVGTSVKTLVLAYSSAVSSKMVRQILELCPNLEHLDLTQTDISDSAFDSWSWLGCCQSLRHLDLSGCEKITDMALEKISRALGVLTSHQSGVLKSAGKTTSTPWTNKDITMPSTTQYACLHNLTNKGVGEEIDSEHTWTEPVSSESFTPPYVWMLDAEDLADIEDAVEWRHRNVESLCVMETASNFGCSSSGCYSKDIVGLRTSVCWQQHCASPAFAYCGHSFCCTGTALRTMSALPAASAVCRKALRTTLPRGKDLIYFGSEKSDQETGRVLLFLSLSGCYQITDHGLRVLTLGGGLPYLEHLNLSGCLTVTGAGLQDLVSACPSLNDEYFYYCDNINGPHADTASGCQNLQCGFRACCRSGE

SEQ ID NO: 82 - DNA, 630 bp, Rattus norvegicus
  CDS (20)..(568)
cgtgtgcagcacgtccagcatgttgtttgcctgggctcccttccctgccctcctgggcctggcagactcctgctgtttcgtcgtgccccgcagtgagtggaaggccctgccatccgagtgctccaagggcctgaagaaaccagtccgctacgtggtgatctcacacacagccggcagcttctgcagcagcccagactcctgtgaacagcaggcccgcaatgtgcagctttaccaaatgaaacagctgggctggtgcgatgtagcctacaacttcctcattggagaggatggtcatgtctacgaaggccgaggctggaccatcaagggtgaccacacagggcccatctggaaccccatgtctatcggcatcaccttcatgggtgactactcacaccgggtacctgcaaagcgggctctccgtgctgccctaaatcttctgaaatgtggggtgtctgagggcttcctgagatctaactatgaggtcaaaggacatcgagatgtgcaaagcactctgtctccaggtgaccagctctacgagatcatccaaagctgggaccactaccgagagtgagatcttgagaccttgtggagttcccaccctgaaatccctcctaccacctgctgcttccc

SEQ ID NO: 83 - protein, 183 aa, Rattus norvegicus
MLFAWAPFPALLGLADSCCFVVPRSEWKALPSECSKGLKKPVRYVVISHTAGSFCSSPDSCEQQARNVQLYQMKQLGWCDVAYNFLIGEDGHVYEGRGWTIKGDHTGPIWNPMSIGITFMGDYSHRVPAKRALRAALNLLKCGVSEGFLRSNYEVKGHRDVQSTLSPGDQLYEIIQSWDHYRE

SEQ ID NO: 84 - DNA, 1393 bp, Rattus norvegicus
  CDS (98)..(973)
ggggggcgtgattccgtcctgcgtgtctgttctgcggaacagtaggcagttgttttccgtccggcttctctcacactcaagtgcgcgcctccacctcatggaagactcgatggacatggacatgagccctcttaggcctcagaactaccttttcggttgtgaactaaaggctgacaaagattatcactttaaagtggataatgatgaaaatgagcaccagttatcattaagaacggtcagtttaggagcaggggcaaaagatgagttgcacatcgtagaggcagaagcaatgaactatgaaggcagcccaattaaagtaacactggcaactttgaaaatgtctgtacaaccaacagtttcccttgggggcttcgaaattacaccacctgtggtcttgaggttgaagtgtggttctgggcctgtgcacataagtggacagcacctagtagctgtagaggaagatgcagagtcagaagatgaagatgaggaagatgtaaaactcttaggcatgtctggaaagagatctgctcccggaggtggtaacaaagtcccacagaaaaaagtaaaacttgatgaagatgatgatgaggatgatgaagatgatgaggatgatgaagatgatgatgatgatgattttgatgaagaggaaactgaagaaaaggttccagtgaagaaatctgtacgagataccccagccaaaaatgcacaaaaatcaaaccaaaatgggaaagatttaaaaccatcaacaccaaggtcaaagggtcaagagtccttcaaaaaacaggaaaaaactcccaaaacacccaaaggacctagctctgtagaagacattaaggcaaaaatgcaagcaagtatagaaaaaggtggttctcttcccaaagtggaagccaagttcattaattatgtgaagaattgtttccggatgactgaccaggaggctattcaagatctctggcagtggaggaagtctctttaagaaaatggtttaaacagtttgaaataattctgtcttcatttctgtaatagttgctatctggctgtcctttttataatgcaaagtgagaactttccctactgtgtttgataaatgttgtccaggttcaattgccaagaatgtgttgtctaaaatgcctgtttagttttcaaggatggaactccgccctttacttggttttaagtatgtatggaatgttatgataggacatagtagtagtggtggtcagatgtggaaatggtagggagacaaatatacatgtgaaataaactcagtattttaataaagtaaaaaaaaaaaaaaaaaaaaaaaaaaaaaaaaaaaaaaaaaaaaaaaaaaaaaaaaaaaaaaaaaaaaaaaaaaaaaataaaaaaaaaaaaaaaaaaaaaaaaaaaaaaa

SEQ ID NO: 85 - protein, 292 aa, Rattus norvegicus
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEDEEDVKLLGMSGKRSAPGGGNKVPQKKVKLDEDDDEDDEDDEDDEDDDDDDFDEEETEEKVPVKKSVRDTPAKNAQKSNQNGKDLKPSTPRSKGQESFKKQEKTPKTPKGPSSVEDIKAKMQASIEKGGSLPKVEAKFINYVKNCFRMTDQEAIQDLWQWRKSL

SEQ ID NO: 86 - DNA, 1326 bp, Rattus norvegicus
  CDS (10)..(1005)
ggagcagccatggcctcttggcagcatcaagggagaaccctgttcctcctggccctcttgggcggggtgtcgggcctcagggaatgcttgatactgcagtcggttaagggaagcacggtctcctgccacggtcccactgagttcccgagctccctcccggccgacactgtccatctgtcggtggaattctccagcctgactcagctgcccgccgccgccctgcaagggtgtcccggtctgcaggaactgcacctctccagcaaccgcctgcaggaactgtcccccgggctgctggcgcccgtgccccggctgcgcgtcctggatctgactcgcaatgctctccgcagcctgccccccggtttgttccgcagctcggccgccctgaacaccctggtcctgagggagaaccagctgcaggaggcgagcgcgcgctggctgcagggcctggacgccctgggctacttggacctggcggagaaccggctgcgctcgctgcccgctaggctccttgccaacctaggcgccctgcacaccctcgaccttgggcacaacctgcttgagtcgctgcccgagggattcctgaggggccccaggcggctacagcgcctgcacctggaagggaaccggctgcaaaggctggaggctggcttacttgcgccccagccgttcctgggcgtcctgttcctgaatgacaaccagctaaccgcagtggcggccgactcctttcggggcctaaagcagctggatatgttggatctgtccaataactctctgtccagcactcccccgggtctgtgggcgttcctggggagaccgacccgcgatatgcaggatggtttcgatgtctcccacaacccctgggtctgtgacaaggacctcgtggacctgtgccgctggctggccgccaaccgacataagatgttctcgcagaacgacacactctgtgcggggcccgaggccgtgaggggacagcgactgctggacgtggcagagctggggaccttgtgaggatggcaactggggtgcgagccaagggtaccccgcttgccactgaagcaatttggtcccatgtcagaatgcagattcccagcatctgccattccccattccctcagccaggaatgctattccctgactctccctcaggctcctctccatttgccccaactcttccacctctcactgttcctgtgctggcccccaggctaccatgtgtttatctagctttgcctcatatgtttcagggtcaccaaagcagttaataaaacagctcccggctggctgagccgctcagcgtcaaaaaaaaaaaaaaaaaaaaaaaaaaaa

SEQ ID NO: 87 - protein, 332 aa, Rattus norvegicus
MASWQHQGRTLFLLALLGGVSGLRECLILQSVKGSTVSCHGPTEFPSSLPADTVHLSVEFSSLTQLPAAALQGCPGLQELHLSSNRLQELSPGLLAPVPRLRVLDLTRNALRSLPPGLFRSSAALNTLVLRENQLQEASARWLQGLDALGYLDLAENRLRSLPARLLANLGALHTLDLGHNLLESLPEGFLRGPRRLQRLHLEGNRLQRLEAGLLAPQPFLGVLFLNDNQLTAVAADSFRGLKQLDMLDLSNNSLSSTPPGLWAFLGRPTRDMQDGFDVSHNPWVCDKDLVDLCRWLAANRHKMFSQNDTLCAGPEAVRGQRLLDVAELGTL

SEQ ID NO: 88 - DNA, 2216 bp, Rattus norvegicus
  CDS (7)..(1995)
tgcaagatgggtgtctaccgcatccgcgtctccaccggagactccaagtacgcgggctccaacaacgaggtctacctgtggttggttggacagcatggagaggcatctctcgggaagctgctacgaccctgtcgggactcggaagcagaattcaaagtggatgtgtcaggataccttgggccactgctgttcgtaagagtgcagaaatggcattatctcacggatgacgcctggttctgcaactggatttctgtgaagggccccggagaccaaggatcagagtacatgttcccctgttaccgatgggttcagggcagaagcatcctgagcctccctgagggcactggctgcaccgtggttgaagattctcaaggactgttcaggaaacatagggaagaggagcttgaagagaggaggagtctgtacaggtggggcaactggaaggatggctcaatcctgaatgtggcggcggccagtatatctgacctccctgtagaccaacgatttcgagaggacaaaagaattgaatttgaagcttcacaggttataggggtaatggatactgttgtcaactttcctataaacactgtgacctgctggaaaagcctagatgacttcaactgcgttttcaagagtggccataccaaaatggctgagcgggttcgaaactcctggaaggaagatgcgttctttgggtaccaattcctcaatggtgctaaccccatggtgctgaagcgctctacttgtcttcctgcccgcctggtattccctccaggaatggagaagctacaggcccagctgaacaaggagctccagaaaggcactctgtttgaagcggatttcttccttctggatgggatcaaggccaatgtcatcctttgtagccagcagtacctggctgcccctctcgtcatgctgaagctgatgcctgatggacaactcttgcccatagccatccagcttgaacttcccaaaactgggtctactccaccacctattttcacgccctcggatcccccaatggactggctcctagccaaatgctgggtccggagctccgacttacagctccatgagctgcaggctcatcttctgaggggacacttgatggctgaggtctttgctgtggccaccatgaggtgcctgccttccgtgcaccctgtttttaagcttctagttcctcatctgctttacaccatggaaattaatgtccgggccaggagtgacctgatctcagagagaggcttttttgacaaggcaatgagcacaggtgggggaggccacctggatcttctcaagcaagctggagcctttctgacctattgctcattgtgtccccccgatgacttggctgagcgaggactcttggatatcgagacttgcttctatgctaaagacgccctgcgactctggcagatcatgaatcggtacgtggtgggaatgttcaatctccactacaagaccgacaaagctgtgcaagacgactatgaactgcagagctggtgtcgagagatcactgacattggtcttcaaggggcccaggacagaggcttccctacctctcttcagtcccgggctcaggcttgctacttcatcaccatgtgcatcttcacgtgcaccgcacagcactcttccgtccatcttggccagctggattggttctactgggttcctaatgcaccctgcaccatgcggctgccaccacccaccaccaaggaagcaacaatggagaagctgatggctacactgcccaaccctaatcagtctactctccagataaatgtcgtttggctcctgggcagacgccaggctgttatggtgcccctgggccagcattcagaggaacactttccaaaccctgaggccaaggctgtgctgaagaagttcagagaggaactggctgccttggataaggaaattgagattcgtaataagagcttggacataccttatgagtacctgcggcccagcatggtggaaaacagcgtggccatatgagcatccccagactcctgctttgttgtttagttaagaccacccaagtacatccctgtgcttgagtggtggcagtcctgccctcccaagccccgccctctgaccctcaaagccccaccccctgcccatgtgggaccctcctccaagctgtccattgtctgctgcaatgaatacattttgctttggatgcattaaaaaaaaaaaaaaaaaaaaaaaaaaaa

SEQ ID NO: 89 - protein, 663 aa, Rattus norvegicus
MGVYRIRVSTGDSKYAGSNNEVYLWLVGQHGEASLGKLLRPCRDSEAEFKVDVSGYLGPLLFVRVQKWHYLTDDAWFCNWISVKGPGDQGSEYMFPCYRWVQGRSILSLPEGTGCTVVEDSQGLFRKHREEELEERRSLYRWGNWKDGSILNVAAASISDLPVDQRFREDKRIEFEASQVIGVMDTVVNFPINTVTCWKSLDDFNCVFKSGHTKMAERVRNSWKEDAFFGYQFLNGANPMVLKRSTCLPARLVFPPGMEKLQAQLNKELQKGTLFEADFFLLDGIKANVILCSQQYLAAPLVMLKLMPDGQLLPIAIQLELPKTGSTPPPIFTPSDPPMDWLLAKCWVRSSDLQLHELQAHLLRGHLMAEVFAVATMRCLPSVHPVFKLLVPHLLYTMEINVRARSDLISERGFFDKAMSTGGGGHLDLLKQAGAFLTYCSLCPPDDLAERGLLDIETCFYAKDALRLWQIMNRYVVGMFNLHYKTDKAVQDDYELQSWCREITDIGLQGAQDRGFPTSLQSRAQACYFITMCIFTCTAQHSSVHLGQLDWFYWVPNAPCTMRLPPPTTKEATMEKLMATLPNPNQSTLQINVVWLLGRRQAVMVPLGQHSEEHFPNPEAKAVLKKFREELAALDKEIEIRNKSLDIPYEYLRPSMVENSVAI

SEQ ID NO: 90 - DNA, 5487 bp, Rattus norvegicus
  CDS (1)..(4794)
atgattccctcatacccagttgtcctccatggacatgtggccactctgtgccagcagtttttaaaggagctgaacactcacattagagccaccaaggggtgctttctggatggccttgaagccaccacgcggtatgagtccacggccaagtccaagacttcagatggacatactcaatctgagaacagcccctttgaccccttctacacagaagtctgtcggtctgctgctctgcagatggtactctcttgtccaccgctggtcctgggttacactgacaaggttcccagtgtctctgaagctgttcatgttctgtcacatagaacacagttcagggccagccatgtacagttccactgggggaaacaccgtgctaggcacgtggaccacaactcaggcccaatacacacgtcatctctcctgatggaaactgcgcattgtaatcttgcttttcctttctggatctccccacagagagaacttgatgccaccgcaacagtattggcaaacaggcaagatgagagcgaacagtccagaaagcggctcattgagcagagccgagaattcaagaagaacactccagaggatttacgcaagcaggtagcaccactgctcaagagcttccaaggggagattgatgcactgagtaaaaggagcaaagaagcagaagcagccttcttgactgtgtacaagagactaattgatgttccagatcccgtaccagccctggacctcgggcaacagctggaaataaaagtgcagcgtctacacgacattgaaacagagaaccagaaacttagggaaacactagaagagtacaacaaggagtttgctgaagtgaaaaaccaagaggttacgataaaagcacttaaggagaaaatccgagaatacgagcagaccctgaagagtcaggccgagaccatcgctctggagaaggagcagaagctacaaaatgattttgcagagaaggagagaaagctgcaagagacacagatgtccaccacctcaaagctggaggaagctgagcacaaactccagactctgcaaacagccctggaaaaaactcgaacagaattatttgacctgaaaaccaaatacgatgaagaaaccactgcaaatgggcctggggtggagaaggagggtcgtggaacaccttctagaccaccagggcagagttcagtgaagacaagtttaaattgcgatgaaaagctagatcccttctacctaagggccgacgagatcgagatgattatgactgaccttgaacgagccaaccagagggcagaggtggcacagagagaggcagagactttaagggaacagctttcatcggccaaccactctctccaactggcctcgcagatccagaaggctccagatgtggagcaggccatagaggtgctgacccgatccagcctggaagtagagttggctgccaaagagcgggagattgcccagctggtggaagatgtgcagcgactccaggccagcctcaccaagctgcgtgagaattccgccagccagatctcacagctggagcagcagctgaacgccaagaatagcacactcaaagactcaaccaagcccctggaggttttactcctggagaagaaccgctcgctgcagtccgagaatgccacgctgcgcatctccaacagtgacctgagcggaaagtttgcactaaactctcttctccagcgacagctaatgcagtccttctactccaaggccatgcaggaagccggaagcacaagcaccattttttcaacaggtccttacagcacaaactccatatcttccccaagtccattacaacaaagcccagatgtaaatggcatggccccatctcccagccaatcagaaagtgctgggagcatctcagagggtgaggaaattgatacagcagaaatcgcccggcaggtcaaagagcaactgatcaagcacaacattggacagcgcatttttggacattacgtcttgggactatctcaagggtctgtgagtgagattctggcccggccgaagccctggaataagctgactgtccgtggcaaggagccattccacaaaatgaagcagttcttgtctgatgagcagaacatcctggcacttcgaagcatccaaggcagacagagagagaatccaggccagagcctgaacagactctttcaggaagtaccgaaacgaagaaatgggtctgaaggtaacatcacgacccggatccgagcatctgagactggttctgatgaagccatcaagtccatcctagaacaagccaagagggaactccaagtgcagaaaaccgctgagccagtccaggcgtcttccacagccagcagtgggaactccgacgatgccatccgctccatcctacagcaggcccgccgggaaatggaggcccagcaggctgcccttgaccccgccttaaagccagcaccactgtcccagcccgacctcaccatcctcaaccccaagctcctgtctgcctcacccatgtctactgtgtccacttacccccctcttgccatctccctgaagaaaacccctgcagcacctgaggccagtacctcagccttgcccagtgccccagccctcaaaaaagaggctcaggatgcacccacactggacccaccaggatcagctgatgctaccccgggggtactgaggccggtgaagaatgagctagtccgtggcagcacctggaaggatccttggtggaaccctgtacagcctgaaagaagaaatcttaccacttccgaagagaccaaggccgatgaaacaaatgcaagtgggaaagaaaagacaggcagcagccaacctcgggctgagcgcagccagcttcagggaccctcggcgacagcagagtactggaaagagtggcccaacgctgagtccccatactcccagagctcagagctgagcctcactggagccagccgcagtgagacaccccagaatagcccactgccctcttccccaattgtgcccatggcaaagccggccaagccttccgtgcccccgctgactcctgagcagtatgaggtctacatgtatcaggaagtggacaccatcgagctcactcggcaggtcaaagagaagttggccaaaaatggcatttgccagaggatctttggggagaaggtactgggcctgtcccagggcagcgtcagcgacatgctctcaaggccaaaaccatggagcaaactgacccagaaaggccgagaacccttcatccggatgcagctctggctgaatggagagctgggccaaggtgttctgcctgtgcaagggcaacaacaaggaccagtccttcactcagtgacgtctctgcaggaccccctccagcagggctgtgtgagttcagaaagcactccaaagacctctgccagctgcagccctgcccctgagtccccaatgagttccagcgaatctgtgaagagtctcactgagctggtccaacagccctgtcccaccattgagaccagtaaagaaggcaaaccaccagaacccagtgacccacccacctcagactcccaacccacaacaccgctgcctctctctggacactcagccctcagcatccaagaattagtagccatgtctcctgagctggacacctatggcataaccaagagggtcaaagaggtgctgacggacaacaacctcggtcagcgcttatttggagagaccattctggggctcacccagggttctgtctccgacctccttgcccgcccaaagccctggcataagcttagtctgaaaggacgggagccctttgtccgcatgcagctatggctcaacgaccccaacaatgtggagaagctgatggatatgaagcggatggagaagaaagcctacatgaagaggcgacacagctctgtcagtgacagtcagccttgtgaacccccctctgttggtatcgactatagccagggtgccagcccccagccacaacaccagctgaagaaacctcgagtggtcctggctccagaggagaaggaagcactgaagcgagcatatcagcagaagccatacccatcaccaaaaaccatcgaggagcttgccacacaactcaacctgaagactagcaccgtcatcaactggttccataattacaggtctcggatccgcagagaactgttcattgaggagattcaagccggaagccagggccaggccggtgccagcgactcaccttcagctcgaagtagccgggctgcacccagctctgaaggcgacagttgtgacggcgtggaagccgctgacaccgaggagccaggtggcaacatagtggccaccaagtctcagggagggccggcagaggtgaccgcagccccggcagaccgggaagaagcgacgcagccggccgagaaggcgaaggcacagccactatcctcggggaccccaggacaagacgacggtgaagacgcaggccggtctaggccacctcctgagggtcttgctgacgccccagcgcccgtgccaaacctcgccgcacccgcggccggagaggacgccgctacctcagccactgccccagccatggccactgaggcccctggggcggccagggccggccctgccgagaggagttctgcgttgccaagcaccagcgcgcctgccaacgcgcctgcgcgaaggcctagctctctgcagagcctctttggtctgcctgaggcagcgggcgcccgggacaacccggtgcggaagaagaaggccgcgaacttgaacagcatcatccaccgcctggagaaggctgccagccgggaggagcccattgaatgggagttctgagggcgccgggcctctgggcctgaccagcagggcctcgccgggctagccaggcctacttgcgcctagcaggtcccccaggcctagtcagcactggctagtggtgggctggggcaggcgggtgcgtggagggctgtgctgacccgaggcctggacgtgttgcgcacttaccgccctgcgggccacagggcaaaatcgccataggccaaggtgcatatagaaaacaaaggagcattaagcccaatctatgttgtgttttcaaggaagaaaacggaaatgtgtagtcaagcttttttgtaccctgaagtgttttttttttttaattgccctaagtgatttccacaggttctggaataactctttacagcttttgccttgcgccctcctctttcgtgtgggctttaaaaaaaaaaaaaatcaaacccacatattaaaagggggctttttatctgccatctaatggcttcagagcgataatacactattatcttcttaaaccaggaagggggggattttttcagaaaaattaaaaaagaaagtttttgtagctgttcagttgccactaagagattgcacagtcaaaccgactctaaacacactagtttggattcctaaatattttcaagaaaagaatcttctcgtttgaaactttgaattaaaataaaacacatttactccacatattttttaaca

SEQ ID NO: 91 - protein, 1598 aa, Rattus norvegicus
MIPSYPVVLHGHVATLCQQFLKELNTHIRATKGCFLDGLEATTRYESTAKSKTSDGHTQSENSPFDPFYTEVCRSAALQMVLSCPPLVLGYTDKVPSVSEAVHVLSHRTQFRASHVQFHWGKHRARHVDHNSGPIHTSSLLMETAHCNLAFPFWISPQRELDATATVLANRQDESEQSRKRLIEQSREFKKNTPEDLRKQVAPLLKSFQGEIDALSKRSKEAEAAFLTVYKRLIDVPDPVPALDLGQQLEIKVQRLHDIETENQKLRETLEEYNKEFAEVKNQEVTIKALKEKIREYEQTLKSQAETIALEKEQKLQNDFAEKERKLQETQMSTTSKLEEAEHKLQTLQTALEKTRTELFDLKTKYDEETTANGPGVEKEGRGTPSRPPGQSSVKTSLNCDEKLDPFYLRADEIEMIMTDLERANQRAEVAQREAETLREQLSSANHSLQLASQIQKAPDVEQAIEVLTRSSLEVELAAKEREIAQLVEDVQRLQASLTKLRENSASQISQLEQQLNAKNSTLKDSTKPLEVLLLEKNRSLQSENATLRISNSDLSGKFALNSLLQRQLMQSFYSKAMQEAGSTSTIFSTGPYSTNSISSPSPLQQSPDVNGMAPSPSQSESAGSISEGEEIDTAEIARQVKEQLIKHNIGQRIFGHYVLGLSQGSVSEILARPKPWNKLTVRGKEPFHKMKQFLSDEQNILALRSIQGRQRENPGQSLNRLFQEVPKRRNGSEGNITTRIRASETGSDEAIKSILEQAKRELQVQKTAEPVQASSTASSGNSDDAIRSILQQARREMEAQQAALDPALKPAPLSQPDLTILNPKLLSASPMSTVSTYPPLAISLKKTPAAPEASTSALPSAPALKKEAQDAPTLDPPGSADATPGVLRPVKNELVRGSTWKDPWWNPVQPERRNLTTSEETKADETNASGKEKTGSSQPRAERSQLQGPSATAEYWKEWPNAESPYSQSSELSLTGASRSETPQNSPLPSSPIVPMAKPAKPSVPPLTPEQYEVYMYQEVDTIELTRQVKEKLAKNGICQRIFGEKVLGLSQGSVSDMLSRPKPWSKLTQKGREPFIRMQLWLNGELGQGVLPVQGQQQGPVLHSVTSLQDPLQQGCVSSESTPKTSASCSPAPESPMSSSESVKSLTELVQQPCPTIETSKEGKPPEPSDPPTSDSQPTTPLPLSGHSALSIQELVAMSPELDTYGITKRVKEVLTDNNLGQRLFGETILGLTQGSVSDLLARPKPWHKLSLKGREPFVRMQLWLNDPNNVEKLMDMKRMEKKAYMKRRHSSVSDSQPCEPPSVGIDYSQGASPQPQHQLKKPRVVLAPEEKEALKRAYQQKPYPSPKTIEELATQLNLKTSTVINWFHNYRSRIRRELFIEEIQAGSQGQAGASDSPSARSSRAAPSSEGDSCDGVEAADTEEPGGNIVATKSQGGPAEVTAAPADREEATQPAEKAKAQPLSSGTPGQDDGEDAGRSRPPPEGLADAPAPVPNLAAPAAGEDAATSATAPAMATEAPGAARAGPAERSSALPSTSAPANAPARRPSSLQSLFGLPEAAGARDNPVRKKKAANLNSIIHRLEKAASREEPIEWEF

SEQ ID NO: 92 - DNA, 794 bp, Rattus norvegicus
  CDS (50)..(682)
tggatgctgtgagggcggcggcactggttactgcgtgtggcggcccgcgatgaagccgcccatttcaatacaagcaagcgagtttgattcttcagacgaagagcctgctgacgacgagcagactccaattcaaatttcatggctacctctgtcacgagtgaattgttctcagtttctcggtttatgtgctcttccaggttgtaaatttaaagatgttagaagaaacattcaaaaagatacagaggaactaaagagctctgggatccaagacgtatttgttttctgcaccagaggggaactgtcaaaatatagagtcccaaaccttctggacctctaccaacaatatggaattgtcacccatcatcatccgatcccagatggagggactcccgacattggcagctgctgggaaatcatggaggagctggccacctgccttaaaaacaaccgaaaaaccctgatacactgttatggaggacttggaagatcttgtcttgctgcttgtctcctcctatacttgtctgactcaatttcaccacagcaagccatagacagccttcgagatgtcaggggatctggggcgatacagaccatcaagcaatataactatcttcatgaattccgggacaagttagctgcctatctgtcatcaagagattcactgtcaagatccgtgtccagataaggaagagcccagacacgcagccgcagctgaagttccaagttctctagcgtaatctgtattggagtgctactgccatcctgaacataaatgttatgtgcagacttctaaa

SEQ ID NO: 93 - protein, 211 aa, Rattus norvegicus
MKPPISIQASEFDSSDEEPADDEQTPIQISWLPLSRVNCSQFLGLCALPGCKFKDVRRNIQKDTEELKSSGIQDVFVFCTRGELSKYRVPNLLDLYQQYGIVTHHHPIPDGGTPDIGSCWEIMEELATCLKNNRKTLIHCYGGLGRSCLAACLLLYLSDSISPQQAIDSLRDVRGSGAIQTIKQYNYLHEFRDKLAAYLSSRDSLSRSVSR

SEQ ID NO: 94 - DNA, 426 bp, Rattus norvegicus
  CDS (1)..(423)
atgtcggaggtgcggctgccaccgctgcgcgccctagacgactttgtcctggggtctgcacgtttggcagctcctgatctaggcgatccacaacgatggtgtcaccgcgtcatcaacaacctcctctactaccaaaccaattaccttctctgctttggcatcagtctcgctctggccggtcaccctgctgcctgcctggctgcagtacttgccgttagcctcttcattctctgggccgtgggcggcgctttcaccttcctgctctgcatcacagcgcctgtgcttttgatcctgctgcatgcttcactgcgcctgagaaaccttaaaaacaagattgagaacaagatcgagagcatcggtctaaagcggacaccaatgggactgcttctagaggcactgggacaggagcaggaagctggatcctag

SEQ ID NO: 95 - protein, 141 aa, Rattus norvegicus
MSEVRLPPLRALDDFVLGSARLAAPDLGDPQRWCHRVINNLLYYQTNYLLCFGISLALAGHPAACLAAVLAVSLFILWAVGGAFTFLLCITAPVLLILLHASLRLRNLKNKIENKIESIGLKRTPMGLLLEALGQEQEAGS

SEQ ID NO: 96 - DNA, 2210 bp, Rattus norvegicus
  CDS (213)..(497)
ggaaacctcatggccaagttgtagaaaaaatagttgaagtgtgcctatcttggagcggagcccctcccttctccagccgcgcggagtgggagccctaggtgcaaccaagtagccgggttcacggtccagaagctggtgaccctttcctacccaccccaccccacccccgcctcagtgcgccccaccgcgtttccccctgccgccgcgtggccatgagcagccccgtcagaacgccacctcctgagcgcctggagcccaacccagggatgtcctatgccgttatggagattgccatcattgcagctgtgatcactgcggtggccttggtcctggtctgcctcctctttctcatgcttcgctacctgtacaggcacaagggcacttactacaccaatgaggccaaaggcacggagtttgcagaaagtgcggatgcagctctgcaaagtgaccctgccctccaggatgccggggacaccagcaagaaggaatactttatctgaggggtagcagacttgacatccctgcaagacgcttctggctgcaggagagaaagtacccccgctgtgtgccaccattatcagcaccaggaagccctcaagggcaccccaagactcttactgcagaacactgggtgcctgccaggaaatcgatacagatgggaggaatcaaggtgcagcgtacctctcccagggactcattggccacccaataccattggctccgcaaggaagggagaacatctggatgcacaggtctccctgggcaatgcaggtgtcgtctcactagcatgaaaatcctcctcagtgtgtgtgaggtggtttggacctgggagtaagttgtatggaagtgggacatctggaagctgcctgaagataaaggttccaagtgtcaatcattgacatttgggaagactaggaggtgccacagctaaaaggcaccttcgtttcccatcaacgcagctcaagatgactacagataagtttgagacgtaactgagcattctatatcaaatggcactacgcttaagtgtttgggaaacataaatgccacccatcgctggcttttctccatatagcattgtgtctatgtgcgcgggacaaggactgtctagttctgccaaaagaagaaagggaagggtgacctttcccctcactagtaccagcttatatgaaaccaacggaaagaggctacccagtaatcaggccaggggatgaagcactggtttcttgatacacagtttttaaaaggagagctgtgttcatcaactgtgagtgaagtccgtcatgccagagtcccagtgaaaatgcctctatgttataagagactgcaggtccccagacctggctttggctcctctttcttggtcttaggaccttaagccatgtatttgacctctccagctttaatgttggcgttttcaagtggaagtgacactaaattgtgttacctctggttcttaactcccagcattgtgggcaggagcagaagcatcagacatggggtgttaactgtattccagtgtcagtagcatactagatacatggttagcctatgttggccaataacaattcatctttgcaccacctctaagagcctcctgagaagtcgctaatttaaaattaataacagggtgaatatgagctaattagccaaccccttccaggtaagatttccagcggttctgcagtcactcacaagactttgagtcattgctacatttaaagaccatccctcaggagccccgtcagcagcgctgtctccacctagtagccacatcgtggtccctgggagactaatgagattgcaagctcagtcaagtatggccgccaccagccgaaggagcttcaattcctcagaatgtcgtggggaagagaagcagtgattcagttataacaaggagttataatggtcaagtgccagatgacccagactggaggatgtctatgttctcacctatgaagtaaaaggctggcttcagttgacacccactaatctagagagaccttatctgaacccctagtggatgcttgaaacccaaggcagtattgaacccctttgtatttggcttttctgcacatacttatgttaagatttaatttataaattaggcacagtaagagattaactgcagtaactaacagcacaatggcataattagaactataaagctaaaataaaagattcttgaaactaaaaaaaaaaaaaaaaaa

SEQ ID NO: 97 - protein, 95 aa, Rattus norvegicus
MSSPVRTPPPERLEPNPGMSYAVMEIAIIAAVITAVALVLVCLLFLMLRYLYRHKGTYYTNEAKGTEFAESADAALQSDPALQDAGDTSKKEYFI

SEQ ID NO: 98 - DNA, 2743 bp, Rattus norvegicus
  CDS (1)..(2424)
atgtcaggactgagagagtctaagcacatttttaaaagctttgcagggccatactgtgtgtgtgacaacggtaaggacgtagacgctcctggagattataaccagtgcctgcagctctatcggaggaggggactaactgaacgacttgctgactgtgaaggggccatcttggcttctcaagccacggacacaaagagccgggacctgtgtggagtccaggatgctggagctgctgatctgtacaatgatgtgggcaacttgggggttgaggaaatgcaccccaggttagttagtcctgggttctcctatccagtgctgtcagtactagtttatacgctctggttaaatcaagtgtatgactttaaaaggcagacaggttcccttagagagccaccagggctcggccgaggcgtcgcgcctgcgcagtcggccgttcgggcatgcgcgggggctgtggagcgtcgctcggtgaagtctcgcgagagtgagcgccgtggtggaggcacccgcttccccggtgtccctgcgggcccggcgcttggcttcagcttcttccctctgtatctacgcttcccacgcgagttccccttaggcgcacgcccgggctcccgcaggatgggggacaacactagccccatcagcgtgatcctggtgagctcgggcagcaggggcaataagctgctcttcaggtatcccttccagaggagccaggagcaccccgcgtcccagacaactgtgctgatcaggggtttcctagacgtgatacttgacactgtatcagacttactgcgctcagcctactatttccaagatggacgaatgtttgttcctctttataaaccacgtagcagatacgctgtcaacaacacgggagagcatgttgatgaccaggatggtgactccaggttctcggatgctattctggcaacaattttggcaaccaaatctgaaatgtgcggccaaaagtttgagctgaagattgataatgtacggtttgtggggcacccaacactgctgcagcatgctctggggcaggtctccaaaacagatccatctccaaagcgggaagcaccgaccatgatcctttttaatgtggtgtttgcactgagggccaatgctgacccgtcagtgataaactgtctgcacaccttgtcccggcgcattgctaccgtcctgcaacatgaggagcgccgctgccagtacctcacacgggaggccaagctgatcttagcacttcaggatgaggtgtctgctatggccgatgcaaatgaaggtcctcagtccccattccaccacatcctgcccaagtgcaagctggctagggacctcaaagaagcttatgacagcctgtgcacatctggtgtagtacggcttcacattaacagctggctagaagtgagcttctgtcttccccacaagatccactatgcagcttcaagtctgatccctcctgaggctattgaacggagcctgaaagccatccgcccctaccatgctttgctactgctcagtgacgagaagtctctattgagtgagcttcccattgactgctccccggctctggtgcgggtgatcaagaccacgtctgctgtgaagaacctacagcagctagcccaggatgctgatctggccttgctgcaggtcttccagcttgcagctcacctggtgtactggggcaaggccgtcatcatctatccactgtgtgagaacaacgtctatgtcctgtctcccaatgccagtgtgtgtctgtactctccactagctgagcagttctcccgccagttcccgtctcatgacctgccatctgtcctggccaagttttccttgcccgtttctttatcggagttcaggagccccctggccccccctgcacaggagacccagctcatccagatggtggtgtggatgctgcagcgccggctgctcatccagctgcatacctatgtttgcctaatggcatcacccagtgaggaggagccccgacctcgagaggatgacgtccccttcacagcccgagttggtggccgcagcctcagcacacccaatgctctaagctttggctccccaaccagcagtgacgacatgaccctcaccagccccagtatggacaactccagtgcagagctgctccccagtggggactcaccactgaacaaacggatgacagagaacctgctggccagcctctcagagcatgagcgggctgctatcctcaatgtgcccgcagcccaaaaccctgaggacctccgtatgtttgccaggctccttcactacttccgtggccgccaccatctggaggagatcatgtacaatgagaacacacggcgttcccagctgctcatgctctttgacaaattccgcagcgtgctggtggtgaccacccatgaggaccccgttattgctgtcttccaggcactgctcacatgagcccaggtggcatgctgagtgtgtggatggaaacaacctcccctctgctgcagagttcctgactggccttagggggataggcaaccctagcctggcagaactgctccctgtgtcggtcgtcagtctggtcagaagcagctatggggcaaccattgttgggctttgtcttttccctgaaattctagcccagacctcatatttactgttagctaaggatgaccttgaactcctgatcttactgcctgtcctccaagtgctatgactacagatgtgtgccatccatgtcacttttaaaaaatatatttatttattttta

SEQ ID NO: 99 - protein, 808 aa, Rattus norvegicus
MSGLRESKHIFKSFAGPYCVCDNGKDVDAPGDYNQCLQLYRRRGLTERLADCEGAILASQATDTKSRDLCGVQDAGAADLYNDVGNLGVEEMHPRLVSPGFSYPVLSVLVYTLWLNQVYDFKRQTGSLREPPGLGRGVAPAQSAVRACAGAVERRSVKSRESERRGGGTRFPGVPAGPALGFSFFPLYLRFPREFPLGARPGSRRMGDNTSPISVILVSSGSRGNKLLFRYPFQRSQEHPASQTTVLIRGFLDVILDTVSDLLRSAYYFQDGRMFVPLYKPRSRYAVNNTGEHVDDQDGDSRFSDAILATILATKSEMCGQKFELKIDNVRFVGHPTLLQHALGQVSKTDPSPKREAPTMILFNVVFALRANADPSVINCLHTLSRRIATVLQHEERRCQYLTREAKLILALQDEVSAMADANEGPQSPFHHILPKCKLARDLKEAYDSLCTSGVVRLHINSWLEVSFCLPHKIHYAASSLIPPEAIERSLKAIRPYHALLLLSDEKSLLSELPIDCSPALVRVIKTTSAVKNLQQLAQDADLALLQVFQLAAHLVYWGKAVIIYPLCENNVYVLSPNASVCLYSPLAEQFSRQFPSHDLPSVLAKFSLPVSLSEFRSPLAPPAQETQLIQMVVWMLQRRLLIQLHTYVCLMASPSEEEPRPREDDVPFTARVGGRSLSTPNALSFGSPTSSDDMTLTSPSMDNSSAELLPSGDSPLNKRMTENLLASLSEHERAAILNVPAAQNPEDLRMFARLLHYFRGRHHLEEIMYNENTRRSQLLMLFDKFRSVLVVTTHEDPVIAVFQALLT

SEQ ID NO: 100 - DNA, 694 bp, Rattus norvegicus
  misc_feature (660)..(660): n is a, c, g, or t
ttttttttttttttttttgaggttgcctgaatcacggggtttaagagagaagcgttgggttgggggctcacaaccattataagaagggaactaagaaaggtttcaacaagcagataagccaaggtgcaatgtccagtaggaatggcaggccccgcccctttccctgctctctgggagtccaacctggggaaggagctggcaggccccgggtcccctcagttggcagagttacactgtaggatttgctgaggggcaaagagcttcctgacagccgggatctctataggctgccccagcactgagtagttctctttgttgtctgcagtctgggctgaagacttcttaaagtagcacaggcggcaaacagagtggtacttgtccgctccgccaatcacctccacctctttctccaggcccagcctcttggtgtaggaagcttctcgaaagcactccatgcacacagcggtgagcttcaccacactctcggccaagggcaccaggttcaagatgctgccgaaagccttcctctggaaggttccgtccagcgccgccacaatcactgtcttgcctgtattggccatcgtttcacagaaatccacaatgtcagggaaaaactgcccttcatcgatgccaatgacagccacacccaaggcctcctgngccacatccttgagcatgcaggccggcagtgcat

SEQ ID NO: 101 - DNA, 637 bp, Rattus norvegicus
agctgtaaaaacaacatttattactttataatattgagaatgagtatttgggccaaaacaatgggaatgccatacaaataattttttcatcttatatttcaaaacctactacagatattacactgttttttaattgattcacctgtatttgatttgggaaaaatacattgggcaggggacacaaaatatcacacaatagcactaggaaatgtctggaaaataaggggagttgtggattaggatcatgttagctctcatacaggacatatttcagttatgataaaggaagaattgaagcaatcgctggatcccagcaacattccttcagcatctcatctactcctactttctctcaaataatctaagatctctggccttctgttcataaatactccttgactctgtttttgatgagattacttctgaacgcagttttctttctcttcaaaggaccaagacttgagtgtcacactgctgagaattgtcccaggttgtgtccaatatcctcctagcactctccattgtgaatcgtatcaagtagaaaccacctattcccatgtttgctctccacaactcaagtccagaaagtctcaggtacatgcattctgtggcagagaccagctccttgcttgctcaa